(19) United States
(12) Patent Application Publication
(10) Pub. No.: US 2002/0107368 A1
(43) Pub. Date: Aug. 8, 2002

(54) Helicobacter proteins, gene sequences and uses thereof

(76) Inventors: Jing-Hui Tian, Rockville, MD (US); Richard I Walker, Gaithersburg, MD (US); W. James Jackson, Marriottsville, MD (US)

Correspondence Address: PENNIE AND EDMONDS, 1155 AVENUE OF THE AMERICAS, NEW YORK, NY, 100362711

(21) Appl. No.: 09/732,091

(22) Filed: Dec. 6, 2000

(51) Int. Cl. A61K 31/70; C07H 21/04; C07K 1/00; C07K 14/00; C07K 17/00; C07K 16/00; C12P 21/08

(52) U.S. Cl. 530/388.4; 424/190.1; 530/350; 514/44; 536/23.7

(57) ABSTRACT

The invention discloses Helicobacter HP30 or HP56 polypeptide, polypeptides derived thereof (HP30-derived or HP56-derived polypeptides), nucleotide sequences encoding said polypeptides, antibodies that specifically bind the HP30, HP56, HP30-derived or HP56-derived polypeptides and T cells specific for HP30, HP56, HP30-derived or HP56-derived polypeptide. Also disclosed are prophylactic or therapeutic compositions, including immunogenic compositions, e.g. vaccines, comprising HP30, HP56, HP30-derived or HP56-derived polypeptides, nucleic acids encoding the same or antibodies thereto. The invention additionally discloses methods of inducing in animals an immune response to Helicobacter cells.

### 1. FIELD OF INVENTION

The present invention relates to certain Helicobacter species proteins and to the use of these proteins for diagnostic and vaccine applications. In particular the invention relates to polypeptides of the HP56 family and HP30.

The invention further relates to antibodies, including cytotoxic and neutralizing antibodies that are specifically reactive with the proteins of the invention. The invention also relates to T cells specific for the proteins of the invention.

The invention additionally relates to methods of preventing, treating or ameliorating disorders in mammals related to Helicobacter pylori infection and for inducing immune responses to Helicobacter pylori.

The invention further relates to isolated nucleotide sequences and degenerate sequences encoding the proteins of the present invention, vectors having said sequences and host cells containing said vector. Diagnostic methods and kits are also included.

The invention further relates to a method for determining the anti-microbial activity of a substance by evaluating the effect of the substance on the activity of the proteins of the invention.

In other embodiments, the invention, relates to methods for identifying compounds which bind to or otherwise inhibit or activate an activity of a polypeptide or polynucleotide of the invention comprising: contacting a polypeptide or polynucleotides of the invention with a compound to be screened under conditions to permit binding to or other interaction between the compound and the polypeptide or polynucleotide of the invention and determining whether the compound binds to or otherwise interacts with and activates or inhibits the activity of the polypeptide or polynucleotide.

### 2. BACKGROUND OF INVENTION

Helicobacter pylori is a curved, microaerophilic, gram negative bacterium that was isolated for the first time in 1982 from stomach biopsies of patients with chronic gastritis (Warren et al., 1983, Lancet:1273). Originally named Campylobacter pylori, it has been recognized to be part of a separate genus named Helicobacter (Goodwin et al. Int. J. Syst. Bacteriol., 1989, 39:397).

The bacterium colonizes the human gastric mucosa and infection can persist for decades. Infection with H. pylori is one of the most prevalent infections world-wide where approximately 50% of adults in the developed world and over 90% of the inhabitants in the developing world are infected. Chronic infection with H. pylori is believed to be a cause or cofactor of type B gastritis, peptide ulcers, gastric cancers such as adenocarcinoma and low grade B cell lymphoma (see Blaser, 1987, Gastroenterology 93:371; Dooleye et al., 1989, New Eng. J. Med. 321:1562; Personnet et al., 1991, New Engl. J. Med. 325:1127).

H. pylori is believed to be transmitted by the oral route and the risk of infection increases with age (Graham et al., 1991, Gastroenterology 100:1495). In developed countries, the presence of antibodies against H. pylori antigens increases from less than 20% to over 50% in peoples 30 and 60 years old respectively (Jones et al., 1986, Med. Microbiol 22:57). In developing countries over 80% of the population are already infected by the age of 20 (Graham et al., 1991, Digestive Diseases and Sciences 36:1084).

The nature and role of virulence factors of H. pylori are still poorly understood. The factors that have been identified so far include the flagella that are probably necessary to move across the mucus layers, urease that is necessary to neutralize the acidic environment of the stomach and to allow initial colonization and a high molecular weight cytotoxic protein formed by monomers having a molecular weight of 87 Kda that causes formation of vacuoles in eukaryotic epithelial cells and is produced by H. pylori strains associated with disease (Leying et al. Mol. Microbiol., 1992, 6:2863; Cussac et al., 1992, J. Bacteriol. 174:2466; Perez-Perez et al., 1992, J. Infect. Immunol. 60:3658; Cover et al., 1992, J. Biol. Chem. 267:10570).

Numerous therapeutic agents are currently available that eradicate H. pylori infections in vitro (Hopkins et al., 1994, Am. J. Med 97:265). However, many of these agents are suboptimally effective in vivo because of bacterial resistance, altered drug distribution, patients non-compliance or poor drug availability (Hopkins et al., supra). Administration of antibiotics combined with bismuth forms part of the standard regime used to treat H. pylori infection (Malfertheiner et al., 1993, Clinical Therapeutics 15: Supp.B 37-48). Recently combinations of a proton pump inhibitor and single antibiotic have been shown to ameliorate duodenal ulcer disease (Malfertheiner et al. supra). Prevention and treatment of H. pylori infection through immunization is desirable considering the high cost of drug therapy, the appearance of antibiotic resistant strains and the failure of drug therapy to prevent reinfection.

Immunization with H. pylori proteins including urease, heat shock protein, and catalase has resulted in vaccines that induce immune responses to H. pylori but do not protect from colonization upon challenge with H. pylori. (Solnick et al., 2000, Infection and Immunol. 68:2560) Therefore there remains a need to develop vaccines to prevent or treat H pylori infection by inducing immune responses to other antigen(s).

### 3. SUMMARY OF THE INVENTION

One object of this invention is to provide HP56 and HP30 polypeptides from Helicobacter. More particularly, the present invention encompasses HP56 and HP30 polypeptides of Helicobacter pylori, said polypeptides having a molecular weight of about 56 and 30 kDa respectively, with the deduced amino acid sequence of SEQ ID NO:2 (HP56) or SEQ ID NO:4 (HP30), in isolated or recombinant form, as well as fragments of said polypeptides. The present invention encompasses isolated or purified HP30 and HP56 polypeptides, polypeptides derived therefrom (HP30-derived and HP56-derived polypeptides including but not limited to fragments of HP-30 and HP-56), and methods for making said polypeptide and derived polypeptides.

Preferably the HP56 polypeptide has the amino acid sequence depicted in SEQ IN NO:2 or is substantially homologous to SEQ ID NO:2. Preferred fragments of the said polypeptide comprise SEQ ID NOs: 5, 6, 7, 8, 9, 10, 11, 12, 13, 14 or 15.

Preferably the HP30 polypeptide has the amino acid sequence depicted in SEQ IN NO:4 or is substantially homologous to SEQ ID NO:4. Preferred fragments of the said polypeptide comprise SEQ ID NOs:16, 17, 18, 19 or 20.

Another object of the invention is to provide H. pylori fusion peptides having B and/or T cell stimulating activity, preferably comprising at least two T or B cells epitopes derived from the same or from different H. pylori polypeptides which are arranged in a configuration different from a naturally occurring configuration of the regions of the polypeptide.

A preferred polypeptide of the invention is a fusion polypeptide comprising at least two peptides, each of said peptides having an amino acid sequence selected from the group of sequences consisting of the SEQ ID NOS:5, 6,7, 8, 9, 10, 11, 12, 13, 14, 15, 16, 17, 18, 19 and 20, with the proviso that the peptides of the fusion polypeptide are arranged in a configuration that is different from a naturally occurring configuration of HP30 or HP56.

Preferably, the HP30- or HP56-derived polypeptides of the invention are immunologically cross-reactive with the H. pylori peptide protein from which they are derived, and are capable of eliciting in an animal an immune response to H. pylori. A preferred HP30- or HP56-derived polypeptide of the invention induces IgM, IgG, IgA, IgE antibodies, a delayed hypersensitivity T cell response and/or cytotoxic T cell response to cells expressing H. pylori antigen (including but not limited to antigen presenting cells such as macrophages, dendritic cells, B cells, or synthetic antigen presenting cells which display H. pylori antigen), native HP30 or HP56 protein from which the polypeptide is derived, H. pylori cells, or H. pylori cell lysate.

The invention also encompasses antisera and antibodies, including but not limited to neutralizing, cytotoxic or bactericidal polyclonal or monoclonal antibodies, which bind to and are specific for the HP30 or HP56 polypeptide, HP30- or HP56- derived polypeptides and/or fragments thereof.

Preferably the antibodies bind a HP56 or HP30 polypeptide having the amino acid sequence of SEQ ID Nos.:2 or 4. Also included are polyclonal or monoclonal antibodies that specifically bind a HP30- or HP56- derived polypeptide, including but not limited to monoclonal antibodies that specifically bind any of SEQ ID NO:2, 4 or 5-20. Also included are antigen binding fragments of polyclonal or monoclonal antibodies, ie Fv, Fab, Fab′ F(ab′)2 fragments. A further aspect of the invention are chimerized or humanized antibodies in which one or more of the antigen binding regions of the anti- HP30 or HP56 antibody is introduced into the framework region of a heterologous (e.g. human) antibody.

Another aspect of the invention is directed to T cells raised against the antigenic or immunogenic composition(s) of the invention or T cells specific for antigenic or immunogenic polypeptides of the invention or specific for cells expressing H. pylori antigens (including but not limited to antigen presenting cells presenting an HP30 or HP56 polypeptide such as dendritic cells, B cells, or synthetic antigen presenting cells), H. pylon cells, or H. pylori cell lysates.

The invention further provides isolated nucleic acid molecules (DNA or RNA) encoding the HP30 or HP56 polypeptides, HP56-derived polypeptides, HP30-derived polypeptides, vectors having said sequences, host cells containing said vectors, recombinant polypeptides produced therefrom, and pharmaceutical compositions comprising the nucleotide sequences of the nucleic acid molecules, vectors, and cells.

Preferred is the nucleic acid sequence wherein the encoded HP56 or HP30 protein or polypeptide comprises the amino acid sequence of any of SEQ ID Nos.: 2, 4 or 5-20. Also included is an isolated nucleic acid molecule comprising a DNA sequence of any of SEQ ID Nos. 1 or 3 or a complementary sequence thereof, a fragment of the DNA sequence having the nucleic acid sequence of any of SEQ ID Nos.: 1 or 3 or the complimentary sequence thereto; and a nucleic acid molecule which hybridizes under stringent conditions to any one of the sequences described above. The nucleic acid that hybridizes under stringent condition preferably has a sequence homology of about 70%, 80%, 90%, 95%, or 99% with any of the sequences identified above, more preferably about 90%.

The invention further encompasses pharmaceutical compositions including prophylactic or therapeutic compositions, which may be immunogenic compositions including vaccines, comprising one or more of the HP30, HP56, HP30- or HP56-derived polypeptides of the invention, optionally in combination with, fused to or conjugated to one or more other component(s), such other component selected from components including a lipid, phospholipid, a carbohydrate including a lipopolysaccharide, any protein(s) novel or known to those skilled in the art, inactivated whole or attenuated organisms, including but not limited to any virus(es) yeast(s), fungi and bacteria, including but not limited to, Campylobacter spp., Shigella spp., Enteropathogenic E. coli spp, Vibrio cholera or rotavirus.

The invention further encompasses pharmaceutical compositions including prophylactic or therapeutic compositions, which may be immunogenic compositions including vaccines, comprising one or more of the HP30, HP56 polypeptides, HP30-derived or HP56-derived polypeptides and an attenuated or inactivated H. pylori or an attenuated or inactivated H. pylori cultivar expressing HP30 or HP56 polypeptide in a greater amount when compared to wild-type H. pylori.

The invention further encompasses pharmaceutical compositions comprising isolated nucleic acid molecules encoding HP30, HP56 polypeptides, HP30-derived or HP56-derived polypeptides of the present invention which can be used in methods to detect H. pylori infection or to prevent, treat or reduce the severity of a disease or disorder related to infection with H. pylori or H. felis. Such compositions include but are not limited to vectors or recombinant host cells or hosts comprising said nucleic acid molecules.

The invention also includes diagnostic reagents, that may include any one or more of the above mentioned aspects, such as the native HP30 or HP56 proteins, the recombinant HP30 or HP56 proteins, HP30-derived or HP56-derived polypeptides, the nucleic acid molecules, the immunogenic compositions, the antigenic compositions, the antisera, the T cells, the antibodies, the vectors comprising the nucleic acids, and the transformed cells comprising the vectors.

A further aspect of the present invention provides methods for determining the presence of nucleic acids encoding a HP30 or HP56 protein or a HP30-derived or HP56-derived polypeptide in a test sample, and diagnostic kit and reagents therefor, for determining the presence of nucleic acid encoding a HP30 or HP56 polypeptide or HP30-derived or HP56-derived polypeptide.

Also included in this invention are methods of inducing an immune response to Helicobacter spp. and methods of preventing, treating or ameliorating disorders or diseases related to Helicobacter in a mammal, in need of such treatment comprising administering an effective amount of the pharmaceutical or vaccine composition of the invention. Preferred disorders or diseases include a type B gastritis, peptide ulcers, gastric cancers such as adenocarcinoma, and low grade B cell lymphoma. The terms “treatment” or “therapy” as used herein and in the claims encompasses elimination as well as reduction in the severity or amelioration of disease symptoms caused directly or indirectly by the organism or numbers of organisms present.

A further aspect of the invention is antagonists or agonists which inhibit or enhance the activity or expression of the polypeptides or nucleic acid molecules of the invention. Preferred are bacteriostatic or bacteriocidal agonists or antagonists.

A further aspect of the invention is a method for identifying compounds which interact with and inhibit or activate an activity of the polypeptides or nucleic acid molecules of the invention comprising contacting a composition comprising the polypeptide or the nucleic acid molecule with the compound to be screened under conditions to permit interaction between the compound and the polypeptide or nucleic acid molecule to assess the interaction of a compound. The interaction of the compound with the polypeptide or nucleic acid molecule is determined by the association of a second component (e.g. antibody) capable of providing a detectable signal in response to the interaction of the polypeptide or nucleic acid molecule with the compound; and determining the presence or absence of a signal generated from the interaction of the compound with the polypeptide or nucleic acid molecule. Alternatively, the interaction of the compound with the polypeptide or nucleic acid molecule is determined by the ability of the compound to inhibit the activity of the polypeptide or the nucleic acid molecule.

ABBREVIATIONS

| | |
|---|---|
| anti-HP30 | = HP30 polypeptide antibody or antiserum |
| anti-HP56 | = HP56 polypeptide antibody or antiserum |
| ATCC | = American Type Culture Collection |
| immuno-reactive | = capable of provoking a cellular or humoral immune |
| | response |
| kD or kDa | = kilodaltons |
| PBS | = phosphate buffered saline |
| PAGE | = polyacrylamide gel electrophoresis |
| polypeptide | = a peptide of any length, preferably one having eight |
| | or more amino acid residues |
| SDS | = sodium dodecylsulfate |
| SDS-PAGE | = sodium dodecylsulfate polyacrylamide gel |
| | electrophoresis |

Nucleotide or nucleic acid sequences defined herein are represented by one-letter symbols for the bases as follows:

A (adenine)

C (cytosine)

G (guanine)

T (thymine)

U (uracil)

M (A or C)

R(AorG)

W (A or T/U)

S © or G)

Y © or T/U)

K (G or T/U)

V (A or C or G; not T/U)

H (A or C or T/U; not G)

D (A or G or T/U; not C)

B © or G or T/U; not A)

N (A or C or G or T/U) or (unknown)

Peptide and polypeptide sequences defined herein are represented by one-letter symbols for amino acid residues as follows:

A (alanine)

R (arginine)

N (asparagine)

D (aspartic acid)

C (cysteine)

Q (glutamine)

E (glutamic acid)

G (glycine)

H (histidine)

I (isoleucine)

L (leucine)

K (lysine)

M (methionine)

F (phenylalanine)

P (proline)

S (serine)

T (threonine)

W (tryptophan)

Y (tyrosine)

V (valine)

X (unknown)

The present invention may be more fully understood by reference to the following detailed description of the invention, non-limiting examples of specific embodiments of the invention and the appended FIGS.

### 4. BRIEF DESCRIPTION OF DRAWINGS

FIG. 1. Schematic map of the H. pylori HP30 expression plasmid designated “M15(PRE4)PQE/HP30” or more simply “PQE/Hp30” which can be expressed, e.g., in E. coli. In an example, the H. pylori protein is expressed in E. coli as a fusion protein carrying MRGS-(H)₆ GS domain. The sequences of the exemplary expressed recombinant protein and nucleic acid encoding the protein are shown in SEQ ID NOs:44 and 43. The first 12 amino acid residues of the protein expressed by E. coli M15 (Pre4) PQE/HP30 are contributed by vector and comprise the 6×HIS domain, BamHI site and ribosomal binding site. The last nine nucleic acid residues of the schematic map correspond to a stop codon (*) and a Sal I site in the vector.

FIG. 2. Schematic map of the H. pylori HP56 expression plasmid designated “M15(PRE4)PQE/HP56” or more simply “PQE/IP56” which can be expressed, e.g., in E. coli. In an example, the H. pylori protein is expressed in E. coli as a fusion protein carrying MRGS-(H)₆ GS domain. The sequences of the expressed recombinant protein and nucleic acid encoding the protein are shown in SEQ ID NOs:42 and 41. The first 12 amino acid residues of the expressed protein are contributed by vector and comprise the 6×HIS domain, BamHI site and ribosomal binding site. The last nine nucleic acid residues of the schematic map correspond to a stop codon and a Sal I site in the vector.

FIG. 3. A Western blot of gel-purified H. pylori HP30 protein expressed from the E. coli M15(PRE4)PQE/HP30. Lane 1, molecular weight markers (Novex MultiMark); lane 2, non-induced cells; lanes 3 and 4, IPTG induced cells. The HP30 is indicated by an arrow. Molecular weight markers (Lane 1) are Myosin (˜250 kDa), Phosphorylase B (˜148 kDa), GDH (˜60 kDa), CAH (˜42 kDa), Myoglobulin-Blue (˜30 kDa), Myoglobulin-Red (˜22 kDa), Lysozyme(˜17 kDa), Aprotinin (≠6 kDa) and Insulin (˜6) kDa.

FIG. 4. A Coomassie blue stained SDS-Gel of the gel-purified H. pylori HP30 recombinant protein expressed from the M15(PRE4)PQE/HP30 plasmid in E. coli. The protein migrates as a 30 kDa protein. Lane 1, molecular weight markers (Novex MultiMark); lane 2, IPTG induced cells. The HP30 is indicated by an arrow. Molecular weight markers (Lane 1) are Myosin (˜250 kDa), Phosphorylase B (˜148 kDa), GDH (˜60 kDa), CAH (˜42 kDa), Myoglobulin-Blue (˜30 kDa), Myoglobulin-Red (˜22 kDa), Lysozyme(˜17 kDa), Aprotinin (˜6 kDa) and Insulin (˜6) kDa.

FIG. 5. A western Blot of gel purified H. pylori HP56 recombinant protein expressed from the M15(PRE4)PQE/HP56 E.coli. Lanes 1 and 2 IPTG induced cells. Lane 3 molecular weight markers (Novex MultiMark). The HP56 is indicated by an arrow. Molecular weight markers (Lane 1) are Myosin (˜250 kDa), Phosphorylase B (˜148 kDa), GDH (˜60 kDa), CAH (˜42 kDa), Myoglobulin-Blue (˜30 kDa), Myoglobulin-Red (˜22 kDa), Lysozyme(˜17 kDa), Aprotinin (˜6 kDa) and Insulin (˜6) kDa.

FIG. 6. A Coomassie blue stained SDS-gel of E. coli cells carrying the HP56 expression plasmid E. coli M15(PRE4)PQE/HP56. Lane 1, molecular weight markers (Novex MultiMark); lane 2, non-induced cells; lane 3, IPTG induced cells. The HP56 is indicated by an arrow. Molecular weight markers (Lane 1) are Myosin (˜250 kDa), Phosphorylase B (˜148 kDa), GDH (˜60 kDa), CAH (˜42 kDa), Myoglobulin-Blue (˜30 kDa), Myoglobulin-Red (˜22 kDa), Lysozyme(˜17 kDa), Aprotinin (˜6 kDa) and Insulin (˜6) kDa.

FIGS. 7a and 7b. Full length nucleic acid sequence and corresponding amino acid sequence of HP56 polypeptide.

FIG. 8. Full length nucleic acid sequence and corresponding amino acid sequence of HP30 polypeptide.

FIG. 9. Groups of mice were administered a vaccine containing the HP30 recombinant protein alone (50 μg protein/dose) or in combination with several parenteral adjuvants [alum, Freund's complete adjuvant (CFA) or a combination of alum and E.coli heat-labile enterotoxin (LT)]. Three doses of vaccine were given subcutaneously on days 0, 21 and 42. Approximately 14 days after the third dose animals were orally challenged with approximately 5.0×10⁸ cfu H. pylori (Sydney strain) on three consecutive days. Animals were sacrificed approximately 14 days after the third challenge and the stomachs homogenized. The level of H. pylori burden in the stomach was quantified by plating on Brucella Blood agar plates formulated with 6 antibiotics to selectively grow H. pylori. Points on the graph indicate the number of H. pylori cfu measured in the stomach homogenates from individual animals while the bars denote the mean cfu for the group.

FIG. 10. Groups of mice were administered an oral vaccine containing either H. pylori crude cellular lysate or a combination subunit preparation containing the HP30 and HP56 recombinant proteins. The lysate (100 μg protein/dose) and HP30/HP56 antigens (50 μg protein/dose) were administered either alone or with 25 μg of a modified form of E. coli heat-labile enterotoxin (AB5) as an adjuvant. Vaccine was given 3 times on days 0, 14 and 28. Approximately 14 days after the third dose, animals were orally challenged with approximately 5×10⁸ cfu H. pylori (Sydney strain) on three consecutive days. Animals were sacrificed approximately 14 days after the third challenge and stomachs aseptically removed and homogenized. The level of H. pylori burden in the stomach was quantified by plating on Brucella Blood agar plates formulated with 6 antibiotics to selectively grow H. pylori. Points on the graph indicate the number of H. pylori cfu measured in the stomach homogenates from individual animals while the bars denote the mean cfu for the group.

### 5. DETAILED DESCRIPTION OF THE INVENTION

### 5.1. H. pylori Hp30 and Hp56 Polypeptides

The present invention is generally directed to compositions and methods for the diagnosis, prevention, and treatment of Helicobacter infection. In one aspect, the composition of the subject invention provides isolated or pure native (wildtype) or recombinantly produced HP30 and HP56 polypeptides that comprise at least one immunogenic portion of a Helicobacter antigen.

In particular embodiments, the term “Helicobacter” refers to any Helicobacter species (spp.) including but not limited to Helicobacter pylori or Helicobacter felis.

Strains from any of these organism may be obtained worldwide from any biologicals depository, particularly ATCC deposited strains of Helicobacter 43504, 43504D, 43526, 49503, 51652, 51653, 51932, 700392, 700392D 700824D, 51110, 51111, 51407, 51652, 51653, 700392, 700392D, 43504, 43504D, 43526, 43579, 49503, 51110, 51111, 51407, 51211, 51480, 51482, 51630, 51631, 51632, 51800, 51801, 51802, 51863, 51864, 700030, 700031, 700242, 700932, 49286, 49396, 49615, 51101, 51102, 51103, 51104, 51212, 51401, 51402, 51448, 51449, 51450, 51478, 51480, 51482, 51630, 51632, 51800, 51801, 51802, 51863, 51864, 51932, 700030, 700031, 700242, 700824D and 700932.

In a particular embodiment, the Helicobacter protein or polypeptide is a polypeptide comprising a deduced amino acid sequence as depicted in SEQ ID NO:2. In another particular embodiment, the polypeptide is encoded by the nucleotide sequence of SEQ ID NO:1. In another embodiment, the polypeptide comprises an amino acid sequence which is substantially homologous to SEQ ID NO:2 or a portion thereof or is encoded by a nucleotide sequence substantially homologous to the nucleotide sequence having SEQ ID NO:1 or a portion thereof.

In another particular embodiment the Helicobacter protein or polypeptide is a protein comprising a deduced amino acid sequence as depicted in SEQ ID NO:4. In another embodiment, the polypeptide is a polypeptide is encoded by the nucleotide of SEQ ID NO:3. In another embodiment, the Helicobacter polypeptide comprises an amino acid sequence which is substantially homologous to SEQ ID NO:4 or a portion thereof or is encoded by a nucleotide sequence substantially homologous to the nucleotide sequence having SEQ ID NO: 3 or a portion thereof.

As used herein a “substantially homologous” sequence is at least 70%, preferably greater than 80%, more preferably greater than 90% or 95% identical to a reference amino acid or nucleic acid sequence of identical size or when compared to a reference sequence when the alignment or comparison is conducted by a computer homology program or search algorithm known in the art. By way of example and not limitation, useful computer homology programs include the following: Basic Local Alignment Search Tool (BLAST) (www.ncbi.nlm.nih.gov) (Altschul et al., 1990, J. of Molec. Biol., 215:403-410, “The BLAST Algorithm; Altschul et al., 1997, Nuc. Acids Res. 25:3389-3402) a heuristic search algorithm tailored to searching for sequence similarity which ascribes significance using the statistical methods of Karlin and Altschul 1990, Proc. Nat'l Acad. Sci. USA, 87:2264-68; 1993, Proc. Nat'l Acad. Sci. USA 90:5873-77. Five specific BLAST programs perform the following tasks:

1) The BLASTP program compares an amino acid query sequence against a protein sequence database. 2) The BLASTN program compares a nucleotide query sequence against a nucleotide sequence database.

3) The BLASTX program compares the six-frame conceptual translation roducts of a nucleotide query sequence (both strands) against a protein sequence database.

4) The TBLASTN program compares a protein query sequence against a ucleotide sequence database translated in all six reading frames (both strands).

5) The TBLASTX program compares the six-frame translations of a nucleotide query sequence against the six-frame translations of a nucleotide sequence database.

Smith-Waterman (database: European Bioinformatics Institute wwwz.ebi.ac.uk/bic_sw/) (Smith-Waterman, 1981, J. of Molec. Biol., 147:195-197) is a mathematically rigorous algorithm for sequence alignments.

FASTA (see Pearson et al., 1988, Proc. Nat'l Acad. Sci. USA, 85:2444-2448) is a heuristic approximation to the Smith-Waterman algorithm. For a general discussion of the procedure and benefits of the BLAST, Smith-Waterman and FASTA algorithms see Nicholas et al., 1998, “A Tutorial on Searching Sequence Databases and Sequence Scoring Methods” (www.psc.edu) and references cited therein.

By further way of example and not limitation, useful computer homology algorithms and parameters for determining percent identity include the following:

To determine the percent identity of two amino acid sequences or of two nucleic acid sequences, e.g., between HP56 or HP30 sequences and other known sequences, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment, the two sequences are the same length.

The determination of percent identity between two sequences can be accomplished using a mathematical algorithm, A preferred, non-limiting example of a mathematical algorithm utilized for the comparison of two sequences is the algorithm of Karlin and Altschul, 1990, Proc. Nat'l Acad. Sci. USA, 87:2264-68; as modified by 1993, Proc. Nat'l Acad. Sci. USA 90:5873-77. Such algorithm is incorporated into the NBLAST and XBLAST programs of Altschul, 1990, J. of Molec. Biol. 215:403-410. BLAST nucleotide searches can be performed with the NBLAST program, score=100, wordlength =12 to obtain nucleotide sequences homologous to a nucleic acid molecule of the invention. BLAST protein searches can be performed with the XBLAST program, score=50, wordlength=3 to obtain amino acid sequences homologous to a protein molecule of the invention. To obtain gapped alignments for comparison purposes, Gapped BLAST can be utilized as described in Altschul, 1997, Nuc. Acids Res. 25:3389-3402. Alternatively, PSI-BLAST can be used to perform an iterated search which detects distant relationships between molecules (Id.). When utilizing BLAST, Gapped BLAST, and PSI-BLAST programs, the default parameters of the respective programs can be used. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the CGC sequence alignment software package. When using the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, 1994, Comput. Appl. Biosc., 10:3-5; and FASTA described in Pearson and Lipman, 1988, Proc. Nat'l Acad. Sci. USA, 85:2444-2448. Within FASTA, ktup is a control option that sets the sensitivity and speed of the search. If ktup=2, similar regions in the two sequences being compared are found by looking at pairs of aligned residues; if ktup=1, single aligned amino acids are examined. Ktup can be set to 2 or 1 for protein sequences, or from 1 to 6 for nucleotide sequences. The default, if ktup is not specified, is 2 for proteins and 6 for nucleotides. For a further description of FASTA parameters, see, http://bioweb.pasteur.fr/docs/man/man/fasta.1.html#sect2, the contents of which are incorporated herein by reference. Alternatively, protein sequence alignment may be carried out using the CLUSTAL W algorithm as described by Higgins et al., 1996, Methods Enzymol., 266:383-402.

The percent identity between two sequences can be determined using techniques similar to those described above, with or without allowing gaps. In calculating percent identity, only exact matches are counted.

According to various aspects of the invention, the polypeptides of the invention are characterized by their apparent molecular weights based on the polypeptides' migration in SDS-PAGE relative to the migration of known molecular weight markers.

While any molecular weight standards known in the art may be used with the SDS-PAGE, preferred molecular weight markers comprise Phosphorylase B, GDH, CAH, Myoglobulin-Blue, Myoglobulin-Red and Lysozyme.

One skilled in the art will appreciate that the polypeptides of the invention may migrate differently in different types of gel systems (e.g., different buffers; different types and concentrations of gel, crosslinkers or SDS, etc.). One skilled in the art will also appreciate that the polypeptides may have different apparent molecular weights due to different molecular weight markers used with the SDS-PAGE. Hence, the molecular weight characterization of the polypeptides of the invention is intended to be directed to cover the same polypeptides on any SDS-PAGE system and with any set of molecular weight markers which might indicate slightly different apparent molecular weights for the polypeptides than those disclosed herein.

In specific embodiments, the subject invention discloses HP30 or HP56 polypeptides comprising an immunogenic portion of a Helicobacter antigen, wherein the Helicobacter antigen comprises an amino acid sequence encoded by a nucleic acid molecule comprising a sequence selected from the group consisting of (a) nucleotides sequences recited in SEQ ID NO:1 or SEQ ID NO:3, (b) the complements of said nucleotide sequences and (c) variants of such sequences, including but not limited to allelic variants.

### 5.2. Hellicobacter Derived Polypeptides

The term “antigens” and its related term “antigenic” as used herein and in the claims refers to a substance that binds specifically to an antibody or T-cell receptor. As used herein, antisera, antibodies and T cells are “antigen-specific” if they specifically bind to or react with an antigen and do not react detectably with unrelated proteins. Preferably said antigens are immunogenic.

The term “immunogenic” as used herein and in the claims refers to the ability to induce an immune response, e.g., an antibody and/or a cellular immune response in a an animal, preferably a mammal.

In a specific embodiment of the invention, Helicobacter-derived polypeptides consisting of or comprising a fragment of a HP56 protein consisting of at least 8 (continuous) amino acids of the protein are provided. In other embodiments, the fragment consists of at least 10 to 500 amino acids of SEQ ID NO:2. In specific embodiments, such fragments are not larger than 10, 11, 12, 15, 20, 25, 35, 50, 75, 80, 90, 100, 125, 150, 175, 200, 225, 250, 275, 300, 325, 350, 400, 425, or 450 amino acids. In preferred embodiments, the fragments comprise an antigenic or immunogenic epitope of a HP56 polypeptide.

In a particular embodiment, the HP56-derived polypeptide is a fragment of HP56 which comprises any of SEQ ID NOs: 5-15. In another particular embodiment, the HP56-derived polypeptide is a fragment of HP56 which comprises any of SEQ ID NO:5-15 but also comprises additional upstream or downstream HP56 sequences.

In a specific embodiment of the invention, Helicobacter-derived polypeptides consisting of or comprising a fragment of a HP30 protein consisting of at least 8 (continuous) amino acids of the SEQ ID NO:4 are provided. In other embodiments, the fragment consists of at least 10 to 200 amino acids of the SEQ ID NO:4. In specific embodiments, such fragments are not larger than 10, 11, 12, 15, 20, 25, 35, 50, 75, 80, 90, 100, 125, 150, 175, 200, 225, 250 amino acids. In preferred embodiments, the fragments comprise an antigenic or immunogenic epitope of a HP56 polypeptide.

In a particular embodiment, the HP30-derived polypeptide is a fragment of HP30 which comprises any of SEQ ID Nos:16-20. In another particular embodiment, the HP30-derived polypeptide is a fragment of HP30 which comprises any of SEQ ID NO:16-20 but also comprises additional upstream or downstream HP30 sequences.

Preferably, the HP56-derived polypeptides of the invention are immunologically cross-reactive with the HP56 polypeptide, and are capable of eliciting in an animal an immune response to Helicobacter, Helicobacter cell lysates or antigen presenting cells expressing Helicobacter antigen(s).

Preferably the HP30-derived polypeptides of the invention are immunologically cross-reactive with the HP30 polypeptide, and are capable of eliciting in an animal an immune response to Helicobacter, Helicobacter cell lysate(s) or antigen presenting cells expressing Helicobacter antigen(s). More preferably, the HP30-derived or HP56-derived polypeptides of the invention comprise sequences forming one or more epitopes of the native HP56 or HP30 polypeptide of Helicobacter (ie the epitopes of HP56 or HP30 polypeptide as it exists in intact Helicobacter cells). Such preferred HP56-derived or HP30-derived polypeptides can be identified by their ability to elicit an immune response cross-reactive with HP56 or HP30 polypeptide and specifically bind antibodies raised to intact Helicobacter cells (e.g. antibodies elicited by formaldehyde or glutaraldehyde fixed Helicobacter cells or Helicobacter cell lysates; such antibodies are referred to herein as “anti-whole cell” antibodies). For example, HP56 polypeptides or HP30 polypeptide are fractionated using standard methods and tested for their ability to bind anti-whole cell antibodies. Reactive polypeptides are isolated and their amino acid sequence determined by methods known in the art.

Polypeptide derivatives can also be constructed by deletions that remove a part of the parent polypeptide, while retaining the desired specific antigenicity. Deletions can also remove regions of high variability among strains.

Also preferably, the Helicobacter derived polypeptides of the invention comprise sequences that form one or more epitopes of native Helicobacter polypeptide (HP30 or HP56) that mediate bactericidal, neutralizing, or opsonizing antibodies. Such preferred Helicobacter-derived polypeptides may be identified by their ability to generate antibodies that kill Helicobacter spp. particularly, Helicobacter pylori or Helicobacter felis cells. For example, polypeptides from a limited or complete protease digestion or chemical cleavage of HP56 or HP30 polypeptide are fractionated using standard methods, (e.g. by limited proteolytic digestion using enzymes such as trypsin, papain, or related proteolytic enzymes or by chemical cleavage using agents such as cyanogen bromide and followed by fractionation of the digestion or cleavage products), injected into animals and the antibodies produced therefrom tested for the ability to interfere with or kill Helicobacter cells. Once identified and isolated, the amino acid sequences of such preferred Helicobacter-derived polypeptides are determined using standard sequencing methods. The determined sequence may be used to enable production of such polypeptides by synthetic chemical and/or genetic engineering means.

These preferred Helicobacter-derived polypeptides also can be identified by using anti-whole cell antibodies to screen bacterial libraries expressing random fragments of Helicobacter genomic DNA or cloned nucleotide sequences encoding a HP56 or HP-30 polypeptide or fragments thereof. See, e.g., Sambrook et al., Molecular Cloning, A Laboratory Manual, 2nd ed., Cold Spring Harbor Press, NY, Vol. 1, Chapter 12. The reactive clones are identified and their inserts are isolated and sequenced to determine the amino acid sequences of such preferred Helicobacter-derived polypeptides.

Examples of immunogenic portions of antigens contemplated by the present invention include polypeptides comprising or consisting of the fragments set forth in Tables 1 and 2, where the numbers following the HP56 (Table 1, column 1) or HP30 (Table 2, column 1) designation refer to the amino acid residues in SEQ ID NOs 2 or 4, respectively. Polypeptides comprising at least an immunogenic portion of one or more Helicobacter antigens or immunogenic portions as described herein may generally be used, alone or in combination to detect, prevent, treat or reduce the severity of Helicobacter infection.

TABLE 1
HP56 fragments

| HP56 fragment | SEQ ID NO |
|---|---|
| HP56 10-63 | 5 |
| HP56 70-100 | 6 |
| HP56 100-125 | 7 |
| HP56 140-180 | 8 |
| HP56 185-215 | 9 |
| HP56 240-262 | 10 |
| HP56 270-305 | 11 |
| HP5 320-360 | 12 |
| HP56 350-380 | 13 |
| HP56 385-420 | 14 |
| HP56 420-440 | 15 |

TABLE 2
HP30 fragments

| HP30 fragments | SEQ ID NO. |
|---|---|
| HP30 1-30 | 16 |
| HP30 53-90 | 17 |
| HP30 121-150 | 18 |
| HP30 145-185 | 19 |
| HP30 203-251 | 20 |

Polypeptides having a sequence homologous to one of the polypeptides of the invention, include-naturally occurring allelic variants, as well as mutants, variants or any other non-naturally occurring variants that are analogous (i.e., cross-reacting) to a HP56 or HP30 polypeptide of the present invention are encompassed by the present invention.

Allelic variants are very common in nature. For example, a bacterial species e.g. H. pylori, is usually represented by a variety of strains or serovars that differ from each other by minor allelic variations. Indeed, a polypeptide that fulfills the same biological function in different strains can have an amino acid sequence that is not identical in each of the strains. Such an allelic variation may be equally reflected at the nucleic acid molecule level.

An altelic variant is an alternate form of a polypeptide that is characterized as having a substitution, deletion, or addition of one or more amino acids that does not substantially alter the biological function of the polypeptide. By “biological function” is meant the function of the polypeptide in the cells in which it naturally occurs, even if the function is not necessary for the growth or survival of the cells.

Nucleic acid molecules, e.g. DNA molecule, encoding allelic variants can easily be retrieved by the polymerase chain reaction (PCR) amplification of genomic bacterial DNA extracted by conventional methods. This involves the use of synthetic oligonucleotide primers matching upstream and downstream sequences of the 5′ and 3′ ends of the encoding domains. Typically, a primer can consist of 10 to 40, preferably 15 to 25 nucleotides. It may be also advantageous to select primers containing C and G nucleotides in a proportion sufficient to ensure efficient hybridization; e.g. an amount of C and G nucleotides of at least 40%, preferably 50% of the total nucleotide amount.

Variants of H. pylori which share sequence homology or identity to the inventive polypeptide and nucleic acid molecule molecules described herein are also included in the present invention. See Section 5.1. for illustrative methods to determine % homology or identity to a reference sequence of identical size or by alignment or comparison using a computer homology program or search algorithm known in the art. Preferably, the serovar homologues show, 70, 80, 85, 90, 95 or 99% homology or identity to the corresponding polypeptide sequence(s) described herein. Most preferably the serovar homologues show 95-99% homology to the corresponding polypeptide sequence(s) described herein.

A Helicobacter-derived HP56 or HP30 polypeptide includes a fragment or variant thereof i.e., a HP56-derived or HP30-derived polypeptide or fragment having one or more amino acid substitutions, insertions and/or deletions of the wild-type Helicobacter sequence or amino acids chemically modified in vivo or in vitro. Such modifications may enhance the immunogenicity of the resultant Helicobacter -derived polypeptide product or have no effect on such activity. As used herein the term “enhance the immunogenicity” refers to an increased antibody titer or increased cellular immune response as compared to the immune response elicited by unmodified polypeptides or formalin or glutaraldehyde fixed Helicobacter. Modification techniques that may be used include, but are not limited to those disclosed in U.S. Pat. No. 4,526,716.

As an illustrative, non-limiting example, one or more amino acid residues within the HP56- or HP30-derived polypeptide sequence can be substituted by another amino acid of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

Included within the scope of the invention are HP30-derived or HP56-derived polypeptides which are polypeptide fragments or other derivatives or analogs of HP30 or HP56 which are differentially modified during or after translation, e.g., by glycosylation, acetylation, phosphorylation, lipidation, amidation, derivatization by known protecting/blocking groups, proteolytic cleavage, linkage to an antibody molecule or other cellular ligand, etc. Any of numerous chemical modifications may be carried out by known techniques, including but not limited to specific chemical cleavage by cyanogen bromide, trypsin, chymotrypsin, papain, V8 protease, NaBH4; acetylation, formylation, oxidation, reduction; metabolic synthesis in the presence of tunicamycin; etc.

Furthermore, if desired, nonclassical amino acids or chemical amino acid analogs can be introduced as a substitution or addition into the Helicobacter polypeptide sequence. Non-classical amino acids include but are not limited to the D-isomers of the common amino acids, α-amino isobutyric acid, 4-aminobutyric acid, Abu, 2-amino butyric acid, α-Abu, α-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as methyl amino acids, Cα-methyl amino acids, Nα-methyl amino acids, PNA's and amino acid analogs in general. Furthermore, the amino acid can be D (dextrorotary) or L (levorotary).

A HP56 or HP30-derived polypeptide may further be a chimeric polypeptide comprising one or more heterologous polypeptides, lipids, phospholipids or lipopolysaccharides of Helicobacter origin or of another bacterial or viral origin, fused to the amino-terminal or carboxyl-terminal or internal of a complete HP56, or HP30 polypeptide, HP56-derived or HP30-derived polypeptide. Useful heterologous polypeptides comprising such chimeric polypeptides include, but are not limited to, a) pre- and/or pro-sequences that facilitate the transport, translocation and/or processing of the complete HP56, HP30, HP56-derived or HP30-derived polypeptide in a host cell, b) affinity purification sequences, and c) any useful immunogenic sequences (e.g., sequences encoding one or more epitopes of a surface-exposed protein of a microbial pathogen). One preferred heterologous protein of the chimeric polypeptide includes Hin47 (see U.S. Pat. Nos. 5,679,547 and 5,721,115).

HP56- or HP30-derived polypeptides also include but are not limited to fusion polypeptides comprising at least two regions derived from Helicobacter proteins, each having T cell or antibody stimulating activity. The regions may be derived from the same Helicobacter protein or may comprise regions from more than one Helicobacter antigen. The polypeptides are arranged in a nonsequential order or noncontiguous order (e.g. in an order different from the order of the animo acids of the native protein). A preferred polypeptide of the invention is a fusion polypeptide comprising at least two peptides, said peptides consisting of a peptide selected from the group consisting of the SEQ ID NOS:5-20 with the proviso that the peptides of polypeptide are arranged in a configuration that is different from naturally occurring configuration.

Other preferred HP30 or HP56 derived polypeptides of the invention are an isolated fusion polypeptide wherein the polypeptide comprises at least one, preferably at least two, of any of SEQ ID NO 2, 4, 5, 6, 7, 8, 9, 10, 11, 12, 13, 14, 15, 16, 17, 18, 19, or 20 with the proviso that the peptides of said fusion polypeptide are arranged in a configuration that is different from naturally occurring configuration.

If desired, the amino acid sequences of the regions can be produced and joined by a linker. Suitable peptide linker sequences may be chosen based on the following factors: (1) their ability to adopt a flexible extended conformation; (2) their ability to adopt a secondary structure that could interact with functional epitopes of the first and second polypeptides, (3) the lack of hydrophobic or charged residues that might react with the polypeptide functional epitopes; (4) ability to increase solubility and (5) the ability to increase sensitivity to processing by antigen-presenting cells. Such linkers can be any amino acid sequence or other appropriate link or joining agent. Linkers useful in the invention include linkers comprising a charged amino acid pair such as KK or RR, linkers sensitive to cathepsin and or other trypsin-like enzymes, thrombin, Factor Xa or linkers which result in an increase in solubility of the polypeptide. Preferred peptide linkers sequences contain Gly, Asn and Ser residues. Amino acid sequences which may be usefully employed as linkers include those disclosed in Maratea et al., 1985, Gene 40:39-46; Murphy et al., 1986, Proc. Nat. Acad Sci USA 83:8258-8562, U.S. Pat. No. 4,935,233 and U.S. Pat. No. 4,751,180. The linker sequence may be from 1 to about 50 amino acids in length.

Another particular example of fusion polypeptides included in the invention is a polypeptide or polypeptide derivative of the invention fused to a polypeptide having adjuvant activity, such as the subunit B of either cholera toxin or E. coli heat labile toxin. Another particular example of a fusion polypeptide includes a polypeptide or polypeptide derivative of the invention fused to a cytokine (such as, but not limited to, IL-2, IL-10, I1-12, IL-4, interferon). A polypeptide of the invention can be fused to the - or C-terminal end of the polypeptide having adjuvant activity. Alternatively, a polypeptide of the invention can be fused within the amino acid sequence of the polypeptide having adjuvant activity.

Also preferably, the Helicobacter derived fusion polypeptides of the invention comprise sequences that form one or more epitopes of native Helicobacter polypeptide that mediate bactericidal or opsonizing antibodies and/or T cells. Such preferred Helicobacter-derived polypeptides may be identified by their ability to generate antibodies and/or T cells that kill Helicobacter spp or cells expressing HP56 or HP30 epitopes.

### 5.3. Isolation and Purification of Hp56, Hp30, Hp56 Derived or Hp30 Derived Polypeptides

The invention provides isolated HP56, HP30 polypeptides, HP56-derived and HP30-derived polypeptides. As used herein, the term “isolated” means that the product is significantly free of other biological materials with which it is naturally associated. That is, for example, an isolated HP30 polypeptide composition is between about 70% and 99% pure HP30 polypeptide by weight. As used herein, the term “purified” means that the product is substantially free of other biological material with which it is naturally associated. That is, a purified polypeptide composition is at least 70-95% pure polypeptide by weight, preferably at least 98% pure polypeptide by weight, and most preferably at least 99% pure polypeptide by weight.

The HP56 or HP30 polypeptide of the invention may be isolated from a protein extract including a whole cell extract, of any Helicobacter spp., including, but not limited to, Helicobacter pylori or Helicobacter felis. Strains from any of these organisms may be obtained worldwide from any biologicals depository, particularly strains of ATCC 43504D, 43526, 49503, 51652, 51653, 51932, 700392, 700392D 700824D, 51110, 51111, 51407, 51652, 51653, 700392, 700392D, 43504, 43504D, 43526, 43579, 49503, 51110, 51111, 51407, 51211, 51480, 51482, 51630, 51631, 51632, 51800, 51801, 51802, 51863, 51864, 700030, 700031, 700242, 700932, 49286, 49396, 49615, 51101, 51102, 51103, 51104, 51212, 51401, 51402, 51448, 51449, 51450, 51478, 51480, 51482, 51630, 51632, 51800, 51801, 51802, 51863, 51864, 51932, 700030, 700031, 700242, 700824D and 700932.

Another source of the HP56- or HP-30 polypeptide is a protein preparation from a gene expression system expressing a cloned sequence encoding HP56, HP30, HP56-derived polypeptide or HP30-derived polypeptides (see Section 5.5 infra).

The HP56 or HP30 polypeptide can be isolated and purified from the source material using any biochemical technique and approach well known to those skilled in the art. In one approach, Helicobacter cells are lysed and cell debris and removed preferably by centrifugation. The polypeptides in the extract are concentrated, incubated in SDS-containing Laemmli gel sample buffer at 100° C. for 5 minutes and then fractionated by electrophoresis in a denaturing sodium dodecylsulfate (SDS) polyacrylamide gel (PAG) from about 4% to about 12%, with or without a reducing agent. See Laemmli, 1970, Nature 227:680-685. The band or fraction identified as HP30 or HP56 polypeptide, having an apparent molecular weight of 30 kd (HP30) or 56 Kda (HP56), as described above, may then be isolated directly from the fraction or gel slice containing the HP30 or HP56 polypeptide. In a preferred embodiment, HP30 polypeptide has an apparent molecular weight of about 30 kDa which could be determined by comparing its migration distance or rate in a denaturing SDS-PAGE relative to those of Myosin (˜250 kDa), Phosphorylase B (˜148 kDa), GDH (˜60 kDa), CAH (42 kDa), Myoglobulin-Blue (˜30 kDa), Myoglobulin-Red (˜22 kDa) Lysozyme(˜17 kDa), Aprotinin (˜6 kDa) and Insulin (˜6) kDa.

In a preferred embodiment, HP56 polypeptide has an apparent molecular weight of about 56 kDa which could be determined by comparing its migration distance or rate in a denaturing SDS-PAGE relative to those of Myosin (˜250 kDa), Phosphorylase B (˜148 kDa), GDH (˜60 kDa), CAH (˜42 kDa), Myoglobulin-Blue (˜30 kDa), Myoglobulin-Red (˜22 kDa), Lysozyme(˜17 kDa), Aprotinin (˜6 kDa) and Insulin (˜6) kDa.

Another method of purifying HP56 or HP30 polypeptide is by affinity chromatography using anti- HP56 or HP30 antibodies, (see Section 5.4). Polyclonal or monoclonal anti- HP56 or HP30 antibodies are used. Preferred are one or more monoclonal antibodies. The antibodies are covalently linked to agarose gels activated by cyanogen bromide or succinamide esters (Affi-Gel, BioRad, Inc.) or by other methods known to those skilled in the art. The protein extract is loaded on the top of the gel as described above. The contact is for a period of time sufficient to allow the HP56 or HP30 polypeptide to bind to the antibody. Preferably, the solid support is a material used in a chromatographic column. HP56 or HP30 polypeptide is then removed from the antibody, thereby permitting the recovery HP56 or HP30 polypeptide in isolated, or preferably, purified form.

A HP30 or HP56 derived polypeptide of the invention can be produced by chemical and/or enzymatic cleavage or degradation of isolated or purified polypeptide. An HP56 or HP30-derived polypeptide can also be HP56 or HP30 polypeptide fused to a heterologous peptide and the amino acid sequence of the heterologous polypeptide can be produced by methods well known in the art. See, for example, Creighton, 1983, Proteins: Structures and Molecular Principles, W. H. Freeman and Co., NY.

A HP56-derived or HP30-derived polypeptide can also be produced in a gene expression system expressing a recombinant nucleotide construct comprising a sequence encoding HP30 or HP56-derived polypeptide(s). The nucleotide sequences encoding polypeptides of the invention may be synthesized, or cloned, and expressed according to techniques well known to those skilled in the art. See, for example, Sambrook, et al., 1989, Molecular Cloning, A Laboratory, Manual, Vols. 1-3, Cold Spring Harbor Press, NY, Chapter 9.

HP56 derived or HP30-derived polypeptides of the invention can be fractionated and purified by the application of standard protein purification techniques, modified and applied in accordance with the discoveries and teachings described herein.

If desirable, the polypeptides of the invention may be further purified using standard protein or peptide purification techniques including but not limited to electrophoresis, centrifugation, gel filtration, precipitation, dialysis, chromatography (including ion exchange chromatography, affinity chromatography, irnmunoadsorbent affinity chromatography, dye-binding chromatography, size exclusion chromatography, hydroxyappitite chromatography, reverse-phase high performance liquid chromatography, and gel permeation high performance liquid chromatography), isoelectric focusing, and variations and combinations thereof.

One or more of these techniques may be employed sequentially in a procedure designed to isolate and/or purify the HP56, HP30 polypeptide, HP56 derived or the HP30-derived polypeptides of the invention according to its/their physical or chemical characteristics. These characteristics include the hydrophobicity, charge, binding capability, and molecular weight of the protein. The various fractions of materials obtained after each technique are tested for their abilities to bind anti- HP56 or HP30 antibodies or to have functional activity (“test” activities, eg helicase activity). Those fractions showing such activity are then subjected to the next technique in the sequential procedure, and the new fractions are tested again. The process is repeated until only one fraction having the above described “test” activities remains and that fraction produces only a single band or entity when subjected to polyacrylamide gel electrophoresis or chromatography.

### 5.4. Hp56 or Hp30 Immunogens and Antibodies

The present invention provides antibodies that specifically bind HP56, HP30, HP56 derived polypeptides or HP30-derived polypeptides. For the production of such antibodies, isolated or preferably, purified preparations of HP56, HP30, HP56 derived polypeptide or HP30-derived polypeptides are used as immunogens in an immunogenic composition.

In an embodiment, the HP56 or HP30 polypeptide is separated from other proteins present in the extracts of Helicobacter cells using SDS-PAGE (see Section 5.3. above) and the gel slice containing HP56 or HP30 polypeptide is used as an immunogen and injected into an animal (e.g. rabbit) to produce antisera containing polyclonal HP56 or HP30 antibodies. The same immunogens can be used to immunize mice for the production of hybridoma lines that produce monoclonal anti- HP56 or HP30 antibodies. In particular embodiments, the immunogen is a PAGE slice containing isolated or purified HP56 or HP30 from any Helicobacter strain, including, but not limited to, Helicobacter pylori or Helicobacter felis. Particularly preferred are the strains Helicobacter pylori ATCC:43504, 43504D, 43526, 49503, 51652, 51653, 51932, 700392, 700392D 700824D, 51110, 51111, 51407, 51652, 51653, 700392, 700392D, 43504, 43504D, 43526, 43579, 49503, 51110, 51111, 51407, 51211, 51480, 51482, 51630, 51631, 51632, 51800, 51801, 51802, 51863, 51864, 700030, 700031, 700242, 700932, 49286, 49396, 49615, 51101, 51102, 51103, 51104, 51212, 51401, 51402, 51448, 51449, 51450, 51478, 51480, 51482, 51630, 51632, 51800, 51801, 51802, 51863, 51864, 51932, 700030, 700031, 700242, 700824D and 700932.

In other embodiments, peptide fragments of HP56 or HP30 polypeptide are used as immunogens. Preferably, peptide fragments of purified HP56 or HP30 are used. The peptides may be produced by protease digestion, chemical synthesis or recombinantly and then may be isolated or purified. Such isolated or purified peptides can be used directly as immunogens. In particular embodiments, useful peptide fragments are 6 or more amino acids in length. For a discussion of hapten protein conjugates, see, for example, Hartlow, et al., 1988, Antibodies: A Laboratory Manual, Cold Spring Harbor Laboratory Press, Cold Spring Harbor, N.Y., or a standard immunology textbook such as Roitt et al., 1985, IMMUNOLOGY, C.V. Mosby Co., St. Louis, Mo. or Klein, J., 1990, IMMUNOLOGY, Blackwell Scientific Publications, Inc., Cambridge, Mass.

In yet another embodiment, for the production of antibodies that specifically bind one or more epitopes of the native HP56 or HP30 polypeptide, intact Helicobacter or Helicobacter cell lysate are used as immunogen. The cells may be fixed with agents such as formaldehyde or glutaraldehyde before immunization. See Harlow and Lane, 1988, Antibodies:A Laboratory Manual, Cold Spring Harbor Laboratory Press, Cold Spring Harbor, N.Y., Chapter 15. It is preferred that such anti-whole cell antibodies be monoclonal antibodies. Hybridoma lines producing the desired monoclonal antibodies can be identified by using purified HP56 or HP30 polypeptide, intact Helicobacter cells, Helicobacter cell lysates prepared therefrom or cells expressing Helicobacter antigens as the screening ligand. The immunogen for inducing these antibodies are whole cells, extracts or lysates of any Helicobacter, including, but not limited to, Helicobacter pylori or Helicobacter felis. Preferred species are 43504D, 43526, 49503, 51652, 51653, 51932, 700392, 700392D 700824D, 51110, 51111, 51407, 51652, 51653, 700392, 700392D, 43504, 43504D, 43526, 43579, 49503, 51110, 51111, 51407, 51211, 51480, 51482, 51630, 51631, 51632, 51800, 51801, 51802, 51863, 51864, 700030, 700031, 700242, 700932, 49286, 49396, 49615, 51101, 51102, 51103, 51104, 51212, 51401, 51402, 51448, 51449, 51450, 51478, 51480, 51482, 51630, 51632, 51800, 51801, 51802, 51863, 51864, 51932, 700030,700031, 700242, 700824D and 700932.

Polyclonal antibodies produced by Helicobacter cell immunizations contain antibodies that bind other Helicobacter proteins (“non-anti- HP56 or HP30 antibodies”) and thus are more cumbersome to use where it is known or suspected that the sample contains other Helicobacter proteins or materials that are cross-reactive with these other proteins. Under such circumstances, any binding by the anti-whole cell antibodies of a given sample or band must be verified by coincidental binding of the same sample or band by antibodies that specifically bind HP56 or HP30 polypeptide (e.g., anti-HP56, anti-HP30, anti-HP56 derived and/or anti-HP30-derived polypeptide), or by competition tests using anti-HP56, anti-HP30, anti-HP56 derived and/or anti-HP30 as the competitor (i.e., addition of anti-HP56 antibodies, anti-HP30 antibodies, HP56 derived polypeptide, HP30-derived polypeptide to the reaction mix lowers or abolishes sample binding by anti-whole cell antibodies). Alternatively, such polyclonal antisera, containing “non-anti-HP56 or HP30” antibodies, may be cleared of such antibodies by standard approaches and methods. For example, the non-anti-HP30 or HP56 antibodies may be removed by precipitation with cells of Helicobacter strains known not to have the HP56 or HP30 polypeptide; or by absorption to columns comprising such cells or cell lysates of such cells.

In further embodiments, useful immunogens for eliciting antibodies of the invention comprise mixtures of two or more of any of the above-mentioned individual immunogens.

Immunization of animals with the imumunogens described herein, preferably humans, rabbits, rats, ferrets, mice, sheep, goats, cows or horses, is performed following procedures well known to those skilled in the art, for purposes of obtaining antisera containing polyclonal antibodies or hybridoma lines secreting monoclonal antibodies.

Monoclonal antibodies can be prepared by standard techniques, given the teachings contained herein. Such techniques are disclosed, for example, in U.S. Pat. Nos. 4,271,145 and 4,196,265. Briefly, an animal is immunized with the immunogen. Hybridomas are prepared by fusing spleen cells from the immunized animal with myeloma cells. The fusion products are screened for those producing antibodies that bind to the immunogen. The positive hybridomas clones are isolated, and the monoclonal antibodies are recovered from those clones.

Immunization regimens for production of both polyclonal and monoclonal antibodies are well known in the art. The immunogen may be injected by any of a number of routes, including subcutaneous, intravenous, intraperitoneal, intradermal, intramuscular, mucosal, or a combination of these. The immunogen may be injected in soluble form, aggregate form, attached to a physical carrier, as a gel slice, or mixed with an adjuvant, using methods and materials well known in the art. The antisera and antibodies may be purified using column chromatography methods well known to those of skill in the art.

The antibodies may also be used as probes for identifying clones in expression libraries that have inserts encoding HP30 or HP56 polypeptide or fragments thereof. The antibodies, HP56, HP30, HP5 6-derived polypeptides or HP30-derived peptide may also be used in immunoassays (e.g., ELISA, RIA, Westerns) to specifically detect and/or quantitate Helicobacter or anti-Helicobacter antibody in biological specimens. Anti-HP56 or HP30 antibodies of the invention specifically bind HP56 or HP30 polypeptide from Helicobacter pylori or Helicobacter felis. Thus anti-HP30 or HP56 antibodies can be used to diagnose Helicobacter infections.

The antibodies of the invention, including but not limited to those that are cytotoxic, cytostatic, or neutralizing, may also be used in passive immunization to prevent or attenuate Helicobacter infections of animals, including humans. (As used herein, a cytotoxic antibody is one that enhances opsonization and/or complement killing of the bacterium bound by the antibody. As used herein, neutralizing antibody is one that reduces the infectivity of the Helicobacter and/or blocks binding of Helicobacter to target cell). An effective concentration of polyclonal or monoclonal antibodies raised against the immunogens of the invention may be administered to a host to achieve such effects. The exact concentration of the antibodies administered will vary according to each specific antibody preparation, but may be determined using standard techniques well known to those of ordinary skill in the art. Administration of the antibodies may be accomplished using a variety of techniques, including, but not limited to those described in Section 5.7 for the delivery of vaccines.

Another aspect of the invention is directed to antisera raised against the antigenic or immunogenic composition of the invention, and antibodies present in the antisera that specifically bind a HP56 or HP30 protein or a fragment or analogue thereof. Preferably the antibodies bind a polypeptide having the amino acid sequence selected from the group consisting of SEQ ID Nos.: 2 and 4-20 or a HP56 derived or HP30-derived polypeptide. Also included are monoclonal antibodies that specifically bind a polypeptide having the amino acid sequence selected from the group consisting of SEQ ID Nos.: 2 and 4-20.

The term “antibodies” is intended to include all forms, such as but not limited to polyclonal, monoclonal, purified IgG, IgM, or IgA antibodies and fragments thereof, including but not limited to antigen binding fragments such as Fv, single chain Fv (scFv), F(ab.)2, Fab, and F(ab)′ fragments (Harlow et al., 1988, Antibody, Cold Spring Harbor); single chain antibodies (U.S. Pat. No. 4,946,778) and complementary determining regions (CDR), (see Verhoeyen and Windust, 1996, in Molecular Immunology 2ed., by B. D. Hames and D. M. Glover, IRL Press, Oxford University Press, at pp. 283-325), etc.

A further aspect of the invention are chimeric or humanized antibodies (Morrison et al., Proc. Nat'l Acad. Sci. USA 81:6851, 1984; Neuberger et al., Nature 81:6851, 1984) in which one or more of the antigen binding regions of the anti-HP56 or anti-HP30 antibody is introduced into the framework region of a heterologous (e.g., human) antibody. The chimeric or humanized antibodies of the invention are less antigenic in humans than non-human antibodies but have the desired antigen binding and other activities, including but not limited to neutralizing activity, cytotoxic activity, opsonizing activity or protective activity.

A further aspect of the invention is T cells specific for Helicobacter or antigen presenting cells displaying Helicobacter antigens. T cell preparations enriched for T cells specific for HP56, HP30, HP56-derived or HP30-derived polypeptides can be produced or isolated by methods known in the art (See section 5.8).

### 5.5. Nucleic Acids Encoding the Hp30, Hp56, Hp30 Derived or Hp56 Derived Polypeptides

The isolated nucleic acids of the present invention, including DNA and RNA, and comprising a sequence encoding the HP56, HP30, HP56- or HP30-derived polypeptides thereof, may be synthesized using methods known in the art, such as using conventional chemical approaches or polymerase chain reaction (PCR) amplification using convenient pairs of oligonucleotide primers and ligase chain reaction using a battery of contiguous oligonucleotides. The sequences also allow for the identification and cloning of the HP56 or HP30 protein gene from any species of Helicobacter, for instance for screening Helicobacter genomic libraries or expression libraries.

In a particular embodiment, the polypeptide comprises a deduced amino acid sequences as depicted in SEQ ID NOs:2 or 4 and the nucleic acids comprise nucleotide sequences encoding said amino acid sequences. Particularly preferred fragments of HP56 or HP30 have 6 or more deduced amino acid sequences from those depicted in SEQ ID Nos:2 or 4 or sequences substantially homologous thereto and the invention encompasses nucleic acids comprising nucleotides encoding said amino acid sequences. In another particular embodiment, the polypeptide is encoded by the nucleotide sequences of SEQ ID NOs: 1 or 3, with particularly preferred fragments depicted in SEQ ID Nos:21-36 or sequences substantially homologous thereto.

The term “isolated nucleic acid”, “isolated nucleic acid molecule” “isolated nucleotide” or “isolated nucleotide molecule” is defined as a nucleic acid molecule or nucleotide molecule removed from the environment in which it naturally occurs. For example, a naturally-occurring DNA molecule present in the genome of a living bacteria or as part of gene bank is not isolated, but the same molecule separated from the remaining part of the bacterial genome, as a result of e.g. a cloning event (amplification) is isolated. Typically, an isolated DNA molecule is free from DNA regions (e.g., coding regions) with which it is immediately contiguous at the 5′ or 3′ end, in the naturally occurring genome. Such isolated nucleic acid molecules, nucleic acid molecules or nucleotide molecules could be part of a vector or a composition and still be isolated in that such a vector or composition is not part of its natural environment.

Nucleic acids of the present invention can be single or double stranded. The invention also provides nucleic acids hybridizable to or complementary to the SEQ ID NO: 1 or 3 or fragments thereof. In specific aspects, nucleic acids are provided which comprise a sequence fully complementary or complementary to at least 10, 15, 25, 50, 100, 200, 250, 300, 350, 400, 450, 500, 550, 600, 650, 700, 750, 800, 850, 900, 950, 1000, 1100, 1200, 1300 or 1400 contiguous nucleotides of a nucleic acid encoding HP56, HP30, HP56 derived polypeptide or HP30-derived polypeptide. In a specific embodiment, a nucleic acid which is hybridizable to a nucleic acid encoding HP56 or HP30 (e.g., having sequence SEQ ID NO.: 1 or 3), or to a nucleic acid encoding an HP56 derived or HP30-derived polypeptide, under conditions of low, moderate or high stringency is provided.

By way of example and not limitation, procedures using such conditions of low stringency are as follows (see also Shilo and Weinberg, 1981, Proc. Natl. Acad. Sci. USA 78:6789-6792): Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20 ×10⁶ cpm ³²P-labeled probe is used. Filters are incubated in hybridization mixture for 18-20 hour (h) at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and re-exposed to film. Other conditions of low stringency which may be used are well known in the art (e.g., as employed for cross-species hybridizations).

In another specific embodiment, a nucleic acid which is hybridizable to a nucleic acid encoding HP30 or HP56 polypeptide or a HP30 or HP56-derived polypeptide under conditions of high stringency is provided. By way of example and not limitation, rocedures using such conditions of high stringency are as follows: Prehybridization of filters containing DNA is carried out for 8 h to overnight at 65° C. described above with the exception that the annealing temperature is lowered to 50° C. in buffer composed of 6×SSC, 50 mM Tris-HCl (pH 7.5), 1 mM EDTA, 0.02% PVP, 0.02% Ficoll, 0.02% BSA, and 500 mg/ml denatured salmon sperm DNA. Filters are hybridized for 16 or 48 h at 65° C. in prehybridization mixture containing 100 mg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography. Other conditions of high stringency which may be used are well known in the art.

In another specific embodiment, a nucleic acid which is hybridizable to a nucleic acid encoding HP30 or HP56 polypeptide or HP30 or HP56-derived polypeptide under conditions of moderate stringency is provided.

Various other stringency conditions which promote nucleic acid hybridization can be used. For example, hybridization in 6×SSC at about 45 ° C., followed by washing in 2×SSC at 50° C. may be used. Alternatively, the salt concentration in the wash step can range from low stringency of about 5×SSC at 50° C., to moderate stringency of about 2×SSC at 50° C., to high stringency of about 0.2×SSC at 50° C. In addition, the temperature of the wash step can be increased from low stringency conditions at room temperature, to moderately stringent conditions at about 42° C., to high stringency conditions at about 65 ° C. Other conditions include, but are not limited to, hybridizing at 68° C. in 0.5M NaHPO₄ (pH7.2)/ 1 mM EDTA/ 7% SDS, or hybridization in 50% formamide/0.25M NaHPO₄ (pH 7.2)/.25 M NaClIl mM EDTA/7% SDS; followed by washing in 40 mM NaHPO₄ (pH 7.2)/1 mM EDTA/5% SDS at 42° C. or in 40 mM NaHPO₄ (pH7.2) 1 mM EDTA/1% SDS at 50° C. Both temperature and salt may be varied, or alternatively, one or the other variable may remain constant while the other is changed.

Low, moderate and high stringency conditions are well known to those of skill in the art, and will vary predictably depending on the base composition of the particular nucleic acid sequence and on the specific organism from which the nucleic acid sequence is derived. For guidance regarding such conditions see, for example, Sambrook et al., 1989, Molecular Cloning, A Laboratory Manual, Second Edition, Cold Spring Harbor Press, N.Y., pp. 9.47-9.57; and Ausubel et al., 1989, Current Protocols in Molecular Biology, Green Publishing Associates and Wiley Interscience, N.Y.

In the preparation of genomic libraries, DNA fragments are generated some of which will encode parts or the whole of Helicobacter HP30 or HP56 protein. The DNA may be cleaved at specific sites using various restriction enzymes. Alternatively, one may use DNase in the presence of manganese to fragment the DNA, or the DNA can be physically sheared, as for example, by sonication. The DNA fragments can then be separated according to size by standard techniques, including but not limited to, agarose and polyacrylamide gel electrophoresis, column chromatography and sucrose gradient centrifugation. The DNA fragments can then be inserted into suitable vectors, including but not limited to plasmids, cosmids, bacteriophages lambda or T4, bacmids and yeast artificial chromosome (YAC). (See, for example, Sambrook et al., 1989, Molecular Cloning, A Laboratory Manual, 2d Ed., Cold Spring Harbor Laboratory Press, Cold Spring Harbor, New York; Glover, D. M. (ed.), 1985, DNA Cloning: A Practical Approach, MPL Press, Ltd., Oxford, U.K. Vol. 1, II.) The genomic library may be screened by nucleic acid hybridization to labeled probe (Benton and Davis, 1977, Science 196:180; Grunstein and Hogness, 1975, Proc. Natl. Acad. Sci. U.S.A. 72:3961).

The genomic libraries may be screened with labeled degenerate oligonucleotide probes corresponding to the amino acid sequence of any peptide of HP56 or HP30 protein using optimal approaches well known in the art. Any probe used preferably is nucleotides or longer.

The term “probe” as used herein refers to DNA (preferably single stranded) or RNA molecules that hybridize under stringent conditions as defined above, to nucleic acids having sequences identical or homologous to SEQ ID NO:1 or SEQ ID NO:3 or to a complementary or anti-sense sequence. Generally, probes are significantly shorter than full-length sequences shown in SEQ ID NO:1 or 3. For example, they can contain from about 5 to about 100 nucleotides preferably from about 10 to about 80 nucleotides. In particular, probes have sequences that are at least 75% preferably at least 85%, more preferably 95% homologous to a portion of a sequence as shown in SEQ ID NO:1 or 3 or that are complementary to such sequences. Probes can contain modified bases such as inosine, methyl-5-deoxycytidine, deoxyuridine, dimethylamino-5-deoxyuridine, or diamino-2,6 purine.

Clones in libraries with insert DNA encoding the HP56, HP30, HP56-derived or HP30-derived polypeptides will hybridize to one or more of the degenerate oligonucleotide probes. Hybridization of such oligonucleotide probes to genomic libraries is carried out using methods known in the art. For example, hybridization with the two above-mentioned oligonucleotide probes may be carried out in 2×SSC, 1.0% SDS at 50° C. and washed using the same conditions.

In yet another aspect, clones of nucleotide sequences encoding a part or the entire HP56, HP30, HP56-derived or HP30-derived polypeptides may also be obtained by screening Helicobacter expression libraries. For example, Helicobacter DNA or Helicobacter cDNA generated from RNA is isolated and random fragments are prepared and ligated into an expression vector (e.g., a bacteriophage, plasmid, phagemid or cosmid) such that the inserted sequence in the vector is capable of being expressed by the host cell into which the vector is then introduced. Various screening assays can then be used to select for the expressed HP56, HP30, HP56-derived or HP30-derived polypeptides. In one embodiment, the various anti-HP56 or HP30 antibodies of the invention can be used to identify the desired clones using methods known in the art. See, for example, Harlow and Lane, 1988, Antibodies:A Laboratory Manual, Cold Spring Harbor Laboratory Press, Cold Spring Harbor, N.Y., Appendix IV. Clones or plaques from the library are brought into contact with the antibodies to identify those clones that bind.

In an embodiment, colonies or plaques containing DNA that encodes a HP56, HP30, HP56 derived or HP30-derived polypeptides could be detected using DYNA Beads according to Olsvick et al., 1989, 29th ICAAC, Houston, Tex., incorporated herein by reference. Anti-HP56 or HP30 antibodies are crosslinked to DYNA Beads M280, and these antibody-containing beads are used to adsorb to colonies or plaques expressing HP56, HP30, HP56-derived or HP30-derived polypeptides. Colonies or plaques expressing HP56, HP30, HP56-derived or HP30-derived polypeptides are identified as any of those that bind the beads.

Alternatively, the anti-HP56 or HP30 antibodies can be nonspecifically immobilized to a suitable support, such as silica or Celite(tm) resin. This material is used to adsorb to bacterial colonies expressing HP56, HP30, HP56-derived or HP30-derived polypeptides as described in the preceding paragraph.

In another aspect, PCR amplification may be used to produce substantially pure DNA encoding a part of or the whole of HP56 or HP30 protein from Helicobacter genomic DNA. Oligonucleotide primers, degenerate or otherwise, corresponding to known HP56 or HP30 protein sequences can be used as primers.

As examples, an oligonucleotide encoding the N-terminal primer, and together with a 3′ reverse PCR oligonucleotide complementary to an internal, downstream protein coding sequence may be used to amplify an N-terminal-specific HP56 or HP30 DNA fragment. Alternatively, an oligonucleotide encoding an internal HP56 or HP30 coding sequence may be used as the 5′ forward PCR primer together with a 3′ reverse PCR oligonucleotide complementary to downstream, internal HP56 or HP30 protein coding sequences may be used to PCR amplify an internal HP56 or HP30 specific DNA fragment. Alternatively, the forward primer can be combined together with an oligonucleotide complementary to the C-terminal HP56 or HP30 protein coding region to PCR amplify the HP56 or HP30 protein ORF. These HP56 or HP30 protein specific PCR products can be cloned into appropriate expression vectors to direct the synthesis of all or part of the HP56 or HP30 protein polypeptide as distinct proteins or fusion proteins. Alternatively, these HP56 or HP30 protein specific PCR products can be appropriately labeled and used as hybridization probes to identify all or part of the HP56 or HP30 protein gene from genomic DNA libraries.

PCR can be carried out, e.g., by use of a Perkin- Elmer Cetus thermal cycler and Taq polymerase (Gene Amp(tm)). One can choose to synthesize several different degenerate primers, for use in the PCR reactions. It is also possible to vary the stringency of hybridization conditions used in priming the PCR reactions, to allow for greater or lesser degrees of nucleotide sequence similarity between the degenerate primers and the corresponding sequences in Helicobacter DNA. After successful amplification of a segment of the sequence encoding HP56 or HP30 protein protein, that segment may be molecularly cloned and sequenced, and utilized as a probe to isolate a complete genomic clone. This, in turn, will permit the determination of the gene's complete nucleotide sequence, the analysis of its expression, and the production of its protein product for functional analysis, as described infra.

Once a HP56 or HP30 protein polypeptide coding sequence has been isolated from one Helicobacter species, strain, or cultivar, it is possible to use the same approach to isolate HP56 or HP30 protein polypeptide coding sequences from other Helicobacter species, strains and cultivars. It will be recognized by those skilled in the art that the DNA or RNA sequence encoding HP56 or HP30 protein polypeptide (or fragments thereof) of the invention can be used to obtain other DNA or RNA sequences that hybridize with it under conditions of moderate to high stringency, using general techniques known in the art (see supra). Hybridization with HP56 or HP30 protein sequence from one Helicobacter strain or cultivar under high stringency conditions will identify the corresponding sequence from other strains and cultivars. High stringency conditions vary with probe length and base composition. The formulae for determining such conditions are well known in the art. See Sambrook et al., 1989 Molecular Cloning, A Laboratory Manual, Cold Spring Harbor Press, NY, Chapter 11. As an example, high stringency hybridization conditions as applied to probes of greater than 300 bases in length involve a final wash in 0.1×SSC/0.1%SDS at 68° C. for at least 1 hour (Ausbel, et al., Eds., 1989, Current Protocols in Molecular Biology, Vol. 1, Greene Publishing Associates, Inc and John Wiley & Sons, Inc. New York, at page 2.10.2).

One skilled in the art would be able to identify complete clones of HP56 or HP30 protein polypeptide coding sequence using approaches well known in the art. The extent of HP56 or HP30 protein polypeptide coding sequence contained in an isolated clone may be ascertained by sequencing the cloned insert and comparing the deduced size of the polypeptide encoded by the open reading frames (ORFs) with that of HP56 or HP30 protein polypeptide and/or by comparing the deduced amino acid sequence with that of known amino acid sequence of purified HP56 or HP30 protein polypeptide. Where a partial clone of HP56 or HP30 protein polypeptide coding sequence has been isolated, complete clones may be isolated by using the insert of the partial clone as hybridization probe. Alternatively, a complete HP56 or HP30 protein polypeptide coding sequence can be reconstructed from overlapping partial clones by splicing their cloned HP56 or HP30 protein inserts together.

Complete clones may be any that have ORFs with deduced amino acid sequence matching or substantially homologous to that of HP56 or HP30 protein polypeptide or, where the complete amino acid sequence of the latter is not available, that of a peptide fragment of HP56 or HP30 protein polypeptide and having a molecular weight corresponding to that of HP56 or HP30 protein polypeptide. Further, complete clones may be identified by the ability of their inserts, when placed in an expression vector, to produce a polypeptide that binds antibodies specific to the amino-te.nimal of HP56 or HP30 protein polypeptide a nd antibodies specific to the carboxyl-terminal of HP56 or HP3 0 protein polypeptide.

Nucleic acid sequences encoding HP56-derived or HP30-derived polypeptides and fusion proteins thereof may be produced by methods well known in the art. In one aspect, sequences encoding HP56-derived or HP30-derived polypeptides can be derived from HP56 or HP30 polypeptide coding sequences by recombinant DNA methods in view of the teachings disclosed herein. For example, the coding sequence of HP56 or HP30 polypeptide may be altered creating amino acid substitutions that will not affect the immunogenicity of the polypeptide or which may improve its immunogenicity, such as conservative or semi-conservative substitutions as described above. Various methods may be used, including but not limited to oligonucleotide directed, site specific mutagenesis. These and other techniques known in the art may be used to create single or multiple mutations, such as replacements, insertions, deletions, and transpositions, as described in Botstein and Shortle, 1985, Science 229:1193-1210.

Further, DNA of HP56 or HP30 polypeptide coding sequences may be truncated by restriction enzyme or exonuclease digestions. Heterologous coding sequences may be added to HP56 or HP30 polypeptide coding sequence by ligation or PCR amplification. Moreover, DNA encoding the whole or a part of an HP56-derived or HP30 polypeptide may be synthesized chemically or using PCR amplification based on the known or deduced amino acid sequence of the polypeptide and any desired alterations to that sequence.

The identified and isolated DNA containing HP56, HP30, HP56-derived or HP30-derived polypeptide coding sequence can be inserted into an appropriate cloning vector. A large number of vector-host systems known in the art may be used. The term “host” as used herein and in the claims refers to either in vivo in an animal or in vitro in mammalian cell cultures.

Possible vectors include, but are not limited to, plasmids and modified viruses, but the vector system must be compatible with the host cell used. Such vectors include, but are not limited to, bacteriophages such as lambda derivatives, or plasmids such as pET, pBAD, pTrcHis, pBR322 or pUC plasmid derivatives. The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. In an alternative method, the cleaved DNA may be modified by homopolymeric tailing. Recombinant molecules can be introduced into host cells via transformation, transfection, infection, electroporation, etc., so that many copies of the gene sequence are generated.

In an alternative method, the desired DNA containing HP56, HP30, HP56-derived or HP30-derived polypeptide coding sequence may be identified and isolated after insertion into a suitable cloning vector in a “shot gun” approach. Enrichment for the desired sequence, for example, by size fractionation, can be done before insertion into the cloning vector.

In specific embodiments, transformation of host cells with recombinant DNA molecules that contain HP56, HP30, HP56-derived or HP30-derived polypeptide coding sequence enables generation of multiple copies of such coding sequence. Thus, the coding sequence may be obtained in large quantities by growing transformants, isolating the recombinant DNA molecules from the transformants and, when necessary, retrieving the inserted coding sequence from the isolated recombinant DNA.

The nucleotide sequences encoding the polypeptides of the present invention are useful for their ability to selectively form duplex molecules with complementary stretches of other protein genes. Depending on the application, a variety of hybridization conditions may be employed to achieve varying sequence identities. In specific aspects, nucleic acids are provided which comprise a sequence complementary to at least 10, 15, 25, 50, 100, 200 or 250 nucleotides of the HP56 or HP30 protein encoding nucleic acid molecule. In specific embodiments, nucleic acids which hybridize to a HP56 or HP30 protein nucleic acid (e.g. having sequence SEQ ID NO: 1 or 3) under annealing conditions of low, moderate or high stringency conditions.

For a high degree of selectivity, relatively stringent conditions are used to form the duplexes, such as, by way of example and not limitation, low salt and/or high temperature conditions, such as provided by 0.02 M to 0.15 M NaCl at temperatures of between about 50° C. to 70° C. For some applications, less stringent hybridization conditions are required, by way of example and not limitation such a 0.15 M to 0.9 M salt, at temperatures ranging from between about 20° C. to 55° C. Hybridization conditions can also be rendered more stringent by the addition of increasing amounts of formamide, to destabilize the hybrid duplex. Thus, particular hybridization conditions can be readily manipulated, and will generally be a method of choice depending on the desired results.

### 5.6. Recombinatt Production of Hp56, Hp30, Hp56-Derived or Hp30-Derived Polypeptides

In accordance with this invention, it is preferred to make the Helicobacter protein of the present invention by recombinant methods, particularly when the naturally occurring protein as isolated from a culture of a species of Helicobacter may include trace amounts of toxic materials or other contaminants. This problem can be avoided by using recombinantly produced protein of the present invention in heterologous systems which can be isolated from the host in a manner to minimize contaminants in the isolated material. In this case, they are produced by an appropriate host cell that has been transformed by DNA that codes for the polypeptide.

The nucleotide sequence encoding HP30, HP56, HP30 or HP56-derived polypeptides of the invention can be inserted into an appropriate expression vector, i.e., a vector which contains the necessary elements for the transcription and translation of the inserted polypeptide-coding sequence. The nucleotide sequences encoding HP56, HP30 polypeptide, HP56-derived or HP30-derived polypeptides are inserted into the vectors in a manner that they will be expressed under appropriate conditions (e.g., in proper orientation and correct reading frame). The recombinant expression vector also comprises an “expression means”. The term “expression means” refers to elements of a vector which are necessary for transcription and translation of the nucleic acid encoding the protein, including but not limited to promoter/enhancer elements, replication site, an RNA polymerase binding sequence, a ribosomal binding sequence, sequences which are capable of providing phenotype selection (e.g. ampicillin or tetracycline resistance) and replicon and control sequences that can be used to transform host cells. The expression means is operatively coupled to the nucleic acid molecule by linking the inserted nucleic acid molecule into the expression vector.

Promoter/enhancer elements which may be used to control expression of inserted sequences include, but are not limited to the SV40 early promoter region (Bemoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42) for expression in animal cells; the promoters of lactamase (Villa-Kamaroff et al., 1978, Proc. Natl. Acad. Sci. U.S.A. 75:3727-3731), tac (DeBoer et al., 1983, Proc. Natl. Acad. Sci. U.S.A. 80:21-25), or trc for expression in bacterial cells (see also “Useful proteins from recombinant bacteria” in Scientific American, 1980, 242:74-94); the nopaline synthetase promoter region or the cauliflower mosaic virus 35S RNA promoter (Gardner et al., 1981, Nucl. Acids Res. 9:2871), and the promoter of the photosynthetic enzyme ribulose biphosphate carboxylase (Herrera-Estrella et al., 1984, Nature 310:115-120) for expression in plant cells; Gal4 promoter, the ADC (alcohol dehydrogenase) promoter, PGK (phosphoglycerol kinase) promoter, alkaline phosphatase promoter for expression in yeast or other fungi.

Depending on the host-vector system utilized, any one of a number of suitable transcription and translation elements may be used. In a preferred embodiment, a chimeric protein comprising HP56, HP30 protein, HP56-derived or HP30-derived polypeptide sequence and a pre and/or pro sequence of the host cell is expressed. In other preferred embodiments, a chimeric protein comprising HP56, HP30 protein, HP56-derived or HP30-derived polypeptide sequence fused with, for example, an affinity purification peptide, including but not limited to maltose binding protein, glutahione-S-transferase, thioredoxin and histidine tag, is expressed. In further preferred embodiments, a chimeric protein HP56, HP30 protein, HP5 6-derived or HP3 0-derived polypeptide sequence and a useful immunogenic peptide or protein is expressed.

Any method known in the art for inserting DNA fragments into a vector may be used to construct expression vectors containing a HP56, HP30 protein, HP56-derived or HP30-derived polypeptide encoding nucleic acid molecule consisting of appropriate transcriptional/translational control signals and the polypeptide coding sequences. These methods may include in vitro recombinant DNA and synthetic techniques and in vivo recombinants (genetic recombination).

Methods of introducing exogenous DNA into yeast hosts include either the transformation of spheroplasts or of intact yeast cells treated with alkali cations. Transformation procedures usually vary with the yeast species to be transformed. See e.g., Kurtz et al., 1986, Mol. Cell. Biol. 6:142; Kunze et al., 1985, J. Basic Microbiol. 25:141, for Candida; Gleeson et al., 1986, J. Gen. Microbiol. 132:3459; Roggenkamp et al., 1986, Mol. Gen. Genet. 202:302, for Hansenula; Das et al., 1984, J. Bacteriol. 158:1165; De Louvencourt et al.,1983, J. Bacteriol. 154:1165; Van den Berg et al., 1990, Bio/Technology 8:135, for Kluyveromyces; Cregg et al., 1985, Mol. Cell. Biol. 5:3376; Kunze et al., 1985, J. Basic Microbiol. 25:141; U.S. Pat. Nos. 4,837,148 and 4,929,555, for Pichia; Hinnen et al., 1978, Proc. Natl. Acad. Sci. USA 75;1929; Ito et al., 1983, J. Bacteriol. 153:163, for Saccharomyces; Beach et al., 1981, Nature 300:706, for Schizosaccharomyces; Davidow et al., 1985, Curr. Genet. 10:39.

Expression vectors containing HP56, HP30 protein, HP56-derived or HP30-derived polypeptide coding sequences can be identified by three general approaches: (a) nucleic acid hybridization, (b) presence or absence of “marker” gene functions, and (c) expression of inserted sequences. In the first approach, the presence of a foreign gene inserted in an expression vector can be detected by nucleic acid hybridization using probes comprising sequences that are homologous to the inserted HP56, HP30 protein, HP56-derived or HP30-derived polypeptide coding sequence. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of foreign genes in the vector. For example, E. coli may be transformed using pBR322 which contains genes for ampicillin and tetracycline resistance cells. If the HP56, HP30 protein, HP56-derived or HP30-derived polypeptide coding sequence is inserted within the marker gene sequence of the vector, recombinants containing the insert can be identified by the absence of the marker gene fimction. In the third approach, recombinant expression vectors can be identified by assaying the foreign gene product expressed by the recombinant. Such assays can be based, for example, on the physical or functional activity of HP56, HP30 protein, HP56-derived or HP30-derived polypeptide in vitro assay systems, e.g., binding of a His tag to a column, binding to an ligand or receptor, or binding with anti-HP56 or HP30 antibodies of the invention.

Commercially available vectors for expressing heterologous proteins in bacterial hosts include but are not limited to pZERO, pTrc99A, pUC19, pUC18, pKK223-3, pEX1, pCAL, pET, pSPUTK, pTrxFus, pFastBac, pThioHis, pTrcHis, pTrcHis2, and pLEx. For example, the phage in lambda GEM(tm)-1 1 may be utilized in making recombinant phage vectors which can be used to transform host cells, such as E. coli LE392. In a preferred embodiment, the vector is pQE30 or pBAD/ThioE, which can be used transform host cells, such as E. coli.

Expression and transformation vectors for transformation into many yeasts are available. For example, expression vectors have been developed for, the following yeasts: Candida albicans, Kurtz, et al., 1986, Mol. Cell. Biol. 6:142; Candida maltosa, Kunze, et al., 1985, J. Basic Microbiol. 25:141; Hansenula polymorpha, Gleeson, et al., 1986, J. Gen. Microbiol. 132:3459; Roggenkamp et al., 1986, Mol. Gen. Genet. 202:302; Kluyveromyces fragilis, Das, et al., 1984, J. Bacteriol. 158:1165; Kluyveromyces lactis, De Louvencourt et al., 1983, J. BacterioL 154:737; Van den Berg, et al., 1990, Bio/Technology 8:135; Pichia quillerimondii, Kunze et al., 1985, J. Basic Microbiol. 25:141; Pichia pastoris, Cregg, et al., 1985, Mol. Cell. Biol. 5:3376; U.S. Pat. No. 4,837,148 and U.S. Pat. No. 4,929,555; Saccharomyces cerevisiae, Hinnen et al., 1978, Proc. Natl. Acad. Sci. USA 75:1929; Ito et al., 1983, J. Bacteriol. 153:163; Schizosaccharomyces pombe, Beach et al., 1981, Nature 300:706; and Yarrowia lipolytica, Davidow, et al., 1985, Curr. Genet. 10:380471 Gaillardin, et al., 1985, Curr. Genet. 10:49.

A variety of host-vector systems may be utilized to express the polypeptide-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage DNA, plasmid DNA, or cosmid DNA, plant cells or transgenic plants. Hosts that are appropriate for expression of nucleic acid molecules of the present invention, fragments, analogues or variants thereof, may include E. coli, Bacillus species, Haemophilus, fungi, yeast, such as Saccharomyces, Pichia, Bordetella, or Candida or the baculovirus expression system may be used. Preferably, the host cell is a yeast or bacterium. Particularly desirable hosts for expression in this regard include Gram positive bacteria which do not have LPS and are, therefore endotoxin free. Most preferably the bacterium is E. coli, B. subtilis or Salmonella.

In addition, a host cell strain may be chosen which modulates the expression of the inserted sequences, or modifies and processes the gene product in the specific fashion desired. Expression from certain promoters can be elevated in the presence of certain inducers; thus, expression of the genetically engineered HP30, HP56, HP30-derived or HP56-derived polypeptide may be controlled. Furthermore, different host cells have characteristic and specific mechanisms for the translational and post-translational processing and modification of proteins. Appropriate cell lines or host systems can be chosen to ensure the desired modification and processing of the foreign protein expressed.

Once a suitable host system and growth conditions are established, recombinant expression vectors can be propagated and prepared in quantity. Upon expression, a recombinant polypeptide of the invention is produced and can be recovered in a substantially purified from the cell paste, the cell extract or from the supernatant after centrifugation of the recombinant cell culture using techniques well known in the art. For instance, the recombinant polypeptide can be purified by antibody-based affinity purification, preparative gel electrophoresis, or affinity purification using tags (e.g. 6× histidine tag) included in the recombinant polypeptide. (See section 5.3).

### 5.7. Compositions

The present invention also provides therapeutic and prophylactic compositions, which may be antigenic compositions, and preferably immunogenic compositions including vaccines, against Helicobacter infections of animals, including mammals, and more specifically rodents and primates, including humans. Preferred immunogenic compositions include vaccines for use in humans. The antigenic, preferably immunogenic, compositions of the present invention can be prepared by techniques known to those skilled in the art and comprise, for example, an immunologically effective amount of any of the HP30 or HP56 immunogens disclosed in Sections 5.1. or 5.2 optionally in combination with or fused to or conjugated to one or more other immunogens, including a lipid, phospholipid, carbohydrate, lipopolysaccharide, inactivated or attenuated whole organism(s) and other protein(s), of Helicobacter origin or other bacterial origin, a pharmaceutically acceptable carrier, optionally an appropriate adjuvant, and optionally other materials traditionally found in vaccines. Such a cocktail vaccine (comprising several immunogens) has the advantage that immunity against one or several strains of a single pathogen or one or several pathogens can be obtained by a single administration. Examples of other immunogens include, but are not limited to, those used in the known DPT vaccines, H. pylori cytotoxin (Covacci et al. 2000 U.S. Pat. No. 6,130,059), H. pylori heat shock protein (hsp60) (Covacci et al. 2000 US 6,077,706), H. pylori CagA (Covacci et al. 2000 U.S. Pat. No. 5,928,865), H. pylori urease (Michetti et al. 1999 US 5,972,236), H. pylon catalase (Doidge et al. 1999 U.S. Pat. No. 6,005,000), H. pylori nickel binding protein (Plaut et al. 1999 U.S. Pat. No. 5,972,348, H. pylori tagA (Cover et al. 1999 U.S. Pat. No. 5,876,943), H. pylori enolase (Thompson et al. 1997 U.S. Pat. No. 5,703,219), entire attenuated or killed organisms or subunits therefrom of Campylobacter spp., Shigella spp., Enteropathogenic E. coli spp, Vibrio cholera or rotavirus.

The term “immunologically effective amount” is used herein to mean an amount sufficient to induce an immune response to produce antibodies, T cells, and/or cytokines and other cellular immune response components. Preferably, the immunogenic composition is one that prevents Helicobacter infections or attenuates the severity of any preexisting or subsequent Helicobacter infection. An immunologically effective amount of the immunogen to be used in the vaccine is determined by means known in the art in view of the teachings herein. The exact concentration will depend upon the specific immunogen to be administered, but can be determined by using standard techniques well known to those skilled in the art for assaying the development of an immune response.

The composition elicits an immune response in a subject. Compositions which induce antibodies, including anti-HP56 or anti-HP30 protein antibodies and antibodies that are neutralizing, opsonizing or bactericidal are one aspect of the invention. According to preferred, non-limiting, embodiments of the invention, an effective amount of a composition of the invention produces an elevation of antibody titer to at least three times the antibody titer prior to administration. In a preferred, specific, non-limiting embodiment of the invention, approximately 0.01 to 2000 μg and preferably 0.1 to 500 μg are administered to a host. Compositions which induce T cells responses which are bactericidal or reactive with cells (e.g., antigen presenting cells, including but not limited to, dendritic cells and macrophages) expressing Helicobacter antigen(s) are also an aspect of the invention. Preferred are compositions additionally comprising an adjuvant. Preferred are compositions additionally comprising an antibiotic which has bactericidal activity against H. pylori, including but not limited to, meprazole, clarithromycin, omeprazole, metronidazole, tetracycline, Lansoprazole or amoxicillin.

The combined immunogen and carrier or diluent may be an aqueous solution, emulsion or suspension or may be a dried preparation. In general, the quantity of polypeptide immunogen will be between 0.1 and 500 micrograms per dose. The carriers are known to those skilled in the art and include stabilizers, diluents, and buffers. Suitable stabilizers include carbohydrates, such as sorbitol, lactose, mannitol, starch, sucrose, dextran, and glucose and proteins, such as albumin or casein. Suitable diluents include saline, Hanks Balanced Salts, and Ringers solution. Suitable buffers include an alkali metal phosphate, an alkali metal carbonate, or an alkaline earth metal carbonate.

The immunogenic compositions, including vaccines, of the invention are prepared by techniques known to those skilled in the art, given the teachings contained herein. Generally, an immunogen is mixed with the carrier to form a solution, suspension, or emulsion. One or more of the additives discussed above may be in the carrier or may be added subsequently. The vaccine preparations may be desiccated, for example, by freeze drying or spray drying for storage or formulations purposes. They may be subsequently reconstituted into liquid vaccines by the addition of an appropriate liquid carrier or administered in dry formulation known to those skilled in the art, particularly in capsules or tablet forms.

An effective amount of the antigenic, immunogenic, pharmaceutical, including, but not limited to vaccine, composition of the invention should be administered, in which “effective amount” is defined as an amount that is sufficient to produce a desired prophylactic, therapeutic or ameliorative response in a subject, including but not limited to an immune response. The amount needed will vary depending upon the immunogenicity of the HP56, HP30 protein, HP56-derived or HP30-derived polypeptide, nucleic acid used, and the species and weight of the subject to be administered, but may be ascertained using standard techniques.

Immunogenic, antigenic, pharmaceutical and vaccine compositions may further contain one or more auxiliary substance, such as wetting or emulsifying agents, pH buffering agents, or adjuvants to enhance the effectiveness thereof. Immunogenic, antigenic, pharmaceutical and vaccine compositions may be administered to humans or other mammals including ruminants, rodents or primates, parenterally, intradermally, intraperitoneal, subcutaneously or intramuscularly.

Alternatively, the immunogenic, antigenic, pharmaceutical and vaccine compositions formed according to the present invention, may be formulated and delivered in a manner to evoke an immune response at mucosal surface(s). Thus, the immunogenic, antigenic, pharmaceutical and vaccine compositions may be administered to mucosal surface(s) by, for example, the nasal, oral, ocular, bronchiolar, intravaginal or intrarectal routes. Alternatively, other modes of administration including suppositories and oral formulations may be desirable. For suppositories, binders and carriers may include, for example, polyalkalene glycols or triglycerides. Oral formulations may include normally employed incipients such as, for example, pharmaceutical grades of saccharine, cellulose and magnesium carbonate. These compositions can take the form of microspheres, nanospheres, solutions, suspensions, tablets, pills, capsules, sustained release formulations or powders and contain about 0.001 to 95% of the HP56, HP30 protein, HP56-derived or HP30-derived protein. The immunogenic, antigenic, pharmaceutical and vaccine compositions are administered in a manner compatible with the dosage formulation, and in such amount as will be therapeutically or prophylactically effective, protective or immunogenic. Preferred are compositions additionally comprising an adjuvant.

Further, the immunogenic, antigenic, pharmaceutical and vaccine compositions may be used in combination with or conjugated to one or more targeting molecules for delivery to specific cells of the immune system, such as the mucosal surface. Some examples include but are not limited to vitamin B12, bacterial toxins or fragments thereof, monoclonal antibodies and other specific targeting lipids, proteins, nucleic acids or carbohydrates.

The quantity to be administered depends on the subject to be treated, including, for example, the capacity of the individual's immune system to synthesize antibodies, and if needed, to produce a cell-mediated immune response. Precise amounts of active ingredient required to be administered depend on the judgment of the practitioner. However, suitable dosage ranges are readily determinable by one skilled in the art and may be of the order of 0.1 to 1000 micrograms of the HP56, HP30 protein, HP56-derived or HP30-derived polypeptide. Suitable regimes for initial administration and booster doses are also variable, but may include an initial administration followed by subsequent administrations. The dose may also depend on the route(s) of administration and will vary according to the size of the host. The concentration of the HP56, HP30 protein, HP56-derived or HP30-derived polypeptide in an antigenic, immunogenic or pharmaceutical composition according to the invention is in general about 0.001 to 95%.

The antigenic, immunogenic or pharmaceutical preparations, including vaccines, may comprise as the immunostimulating material a nucleotide vector comprising at least a portion of the nucleic acid molecule encoding the HP56, HP30 protein, HP56-derived or HP30-derived polypeptide.

A vaccine can comprise nucleic acid molecule molecules encoding one or more HP56, HP30 protein, HP56-derived or HP30-derived polypeptides or fusion proteins as described herein, such that the polypeptide is generated in situ. In such vaccines, the nucleic acid molecules may be present within any of a variety of delivery systems known to those of ordinary skill in the art, including nucleic acid expression systems, bacterial and viral expression systems. Appropriate nucleic acid expression systems contain the necessary nucleic acid molecule sequences for expression in the patient such as suitable promoter and terminating signals. In a preferred embodiment, the nucleic acid molecules may be introduced using a viral expression system (e.g. vaccinia or other pox virus, alphavirus retrovirus or adenovirus) which may involve the use of non-pathogenic (defective) virus. Techniques for incorporating nucleic acid molecules into such expression systems are well known to those of ordinary skill in the art. The nucleic acid molecules may also be administered as “naked” plasmid vectors as described, for example in Ulmer et al., 1992, Science 259:1745-1749, and reviewed by Cohen, 1993, Science 259:1691-1692. Techniques for incorporating DNA into such vectors are well known to those of ordinary skill in the art. A retroviral vector may additionally transfer or incorporate a gene for a selectable marker (to aid in the identification or selection of transduced cells) and/or a targeting moiety, such as a gene that encodes a ligand for a receptor on a specific target cell, to render the vector target specific. Targeting may also be accomplished using an antibody, by methods known to those of ordinary skill in the art.

Nucleic acid molecules (DNA or RNA) of the invention can be administered as vaccines for therapeutic or prophylactic purpose. Typically a DNA molecule is placed under the control of a promoter suitable for expression in a mammalian cell. The promoter can function ubiquitously or tissue-specifically. Examples of non-tissue specific promoters include the early cytomegalovirus (CMV) promoter (described in U.S. Pat. No. 4,168,062) and Rous Sarcoma virus promoter (described in Norton and Coffin, 1985, Molec. Cell Biol. 5:281. The desmin promoter (Li et al., 1989, Gene 78:243, Li Paulin,1991, J. Biol Chem 266:6562 and Li & Paulin, 1993, J. Biol Chem 268:10401) is tissue specific and drives expression in muscle cells. More generally, useful vectors are described in i.a., W094/21797 and Hartikka et al., 1996, Human Gene Therapy 7:1205.

A composition of the invention can contain one or several nucleic acid molecules of the invention. It can also contain at least one additional nucleic acid molecule encoding another Helicobacter antigen or fragment derivative including but not limited to H. pylori cytotoxin (Covacci et al. 2000 U.S. Pat. No. 6,130,059), H. pylori heat shock protein (hsp60) (Covacci et al. 2000 U.S. Pat. No. 6,077,706), H. pylori CagA (Covacci et al. 2000 U.S. Pat. No. 5,928,865), H. pylori urease (Michetti et al. 1999 U.S. Pat. No. 5,972,236), H. pylori catalase (Doidge et al. 1999 U.S. Pat. No. 6,005,000), H. pylori nickel biding protein (Plaut et al. 1999 U.S. Pat. No. 5,972,348, H. pylori tagA (Cover et al. 1999 U.S. Pat. No. 5,876,943) and H. pylori enolase (Thompson et al. 1997 U.S. Pat. No. 5,703,219). A nucleic acid molecule encoding a cytokine, such as interleukin-1, inteleukin-4 interleukin-12 or interferon can also be added to the composition so that the immune response is enhanced. DNA molecules of the invention and/or additional DNA molecules may be on different plasmids or vectors in the same composition or can be carried in the same plasmid or vector.

Other formulations of nucleic acid molecules for therapeutic purposes included sterile saline or sterile buffered saline, colloidal dispersion systems, such as macromolecule complexes, nanocapsules, silica microparticles, tungsten microparticles, gold microparticles, microspheres, beads and lipid based systems including oil-in-water emulsions, micelles, mixed micelles and liposomes. A preferred colloidal system for use as a delivery vehicle in vitro and in vivo is a liposome(ie an artifical vesicle). The uptake of naked nucleic acid molecules may be increased by incorporating the nucleic acid molecules into and/or onto biodegradable beads, which are efficiently transported into the cells. The preparation and use of such systems is well known in the art.

A nucleic acid molecule can be associated with agent(s) that assist in cellular uptake. It can be formulated with a chemical agent that modifies the cellular permeability, such as bupivacaine (see e.g. WO94/16737).

Cationic lipids are also known in the art and are commonly used for DNA delivery. Such lipids include LipofectinTM also knows as DOTMA (N-[1-(2,3-dioleyloxy)propyl]-N,N,N-trimethylammonium chloride), DOTAP (1,2-bis(oleyloxy)-3-(trimethylammonio)propane, DDAB (dimethyldioctadecylammonium bromide), DOGS (dioctadecylamidologlycy spermine) and cholesterol derivatives such as DC-Chol (3 beta-(N-(N′,N′-dimethyl aminomethane)-carbamoyl) cholesterol. A description of these cationic lipids can be found in EP 187,702, WO 90/11092, U.S. Pat. No. 5,283,185, WO 91/15501, WO 95/26356, and U.S. Pat. No. 5,527,928. Cationic lipids for DNA delivery are preferably used in association with a neutral lipid such as DOPE (dioleyl phosphatidylethanolamine) as described in e.g. WO 90/11092.

Other transfection facilitation compound(s) can be added to a formulation containing cationic liposomes. They include i.a., spermine derivatives useful for facilitating the transport of DNA through the nuclear membrane (see, for example, WO 93/18759) and membrane-permeabilizing compounds such as GALA, Gramicidine S and cationic bile salts (see, for example, WO 93/19768).

The amount of nucleic acid molecule to be used in a vaccine recipient depends, e.g. on the strength of the promoter used in the DNA construct, the immunogenicity of the expressed gene product, the mode of administration and type of formulation. In general, a therapeutically or prophylactically effective dose from about 1 mg to about 1 mg, preferably from about 10 mg to about 800 mg and more preferably from about 25 mg to about 250 mg can be administered to human adults. The administration can be achieved in a single dose or repeated at intervals.

The route of administration can be any conventional route used in the vaccine field. As general guidance, a nucleic acid molecule of the invention can be administered via a mucosal surface, e.g. an ocular, intranasal, pulmonary, oral, intestional, rectal, vaginal, and urinary tract surface; or via a parenteral route, e.g., by an intravenous, subcutaneous, intraperitoneal, intradermal, intra-epidermal or intramuscular route. The choice of administration will depend on the formulation that is selected. For instance a nucleic acid molecule formulated in association with bupivacaine is advantageously administered into muscles.

Recombinant bacterial vaccines genetically engineered for recombinant expression of nucleic acid molecules encoding HP56, HP30 protein, HP56-derived or HP30-derived polypeptides including Shigella, Salmonella, Vibrio cholerae, Lactobacillus, BCG and Streptococcus can also be used for prevention or treatment of Helicobacter infections. Non-toxicogenic Vibrio cholerae mutant strains that are useful as a live oral vaccine are described in Mekalanos et al, Nature 306:551 1983 and U.S. Pat. No. 4,882,278. An effective vaccine dose of a Vibrio cholerae strain capable of expressing a polypeptide or polypeptide derivative encoded by a DNA molecule of the invention can be administered. Preferred routes of administration include all mucosal routes, most preferably intranasally or orally.

Attenuated Salmonella typhimurium strains, genetically engineered for recombinant expression of heterologous antigens or not and their use as oral vaccines are described in Nakayama et al., 1988, Bio/Technology 6:693 and WO 92/11361. Preferred routes of administration include all mucosal routes, most preferably intranasally or orally.

Other bacterial strains useful as vaccine vectors are described in High et al., 1992, EMBO 11:1991; Sizemore et al., 1995, Science 270:299 (Shigella flexneri); Medaglini et al., 1995, Proc Natl. Acad. Sci. US 92:6868 (Streptococcus gordonii); and Flynn, 1994, Cell Mol. Biol.40:31, WO 88/6626, WO 90/0594, WO 91/13157, WO 92/1796 and WO 02/21376 (Bacille Calmette Guerin).

In genetically engineered recombinant bacterial vectors, nucleic acid molecule(s) of the invention can be inserted into the bacterial genome, carried on a plasmid, or can remain in a free state.

When used as vaccine agents, recombinant bacterial vaccines, nucleic acid molecules and polypeptides of the invention can be used sequentially or concomitantly as part of a multistep immunization process. For example, a mammal can be initially primed with a vaccine vector of the invention such as pox virus, e.g. via the parenteral route and then boosted several time with the a polypeptide e.g. via the mucosal route. In another example, a mammal can be vaccinated with polypeptide via the mucosal route and at the same time or shortly thereafter, with a nucleic acid molecule via intramuscular route.

An adjuvant can also be added to a vaccine composition containing a recombinant bacteria. To efficiently induce humoral immune responses (HIR) and cell-mediated immunity (CMI), immunogens are typically emulsified in adjuvants. Immunogenicity can be significantly improved if the immunogen is co-administered with an adjuvant. Adjuvants may act by retaining the immunogen locally near the site of administration to produce a depot effect facilitating a slow, sustained release of antigen to cells of the immune system. Adjuvants can also attract cells of the immune system to an immunogen depot and stimulate such cells to elicit immune responses.

Many adjuvants are toxic, inducing granulomas, acute and chronic inflammations (Freund's complete adjuvant, FCA), cytolysis (saponins and Pluronic polymers) and pyrogenicity, arthritis and anterior uveitis (LPS and MDP). Although FCA is an excellent adjuvant and widely used in research, it is not licensed for use in human or veterinary vaccines because of its toxicity.

Immunostimulatory agents or adjuvants have been used for many years to improve the host immune responses to, for example, vaccines. Intrinsic adjuvants, such as lipopolysaccharides, normally are the components of the killed or attenuated bacteria used as vaccines. Extrinsic adjuvants are immunomodulators which are typically non-covalently linked to antigens and are formulated to enhance the host immune responses. Thus, adjuvants have been identified that enhance the immune response to antigens delivered parenterally. Aluminum hydroxide, aluminum oxide, and aluminum phosphate (collectively commonly referred to as alum) are routinely used as adjuvants in human and veterinary vaccines. The efficacy of alum in increasing antibody responses to diphtheria and tetanus toxoids is well established and a HBsAg vaccine has been adjuvanted with alum.

Other extrinsic adjuvants may include chemokines, cytokines, (e.g. IL-2) saponins complexed to membrane protein antigens (immune stimulating complexes), pluronic polymers with mineral oil, killed mycobacteria in mineral oil, Freund's complete adjuvant, bacterial products, such as muramyl dipeptide (MDP) and lipopolysaccharide (LPS), as well as lipid A, and liposomes.

International Patent Application, PCT/US95/09005 incorporated herein by reference describes mutated forms of heat labile toxin of enterotoxigenic E. coli (“mLT”). U.S. Pat. No. 5,057,540, incorporated herein by reference, describes the adjuvant, QS21, an HPLC purified non-toxic fraction of a saponin from the bark of the South American tree Quiliaja saponaria molina 3D-MPL is described in great Britain Patent 2,220,211, and is incorporated herein by reference.

U.S. Pat. No. 4,855,283 granted to Lockhoff et al on Aug. 8, 1989 which is incorporated herein by reference, teaches glycolipid analogues including N-glycosylamides, N-glycosylureas and N-glycosylcarbamates, each of which is substituted in the sugar residue by an amino acid, as immuno-modulators or adjuvants. Lockhoff reported that N-glycosphospholipids and glycoglycerolipids, are capable of eliciting strong immune responses in both herpes simplex virus vaccine and pseudorabies virus vaccine. Some glycolipids have been synthesized from long chain-alkylamines and fatty acids that are linked directly with the sugars through the anomeric carbon atom, to mimic the functions of the naturally occurring lipid residues.

U.S. Pat. No. 4,258,029 granted to Moloney, incorporated herein by reference thereto, teaches that octadecyl tyrosine hydrochloride (OTH) functioned as an adjuvant when complexed with tetanus toxoid and formalin inactivated type I, II and III poliomyelitis virus vaccine. Lipidation of synthetic peptides has also been used to increase their immunogenicity.

Therefore, according to the invention, the immunogenic, antigenic, pharmaceutical, including vaccine, compositions comprising a HP56, HP30, HP56-derived or HP30-derived polypeptide or a nucleic acid encoding a polypeptide of the invention or fragment thereof, vector or cell expressing the same, may further comprise an adjuvant, such as, but not limited to alum, mLT, (modified labile toxin of enteropathogenic E. coli) QS21, MMPL, CpG DNA, MF59, calcium phospgate, PLG and all those listed above. Preferably, the adjuvant is selected from one or more of the following: alum, QS21, CpG DNA, PLG, LT, 3D-mPL, or Bacille Calmette-Guerine (BCG) and mutated or modified forms of the above, particularly mLT, e.g., LTR192G or AB5. The compositions of the present invention may also further comprise a suitable pharmaceutical carrier, including but not limited to saline, bicarbonate, dextrose or other aqueous solution. Other suitable pharmaceutical carriers are described in Remington's Pharmaceutical Sciences, Mack Publishing Company, a standard reference text in this field, which is incorporated herein by reference in its entirety.

Immunogenic, antigenic and pharmaceutical, including vaccine, compositions may be administered in a suitable, nontoxic pharmaceutical carrier, may be comprised in microcapsules, and/or may be comprised in a sustained release implant.

Immunogenic, antigenic and pharmaceutical, including vaccine, compositions may desirably be administered at several intervals in order to sustain antibody levels and/or T cell levels. Immunogenic, antigenic and pharmaceutical, including vaccine, compositions may be used in conjunction with other bacteriocidal or bacteriostatic methods.

Another embodiment of the vaccines of the present is a vaccine comprising one or more:

a) an isolated HP56 of Helicobacter spp, having a molecular weight of 56 kDa as determined in SDS polyacrylamide gel electrophoresis;

b) an isolated HP30 of Helicobacter spp, having a molecular weight of 30 kDa as determined in SDS polyacrylamide gel electrophoresis;

c) an isolated nucleic acid encoding an isolated HP56 polypeptide of Helicobacter spp, having a molecular weight of 56 kDa as determined in SDS polyacrylamide gel electrophoresis; or

d) an isolated nucleic acid encoding an isolated HP30 polypeptide of Helicobacter spp, having a molecular weight of 30 kDa as determined in SDS polyacrylamide gel electrophoresis and further comprising one or more components selected of from the group consisting of alum, mLT, QS21, MF59, CpG DNA, MPL, calcium phosphate and PLG.

Also included in the invention is a method of producing an immune response in an animal comprising immunizing the animal with an effective amount of one or more of the polypeptides of the invention or nucleic acid molecules encoding one of polypeptides of the invention, compositions comprising same and vaccines comprising same. The polypeptides of the invention, nucleic acids, compositions and vaccines comprising same of the invention may be administered simultaneously or sequentially. Examples of simultaneous administration include where two or more polypeptides, nucleic acids, compositions, or vaccines, which may be the same or different, are administered in the same or different formulation or are administered separately, e.g. in a different or the same formulation but within a short time (such as minutes or hours) of each other. Examples of sequential administration include where two or more polypeptides, nucleic acids, compositions or vaccines which may be the same or different are not administered together within a short time of each other, but may be administered separately at intervals of for example days, weeks, months or years.

Also included in the invention is treating or ameliorating a disease associated with Helicobacter infection by administering an antibiotic with Helicobacter bactercidal activity prior to, simultaneously, or sequentially with any of the vaccine compositions of the invention.

The polypeptide, nucleic acid molecule or recombinant bacterial vaccines of the present invention are also useful in the generation of antibodies as described supra or T cells. For T cells, animals, including humans, are immunized as described above. Following immunization, peripheral blood cells (PBL), spleen cells or lymph node cells are harvested and stimulated in vitro by placing large numbers of lymphocytes in flasks with media containing serum. A polypeptide of the present invention is added. T cells are harvested and placed in new flasks with X-irradiated peripheral blood mononuclear cells. The polypeptide is added directly. Cells are grown in the presence of IL-2. As soon as the cells are shown to be Helicobacter specific T cells, they are changed to a stimulation cycle with higher IL-2 (20 units) to expand them.

Alternatively, one or more T cells that proliferate in the presence of a polypeptide of the present invention can be expanded in number by cloning.

Methods for cloning cells are well known in the art. For example, T cell lines may be established in vitro and cloned by limiting dilution. Responder T cells are purified from the peripheral blood established in culture by stimulating with the nominal antigen in the presence of irradiated autologous filler cells. In order to generate CD4+ T cell lines, the Helicobacter polypeptide is used as the antigenic stimulus and autologous PBL or lymphoblastoid cell lines (LCL) immortalized by infection with Epstein Barr virus are used as antigen presenting cells. In order to generate CD8+ T cell lines, autologous antigen-presenting cells transfected with an expression vector which produces relevant Helicobacter polypeptide may be used as stimulator cells. T cell lines are established following antigen stimulation by plating stimulated T cells in 96-well flat-bottom plates with PBL or LCL cells and recombinant interleukin-2 (rIL2) (50 U/ml). Wells with established clonal growth are identified at approximately 2-3 weeks after initial plating and restimulated with appropriate antigen in the presence of autologous antigen-presenting cells, then subsequently expanded by the addition of low doses of rIL2. T cell clones are maintained in 24-well plates by periodic restimulation with antigen and rIL2 approximately every two weeks.

T cell preparations may be further enriched by isolating T cells specific for antigen reactivity using the methods disclosed by Kendricks et al. in U.S. Pat. No. 5,595,881.

The vaccine compositions of the present inventions are useful in preventing, treating or ameliorating disease symptoms in an animal with a disease or disorder associated with Helicobacter infection. Such diseases or disorders include, but are not limited to, Helicobacter bacterial infection, type B gastritis, peptide ulcers, gastric cancers such as adenocarcinoma and low grade B cell lymphoma.

### 5.8. Immunoassays and Diagnostic Reagents

The HP56 or HP30 polypeptides or nucleic acid encoding same, and fragments thereof are useful as a diagnostic reagent. An antigen or immunogen for the generation of anti-HP56 or anti-HP30 antibodies or as an antigen in immunoassays including enzyme-linked immunosorbent assays (ELISA), radioimmmnunoassays (RIA) and other non-enzyme linked antibody binding assays or procedures known in the art for the detection of anti-bacterial, anti-Helicobacter, and anti-HP56 or HP30 protein antibodies are encompassed by the invention.

In ELISA assays, the protein is immobilized onto a selected surface, for example, a surface capable of binding proteins such as the wells of a polystyrene microtiter plate. After washing to remove incompletely absorbed protein, a nonspecific protein solution that is known to be antigenically neutral with regard to the test sample may be bound to the selected surface. This allows for blocking of nonspecific absorption sites on the immobilizing surface and thus reduces the background caused by nonspecific bindings of antisera onto the surface.

The immobilizing surface is then contacted with a sample, such as clinical or iological materials, to be tested in a manner conducive to immune complex (antigen/antibody) formation. This may include diluting the sample with diluents, such as solutions of bovine gamma globulin (BGG) and/or phosphate buffered saline (PBS)/Tween. The sample is then allowed to incubate for from 2 to 4 hours, at temperatures such as of the order of about 20° C. to 37° C. Following incubation, the sample-contacted surface is washed to remove non-immunocomplexed material. The washing procedure may include washing with a solution, such as PBS/Tween or a borate buffer. Following formation of specific immunocomplexes between the test sample and the bound protein, and subsequent washing, the occurrence, and even amount, of immunocomplex formation may be determined by subjecting the immunocomplex to a second antibody having specificity for the first antibody. If the test sample is of human origin, the second antibody is an antibody having specificity for human immunoglobulins and in general IgG.

To provide detecting means, the second antibody may have an associated activity such as an enzymatic activity that will generate, for example, a color development upon incubating with an appropriate chromogenic substrate. Detection may then be achieved by detecting color generation. Quantification may then be achieved by measuring the degree of color generation using, for example, a visible spectrophotometer and comparing to an appropriate standard. Any other detecting means known to those skilled in the art are included.

In Western blot assays, the polypeptide either as a purified preparation or a cell extract, is submitted to SDS-PAGE electrophoresis as described by Laemmli, 1970, Nature 227:690. After transfer to a nitrocellulose membrane, the material is further incubated with the serum sample, polyclonal antibody preparation, or monoclonal antibody diluted in the range of dilutions from about 1:5 to 1:5000, preferably from about 1:100 to about 1:500. The reaction is revealed according to standard procedures. For example, when human antibody is used, the membrane is incubated in a goat anti-human peroxidase conjugate for an appropriate length of time. The membrane is washed. The reaction is developed with the appropriate substrate and stopped. The reaction is measured visually by the appearance of a colored band e.g. by colorimetry.

In a dot blot assay, the purified or partially purified polypeptide or cell extract can be used. Briefly, a solution of the antigen at about 100μg/ml is serially two-fold diluted in 50 mM Tris-HCL (pH7.5). 100 ml of each dilution are applied to a nitrocellulose membrane 0.45 um set in a 96-well dot blot apparatus. The buffer is removed by applying vacuum to the system. Wells are washed by addition of 50 μM Tris-HCl (pH 7.5) and the membrane is air-dried. The membrane is saturated in block buffer (50 mM Tris-HCl (pH 7.5), 0.15 M NaCl and 10 g/L skim milk) and incubated with an antiserum dilution from about 1:50 to about 1:500. The reaction is revealed according to standard procedures. For example, a goat anti-rabbit peroxidase conjugate is added to the well when rabbit antibodies are used. Incubation is carried out 90 minutes at 37° C. and the blot is washed. The reaction is developed with the appropriate substrate and stopped. The reaction is measured visually by the appearance of a colored spot, e.g. by colorimetry.

The HP56, HP30, HP56-derived or HP30- derived polypeptide or nucleic acid encoding same, and fragments thereof are also useful as an antigen or immunogen for the generation of anti-HP56 or HP30 protein T cell responses or as an antigen in immunoassays including T cell proliferation assays, cytokine production, delayed hypersensitivity reactions or cytotoxic T cells (CTL) reactions.

For analysis of Helicobacter peptide specific T cell proliferative responses, fresh peripheral blood, spleen or lymph node cells are harvested. Cells are plated into 96-well round bottom microtiter plates and are incubated with peptides. Data is expressed as a stimulation index (SI) which is defined as the mean of the experimental wells divided by the mean of the control wells (no antigen). Analysis of the phenotype (e.g. CD4+ or CD8+) of Helicobacter specific T cells can be determined by, immunofluorence staining, FACS analysis or by depletion with appropriate antisera.

For analysis of cytokine release of T cells in response to Helicobacter polypeptides, responder cells are mixed with polypeptides. Supernatants are collected and added to an enzyme-linked immunosorbent assay (ELISA) coated with antibody to the cytokine (e.g. anti-IFN-γ or anti-IL-2 antibody). After washing, rabbit anti-cytokine polyclonal antibody (e.g. anti-IFN-γ or anti-IL-2) is added. Labeled goat anti-rabbit IgG polyclonal is added. Substrate is added and the amount of cytokine released into the supernatant is determined based upon the amount of color developed in the ELISA test.

Another embodiment includes diagnostic kits comprising all of the essential reagents required to perform a desired immunoassay according to the present invention. The diagnostic kit may be presented in a commercially packaged form as a combination of one or more containers holding the necessary reagents. Such a kit comprises HP56, HP30, HP56-derived or HP30-polypeptide or nucleic acid encoding same or a monoclonal or polyclonal antibody of the present invention in combination with several conventional kit components. Conventional kit components will be readily apparent to those skilled in the art and are disclosed in numerous publications, including Antibodies A Laboratory Manual (E. Harlow, D. Lane, 1989, Cold Spring Harbor Laboratory Press) which is incorporated herein by reference in its entirety. Conventional kit components may include such items as, for example, microtiter plates, buffers to maintain the pH of the assay mixture (such as, but not limited to Tris, HEPES, etc.), conjugated second antibodies, such as peroxidase conjugated anti-mouse IgG (or any anti-IgG to the animal from which the first antibody was erived) and the like, and other standard reagents as well as instructions for performing a desired assay or test.

The nucleic acid sequences of the present invention may be used in combination with an appropriate indicator means, such as a label, for determining hybridization. A wide variety of appropriate indicator means are known in the art, including radioactive, enzymatic or other ligands, such as avidin/biotin and digoxigenin-labeling, which are capable of providing a detectable signal. In some diagnostic embodiments, an enzyme tag such as urease, alkaline phosphatase or peroxidase, instead of a radioactive tag may be used. In the case of enzyme tags, colorimetric indicator substrates are known which can be employed to provide a means visible to the human eye or spectrophotometrically, to identify specific hybridization with samples containing HP56 or HP30 protein gene sequences.

Probes of the invention can be used in diagnostic tests, as capture or detection probes. Such capture probes can be conventionally immobilized on a solid support directly or indirectly, by covalent means or by passive adsorption. A detection probe can be labeled by a detection marker selected from radioactive isotopes, enzymes such as peroxidase, alkaline phosphatase, and enzymes able to hydrolyze a chromogenic, fluorogenic or luminescent substrate; compounds that are chromogenic fluorogenic or luminescent; nucleotide base analogs; and biotin.

Probes of the invention can be used in any conventional hybridization techniques such as dot blot (Maniatis et al., 1982, Molecular Cloning: A Laboratory Manual Cold Spring Harbor Laboratory Press, Cold Spring Harbor, N.Y.), Southern blot (Southern, 1975, J. Mol. Biol. 98:503, northern blot (identical to Southern blot to the exception that RNA is used as a target), or sandwich techniques (Dunn et al., 1977, Cell 12:23).

In embodiments involving solid-phase procedures, the test DNA (or RNA) from samples, such as clinical samples, including exudates, body fluids (e.g., serum, amniotic fluid, middle ear effusion, sputum, semen, urine, tears, mucus, bronchoalveolar lavage fluid) or even tissues, is absorbed or otherwise affixed to a selected matrix or surface. The fixed, single-stranded nucleic acid is then subjected to specific hybridization with selected probes comprising the nucleic acid sequences of the protein encoding genes or fragments or analogues thereof of the present invention under desired conditions. The selected conditions will depend on the particular circumstances based on the particular criteria required depending on, for example, the G+C contents, type of target nucleic acid, source of nucleic acid, size of hybridization probe etc. Following washing of the hybridization surface so as to remove non-specifically bound probe molecules, specific hybridization is detected, or even quantified, by means of the label. It is preferred to select nucleic acid sequence portions that are conserved among species of Helicobacter. The selected probe may be at least 15 bp and may be in the range of about 30 to 90 bp.

### 5.9. Applications

The proteins, polypeptides, peptides, antibodies, T cells and nucleic acids of the invention are useful as reagents for clinical or medical diagnosis of Helicobacter infections and for scientific research on the properties of pathogenicity, virulence, and infectivity of Helicobacter, as well as host defense mechanisms. For example, DNA and RNA of the invention can be used as probes to identify the presence of Helicobacter in biological specimens by hybridization or PCR amplification. The DNA and RNA can also be used to identify other bacteria that might encode a polypeptide related to the Helicobacter HP56 or HP30 protein. The proteins of the invention may be used to prepare polyclonal and monoclonal antibodies that can be used to further purify compositions containing the proteins of the invention by affinity chromatography or for use as diagnostic or for use as prophylactic or therapeutic agents. The proteins can also be used in standard immunoassays to screen for the presence of antibodies or T cells to Helicobacter in a biological sample.

### 5.10. Biological Deposits

Certain plasmids that contain portions of the gene having the open reading frame of the HP30 and HP56 genes encoding the Helicobacter proteins of the present invention have been inserted into E. coli and deposited with the American Type Culture Collection (ATCC) located at 10801 University Boulevard, Manassas, Va. 20110-2209, U.S.A., pursuant to the Budapest Treaty and pursuant to 37 CFR 1.808 and prior to the filing of this application. The identifications of the respective portions of the genes present in these plasmids are shown below.

Samples of the deposited materials will become available to the public upon grant of a patent based upon this United Stated patent application. The invention described and claimed herein is not to be limited by the scope of the plasmids deposited, since the deposited embodiment is intended only as an illustration of the invention. Any equivalent or similar plasmids that encode similar or equivalent proteins or fragments or analogues thereof as described in this application are within the scope of the invention.

| Biological Deposit | ATCC Accession No. | Date Deposited |
|---|---|---|
| E. coli M15(PRE4)PQE/HP30 | ATCC PTA-2670 | Nov. 15, 2000 |
| E. coli M15(PRE4)PQE/HP56 | ATCC PTA-2669 | Nov. 15, 2000 |

### 6. Examples

The above disclosure generally describes the present invention. A more complete understanding can be obtained by reference to the following examples. The examples are described solely for the purpose of illustration and are not intended to limit the scope of the invention. Changes in form and substitution of equivalents are contemplated as circumstances suggest or render expedient. Although specific terms have been employed herein, such terms are intended in a descriptive sense and not for purposes of limitation.

Methods of molecular genetics, protein biochemistry and immunology used but not explicitly described in the disclosure and examples are amply reported in the scientific literature and are well within the ability of those skilled in the art.

### 6.1. Growth of H. pylon

H pylori G1-4 (hereafter referred to as G1-4) was isolated from a patient with duodenal ulcer. Stock cultures of G1-4 were stored at −70° C. in brain heart infusion broth (Difco Laboratories, Sparks, Md.) supplemented with 15% glycerol and 4% heat-inactivated bovine calf serum. H. pylori was cultured in brain heart infusion (BHI) medium supplemented with 4% heat-inactivated fetal calf serum for 24-48 hours in a microaerobic atmosphere. Two milliliters of thawed stock culture were transferred to a 500 ml shake flask containing 50 ml of BHI supplemented with heat-inactivated fetal calf serum or bovine calf serum. The culture was flushed with a mixed gas (5% O₂, 10% CO₂ and 85% N₂) and incubated at 37° C. with 150 rpm agitation.

### 6.2. Amino Terminal Sequencing of Hp30 and Hp56 Polypeptide

To obtain the N-terminal amino acid sequence, sufficient quantities of the HP30 or HP56 protein (>5 mg) are electroblotted onto a PVDF membrane (Applied Biosystems), and stained with Coomassie blue. Immobilized protein is released from the membrane and treated in situ with low levels of endopeptidase Lys-C, endopeptidase Arg-C and/or endopeptidase Glu-C to fragment the native protein. The resulting peptide fragments are purified by HPLC and their N-terminal amino acid sequences are determined using an ABI 430 Protein Sequenator and standard protein sequencing methodologies.

### 6.3. Isolation of GHelicobacter pylori Chromosomal DNA

Helicobacter pylori strain G1-4 was streaked for single colonies on Campylobacter Chocolate agar plates containing TVAP (Remel) and grown overnight at 37° C. under microaerobic atmosphere. Three or four single colonies were picked and used to inoculate a ˜1.5 ml broth seed culture (BHI broth containing 4% bovine calf serum) which was grown overnight in a shaking incubator, ˜150 rpm, at 37° C. A 500 ml Erlemneyer flask containing ˜50 ml of BHI broth was inoculated with the seed culture and grown for ˜24-48 hours at 37° C. under microaerobic atmosphere in a shaking incubator, ˜175 rpm, to generate cell mass for DNA isolation. Cells were collected by centrifugation in a Sorvall GSA rotor at ˜2000×g for 15 minutes at room temperature. The supernatant was removed and the cell pellet suspended in ˜5.0 ml of sterile water. An equal volume of lysis buffer (200 mM NaCl, 20 mM EDTA, 40 mM Tris-HCl pH8.0, 0.5% (w/v) SDS, 0.5% (v/v) 2-mercaptoethanol, and 250 μg/ml of proteinase K) was added and the cells suspended by gentle agitation and trituration. The cell suspension was then incubated ˜12 hours at 50° C. to lyse the bacteria and liberate chromosomal DNA. Proteinaceous material was precipitated by the addition of 5.0 ml of saturated NaCl (˜6.0M, in sterile water) and centrifugation at ˜5,500×g in a Sorvall SS34 rotor at room temperature. Chromosomal DNA was precipitated from the cleared supernatant by the addition of two volumes of 100% ethanol. Aggregated DNA was collected and washed using gentle agitation in a small volume of a 70% ethanol solution. Purified chromosomal DNA was suspended in sterile water and allowed to dissolve/disburse overnight at 4° C. by gentle rocking. The concentration of dissolved DNA was determined spectrophoto-metrically at 260 nm using an extinction coefficient of 1.0 O.D. unit ˜50 mg/ml.

### 6.4. Identification of an Open Reading Frame in H. pylori with Homology to LeIf of Leishmania

The Leishmania major initiation factor 4A (LeIF) of Leishmania has been shown to be an adjuvant enhancing T cell immune responses (see WO 99/29341). To determine if an homologous protein is produced in H. pylori, the LeIF amino acid sequence available from GeneBank was employed as a BLAST (TBLASTN) subject query to search the Helicobacter pylori genomic sequence database (The Institute for Genomic Research, Rockville, Md.) to potentially identify linear amino acid sequences that might share some similarity with the LeIF protein. No predicted amino acid sequences from this H. pylori database showed more than ˜50-55% similarity to the LeIF protein sequence. Candidate amino acid sequences from the Helicobacter pylori database were derived computationally within specific genomic DNA sequence “contigs” and putative open reading frames encoding short relevant sequences. Putative ORFs believed to be capable of encoding proteins of ˜50 Kdal, the size of the L. major LeIF, were then selected. Several putative open reading frames were identified from the H. pylori genome which met these criteria. One putative H. pylori ORF encoding a protein meeting most of the searching criteria was designated HP56 and chosen for subsequent cloning, expression, and analysis as an adjuvant.

### 6.5. PCR Amplification of Hp56 Orf-specific DNA Fragements

The polymerase chain reaction (PCR) was employed to generate HP56 specific DNA fragments for expression cloning and genetic variability analysis. An N-terminal PCR forward primer was chemically synthesized that encodes the DNA sequence for the first ˜7 amino acids of the protein (i.e. the ˜21 nucleotide sequence beginning with the Met translation initiation). In addition to the ORF- specific sequence, the forward PCR primer also contained a short 5′ G/C clamp (˜6 nucleotides) for efficient PCR amplification. A BamHI restriction endonuclease cleavage site for use in subcloning was appropriately engineered into this primer between the G/C clamp and the ORF-specific sequence.

The sequence of the HP56 N-terminal PCR forward strand primer is: HP56-Bam-F 5′-CAG AGG GGA TCCATG GAA TTG AAT CAA CCA CCA-3′ (SEQ ID NO:37)

The ORF-specific sequence is in bold and the BamiHI restriction site is underlined.

An oligonucleotide having a DNA sequence complementary to that encoding the last ˜7 amino acids of the HP56 ORF protein, beginning with the endogenous stop codon (TAA) was synthesized and employed as a reverse PCR primer. Like the forward PCR primer, the reverse primer contained a short G/C clamp (˜6 nucleotide) for efficient DNA amplification and a SalI restriction endonuclease site appropriately positioned for subcloning.

The sequence of the HP56 C-terminal PCR reverse strand primer is: HP56-Sal-RC 5′-CAG AGG GTC GACTTA ACG GCG TTT GGG TTT TTT AGA-3′ (SEQ ID NO:38)

The ORF-specific sequence is in bold and the SalI restriction site is underlined.

Oligonucleotides were synthesized on an Applied Biosystems Inc. (ABI) Model 380B DNA synthesizer using a 0.2 nmol scale column (ending mode: trityl-on, auto-cleavage) and standard phosphoramidite chemistry. Crude oligonucleotides were manually purified over C18 reverse phase syringe columns (OPC columns, ABI) as described by the manufacturer. Purity and yield were ascertained spectrophotometrically (230/260/280 ratios). Standard PCR amplification reactions (2 mM Mg²⁺, 200 μmol dNTPs, 2.5 units recombinant AmpliTaq (PE Biosystems), in a 200 μl final reaction volume) were programmed using about 0.5 μg H. pylori G1-4 chromosomal DNA (about 3×10-7 copies of the LeIF-like gene if single copy) and about 100 μpmol of each forward (N-terminal specific oligo) and reverse (C-terminal specific oligo) PCR primer. Higher than normal concentrations of primers (˜100 pmol/200 μl rxn) were used for amplification in order to compensate for any possible sequence variation between the PCR primers and the target gene sequence. This was necessary since the DNA sequence of the putative HP56 ORF determined by genomic sequencing may not be 100% accurate. In addition, an H. pylori strain different from that used for genomic sequencing was employed as the source of chromosomal DNA used to program subsequent PCR amplifications. Amplification of target sequences was achieved by heating the amplification reaction to 95° C. for ˜1.0 minute to fully denature chromosomal template DNA followed by a 32 cycle, three-step thermal amplification profile, i.e. 95° C., 45 sec; 60° C., 45 sec, 72° C., 1 min. Amplification was carried out in sealed 200 μl thin-walled polypropylene reaction tubes using a PE Biosystems Model 9700 thermal cycler. Following PCR amplification, an aliquot of the reaction (˜20 μl) was examined for the production of the appropriate 1.3 Kbp DNA fragment by agarose gel electrophoresis (0.8% agarose in a Tris-acetate-EDTA (TAE) buffer). A DNA molecular size standard (1 Kb DNA ladder, Life Technologies) was electrophoresed in parallel with PCR samples. Visualization of DNA in the gel was accomplished by ethidium bromide staining and ultraviolet illumination.

### 6.6. Cloning of the Hp56 PCR Product into the POE30 Expression Vector

The BamHI and SalI restriction sites engineered into the forward and reverse amplification primers, respectively, permitted directional cloning of the ˜1.3 Kbp PCR product into the commercially available E.coli expression plasmid pQE30 (Qiagen, ampicillin resistant) such that the HP56 protein could be expressed as a fusion protein containing a (His)6 affinity chromatography tag at the N-terminus. The 1.3 Kbp HP56 PCR product was purified from the amplification reaction using silica gel-based spin columns (Qiagen) according to the manufacturers instructions. To produce the required BamHI and SalI termini necessary for cloning, purified PCR product was sequentially digested to completion with BamHI and SalI restriction enzymes as recomnmended by the manufacturer (Life Technologies). Following the first restriction digestion, the PCR product was purified ia spin column as above to remove salts and eluted in sterile water prior to the second enzyme digestion. The digested DNA fragment was again purified using silica gel-based spin columns prior to ligation with the pQE30 plasmid. To prepare the expression plasmid pQE30 for ligation, it was similarly digested to completion with both BamHI and SalI and then treated with calf intestinal phosphatase (CIP, ˜0.02 units/pmole of 5′ end, Life Technologies) as directed by the manufacturer to prevent self ligation. A 5-fold molar excess of the digested fragment to the prepared vector was used to program the ligation reaction. A standard ˜20 ml ligation reaction (˜16° C., 16 hours) as described by Maniatis et al. (1982, Molecular Cloning: A Laboratory Manual, Cold Spring Harbor Laboratory Press, Cold Spring Harbor, N.Y.) was performed using T4 DNA ligase (˜2.0 units/reaction, Life Technologies). An aliquot of the ligation (˜5 ml) was used to transform electro-competent M15 (pREP4) cells using standard methodologies. Following a 2-3 hour outgrowth period at 37° C. in ˜1.0 ml of LB broth, transformed cells were plated on LB agar plates containing kanamycin (40 μg/ml) and ampicillin (100 μg/ml). Both antibiotics were included in the selection media to ensure that all transformed cells carried both the pREP4 plasmid (KnR), which carries the laclq gene necessary for IPTG-inducible expression of proteins on pQE30, and the pQE30-HP56 plasmid (ApR). Plates were incubated overnight at 37° C. for ˜16 hours. Individual KnR/ApR colonies were picked with sterile toothpicks and used to “patch” inoculate fresh LB KnR/ApR plates as well as a ˜1.0 ml LB KnR/ApR broth culture. Both the patch plates and the broth culture were incubated overnight at 37° C. in either a standard incubator (plates) or a shaking water bath.

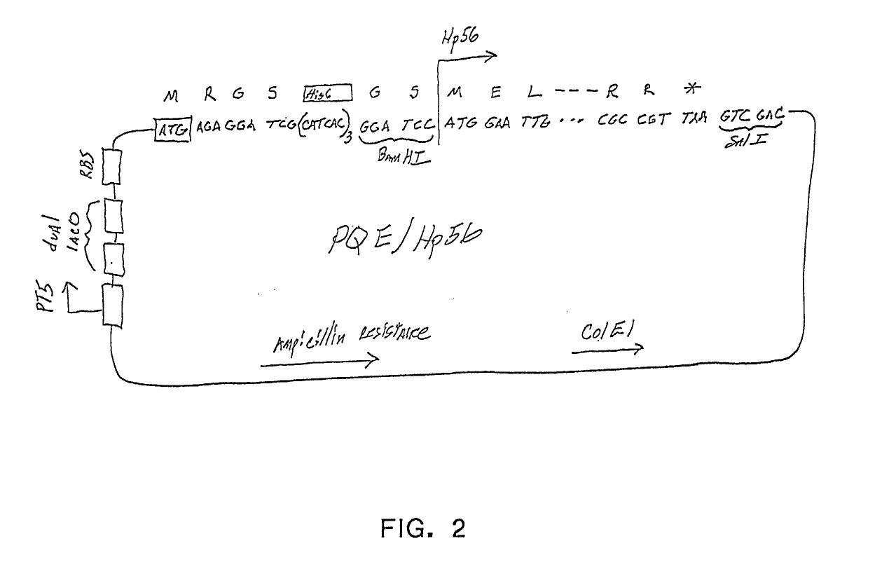

A whole cell-based PCR analysis was employed to verify that transformants contained the HP56 DNA insert. Here, the ˜1.0 ml overnight LB Kn/Ap broth culture was transferred to a 1.5 ml polypropylene tube and the cells collected by centrifugation in a Beckmann microcentrifuge (˜3 min., room temperature, ˜12K ×g). The cell pellet was suspended in ˜200 ml of sterile water and a ˜10 ml aliquot used to program a ˜50 ml final volume PCR reaction containing both HP56-Bam-F forward and HP56-Sal-RC reverse amplification primers. Final concentrations of the PCR reaction components were essentially the same as those specified in example 6.5. except ˜5.0 units of ampliTaq polymerase was used. The initial 95° C. denaturation step was increased to 3 minutes to ensure thermal disruption of the bacterial cells and liberation of plasmid DNA. An ABI Model 9700 thermal cycler and a 32 cycle, three-step thermal amplification profile, i.e. 95° C., 45 sec; 60° C., 45 sec, 72° C., 1 min., were used to amplify the HP56 fragment from the lysed transformant samples. Following thermal amplification, a ˜20 ml aliquot of the reaction was analyzed by agarose gel electrophoresis (0.8% agarose in a Tris-acetate-EDTA (TAE) buffer). DNA fragments were visualized by UV illumination after gel electrophoresis and ethidium bromide staining. A DNA molecular size standard (1 Kb ladder, Life Technologies) was electrophoresed in parallel with the test samples and was used to estimate the size of the PCR products. Transformants that produced the expected ˜1.3 Kbp PCR product were identified as strains containing a HP56 expression construct. A schematic map of the expression plasmid is shown in FIG. 2. Expression plasmid containing strains were then analyzed for the inducible expression of the Helicobacter pylori LeIF-like recombinant protein.

### 6.7. Expression Analysis of PCR-positive Transformants

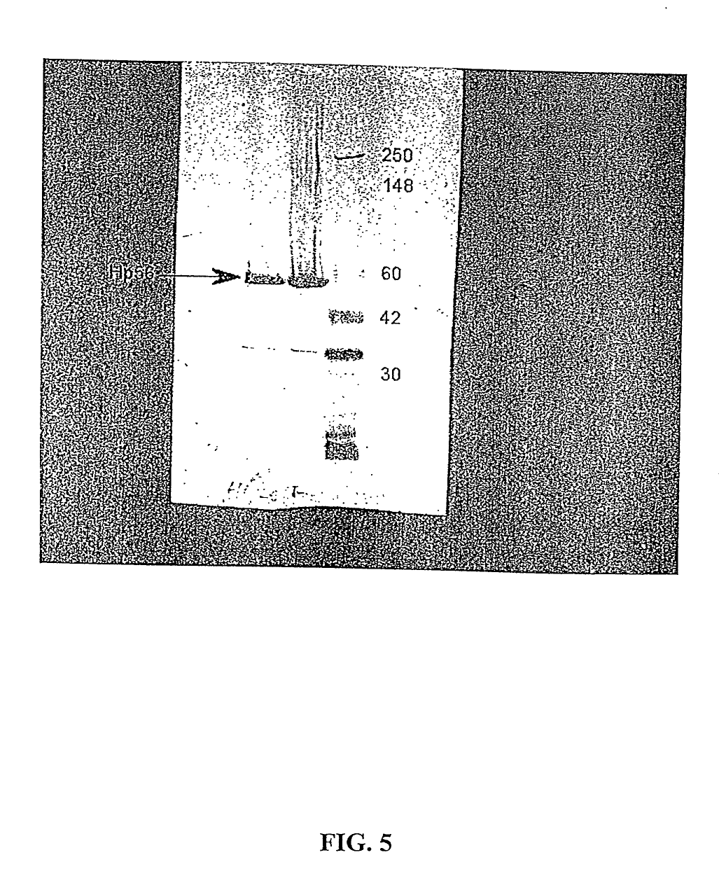

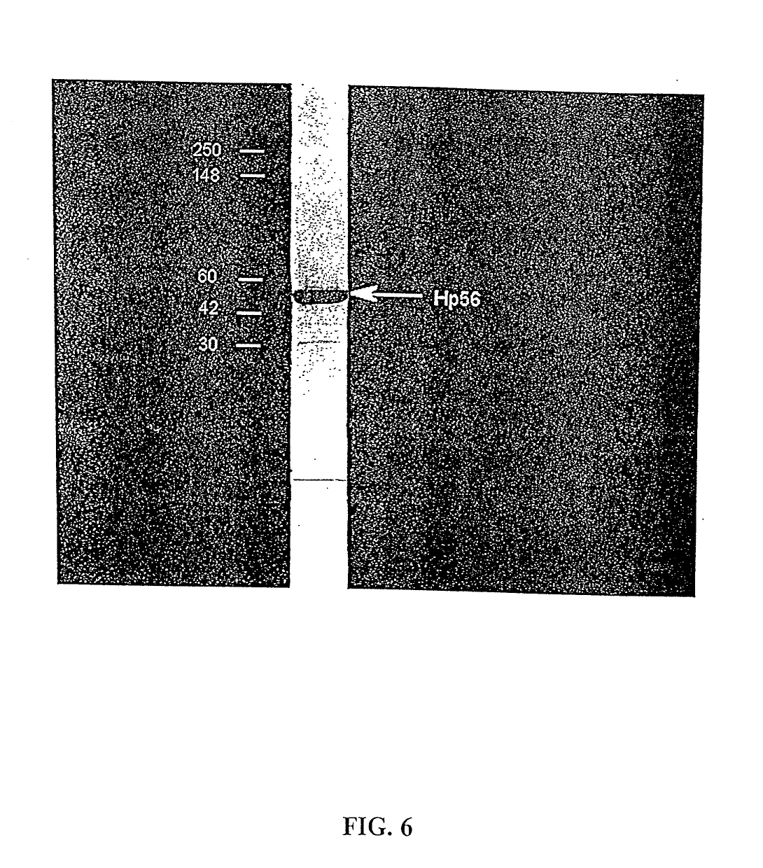

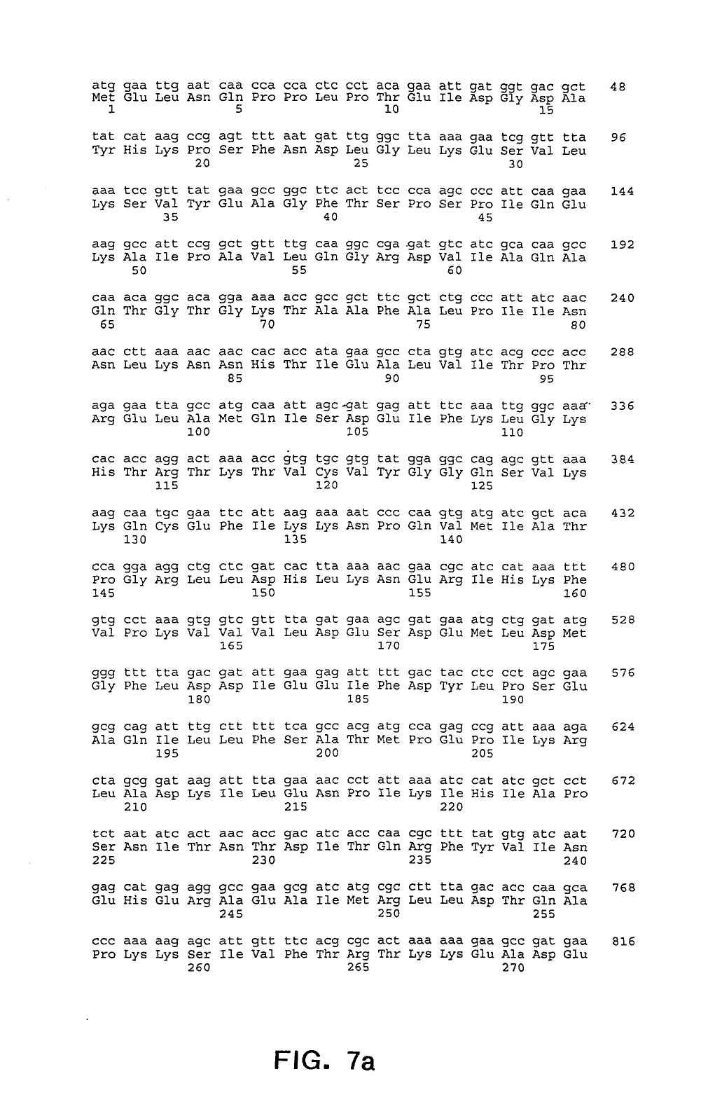

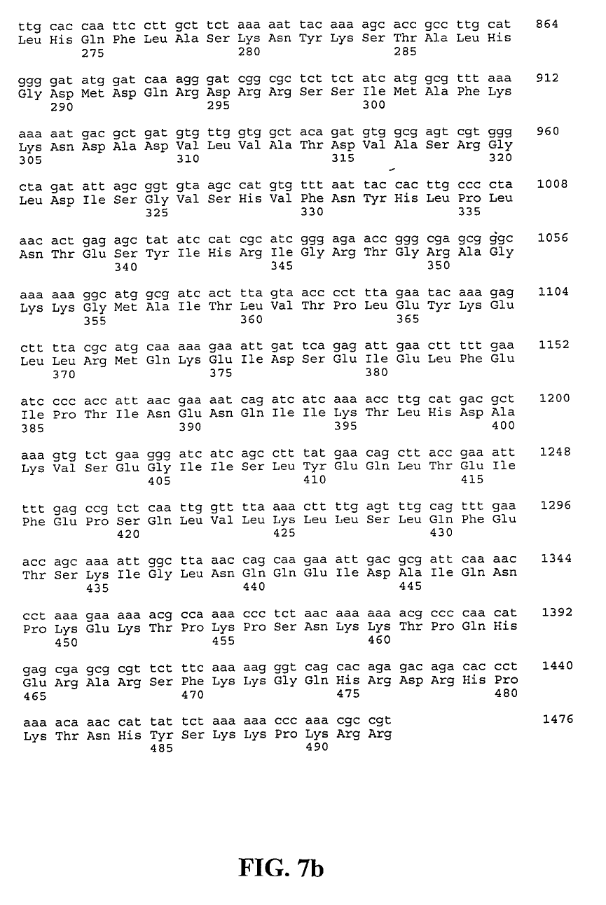

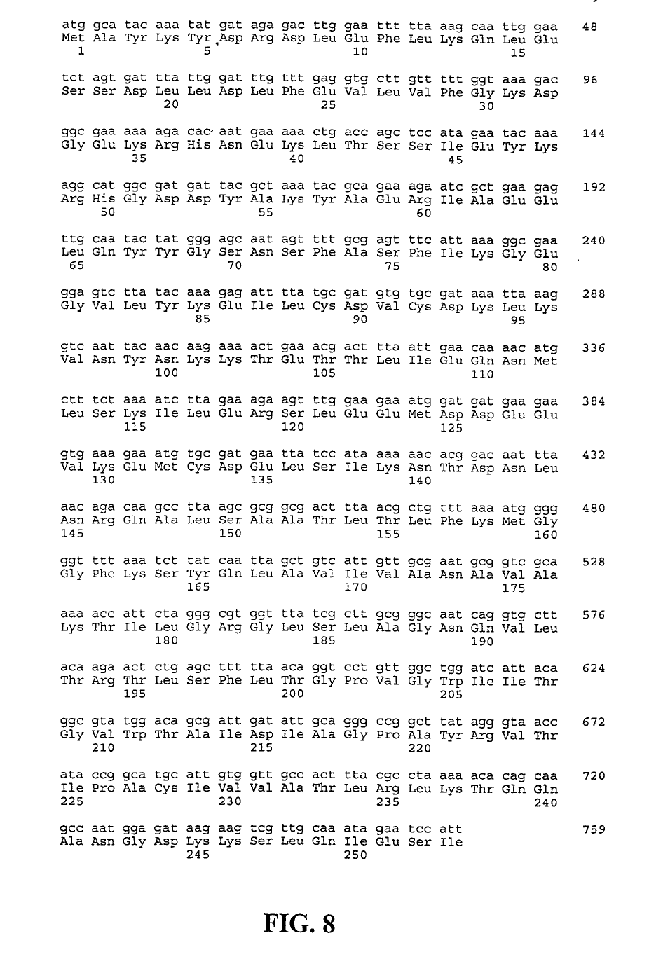

For each PCR-positive transformant identified above, ˜5.0 ml of LB broth containing kanamycin (40 mg/ml) and ampicillin (100 mg/ml) was inoculated with cells from the patch plate and grown overnight at 37° C. with shaking (˜250 rpm). An aliquot of the overnight seed culture (˜1.0 ml) was inoculated into a 125 ml Erlenmeyer flask containing ˜25 of LB Kn/Ap broth and grown at 37° C. with shaking (˜250 rpm) until the culture turbidity reached O.D.600 of ˜0.5, i.e. mid-log phase (usually about 1.5-2.0 hours). At this time approximately half of the culture (˜12.5 ml) was transferred to a second 125 ml flask and expression of recombinant Helicobacter pylori LeIF-like HP56 recombinant protein induced by the addition of IPTG (1.0M stock prepared in sterile water, Sigma) to a final concentration of 1.0 mM. Incubation of both the IPTG-induced and non-induced cultures continued for an additional ˜4 hours at 37° C. with shaking. Samples (1.0 ml) of both induced and non-induced cultures were removed after the induction period and the cells collected by centrifugation in a microcentrifuge at room temperature for ˜3 minutes. Individual cell pellets were suspended in ˜50 ml of sterile water, then mixed with an equal volume of 2×Lamelli SDS-PAGE sample buffer containing 2-mercaptoethanol, and placed in boiling water bath for 3 min to denature protein. Equal volumes (˜15 ml) of both the crude IPTG-induced and the non-induced cell lysates were loaded onto duplicate 12% Tris/glycine polyacrylamide gel (1 mm thick Mini-gels, Novex). The induced and non-induced lysate samples were electrophoresed together with prestained molecular weight markers (SeeBlue, Novex) under conventional conditions using a standard SDS/Tris/glycine running buffer (BioRad). Following electrophoresis, one gel was stained with Coomassie brilliant blue R250 (BioRad) and then destained with methanol:acetic acid:water (30%:10%:60%) to visualize novel approximately 50 kDa Helicobacter pylori LeIF-like recombinant protein (FIG. 6). The second gel was electroblotted onto a PVDF membrane (0.45 micron pore size, Novex) for ˜2 hrs at 4° C. using a BioRad Mini-Protean II blotting apparatus and Towbin's methanol (20%) transfer buffer. Blocking of the membrane and antibody incubations were performed using conventional methodologies. A monoclonal anti-RGS (His)6 antibody conjugated to HRP (QiaGen) was used at a 1/5,000 dilution to confirm the expression and identify of ˜50 kDa inducible protein(s) as a HP56 recombinant protein (FIG. 5). Visualization of the antibody reactive pattern was achieved on Hyperfilm using the Amersham ECL chemiluminescence system.

### 6.8. Production of Recombinant E.coli HP56 Cell Mass

A recombinant strain of E. coli M15 (pREP4) containing a recombinant plasmid encoding the LeIF-like gene from H. pylori was used to produce cell mass for purification of recombinant protein. The expression strain (E. coli M15pRE4PQE/HP56) was cultivated on LB agar plates containing 50 μg/ml kanamycin and 100 mg/ml ampicillin to ensure both the pREP4 lacIq control plasmid and the pQE30-HP56 ORF expression construct were both maintained. For cryopreservation at ˜80° C., the strain was propagated in LB broth containing the same concentration of antibiotics then mixed with an equal volume of LB broth containing 30% (w/v) glycerol.

The fermentation medium used for the production of recombinant protein consisted of 2XYT broth (Difco) containing 50 μg/ml kanamycin and 100 μg/ml ampicillin. Antifoam was added to medium for the fermenter at 0.25 ml/L (Antifoam 204, Sigma). To induce expression of HP56 recombinant protein, IPTG (Isopropyl-D-Thiogalactopyranoside) was added to the fermenter (1 mM, final concentration).

A 500-ml Erlenmeyer seed flask, containing 50 ml working volume, was inoculated with 0.3 ml of rapidly thawed frozen culture, or several colonies from a selective agar plate culture, and incubated for approximately 12 hours at 37±1° C. on a shaking platform at 150 rpm (Innova 2100, New Brunswick Scientific). This seed culture was then used to inoculate a 5-L working volume fermenter containing 2XYT broth and both Kn and Ap antibiotics. The fermenter (Bioflo 3000, New Brunswick Scientific) was operated at 37±1° C., 0.2-0.4 VVM air sparge, 250 rpm (2×yyy in Rushton impellers). pH was not controlled in either the flask seed culture or the fermenter. During fermentation, the pH ranged 6.5 to 7.3 in the fermenter. IPTG (1.0M stock, prepared in sterile water) was added to the fermenter when the culture reached mid-log of growth (˜0.7 O.D.600 units). Cells were induced for 2-4 hours then harvested by centrifugation using either a 28RS Heraeus (Sepatech) or RC5C superspeed centrifuge (Sorvall Instruments). Cell paste was stored at −20° C. until processed.

### 6.9. Identification of HP30 Open Reading Frame

Mice immunized with H. pylori cells (HWC) plus adjuvant, but not HWC alone, were protected from infection with Helicobacter infection when challenged with Helicobacter cells. Serum from mice vaccinated with H. pylori cell (HWC) plus adjuvant and serum from mice immunized with HWC alone were screened for reactivity on H. pylori cell lysate by Western Blot analysis. IgA antibody of serum from mice immunized with HWC plus adjuvant was reactive with a protein having an approximate molecular weight of 30 kDa. IgA antibody of serum from mice immunized with HWC alone was not reactive with the 30 kDa protein. Since the elicitation of immune responses to HP30 protein correlated with protection from infection, further characterization of HP30 protein was performed. The protein from the band on Western Blot was electoreluted and the N-terminal sequence determined using the methods described supra in Section 6.2. The Helicobacter pylori genomic sequence database (The Institute for Genomic Research, Rockville, Md.) was queried to identify an amino acid sequence with the N-terminal sequence of the 30 kDa protein. The protein is designated HP30.

### 6.10. PCR Amplification of HP30 ORF-specific DNA Fragments

The polymerase chain reaction (PCR) was employed to generate HP30 specific DNA fragments for expression cloning and genetic variability analysis. An N-terminal PCR forward primer was chemically synthesized that encodes the DNA sequence for the first ˜7 amino acids of the protein (ie the ˜21 nucleotide sequence beginning with the Met Translation initiation). In addition to the ORF-specific sequence the forward PCR primer also contained a short 5′ G/C claim (˜6 nucleotides) for efficient PCR amplification. A BamiHI restriction endonuclease cleavage site for use in subcloning was appropriately engineered into this primer between the G/C primer and the ORF-specific sequence.

The sequence of the HP30 N-terminal PCR forward strand primer is:5′-GCG GGA TCC ATG GCA TAC AAA TAT GAT AGA-3′ (SEQ ID NO:39).

An oligonucleotide having a DNA sequence complementary to the encoding the last ˜7 amino acids of the HP30 protein beginning with the endogenous stop codon (TAA) was synthesized and employed as a reverse PCR primer. Like the forward PCR primer, the reverse primer contained a short G/C clamp (˜6 nucleotide) for efficient DNA amplification and a SalI restriction endonuclease site appropriately positioned for subcloning. The sequence of the HP30-terminal PCR reverse strand primer is: 5′-GCG GTC GAC TTA AAT GGA TTC TAT TTG CAA CG-3′ (SEQ ID NO:40)

Oligonucleotides were synthesized on an Applied Biosystems Inc. (ABI) Model 380B DNA synthesizes using a 0.2 nmole scale column (ending mode trity-on, auto-cleavage) and standard phosphor-amidite chemistry. Crude oligonucleotides were manually purified over C18 reverse phase syringe columns (OPC column, ABI) as described by the manufacturer. Purity and yield were ascertained spectrophotometrically (230/260/280 ratios). Standard PCR amplification reactions (2mM Mg2+, 200 (mol dNTPs, 2.5 units recombinant AmpiTaq (PE Biosystems) in a 200 μl final reaction volume) were programmed using about 0.5 μg. H. pylori G1-4 chromosomal DNA and about 100 pmol of each forward (N-terminal specific oligo) and reverse (C-terminal specific oligo) PCR primer. Higher than normal concentrations of primers (˜100 pmol/200 (mol reaction) were used for amplification in order to compensate for any possible sequence variation between the PCR primers and the target gene sequence. This was necessary since the DNA sequence of the putative HP30 protein determined by genomic sequencing may not be 100% accurate. In addition, an H. pylori strain different from that used for genomic sequencing was employed as the source of chromosomal DNA used to program subsequent PCR amplifications. Amplification of target sequences was achieved by heating the amplification reaction to 95° C. for ˜1.0 minute to fully denature chromosomal template DNA followed by a 32 cycle, three-step thermal amplification profile, i.e. 95° C., 45 sec; 60° C., 45 sec, 72° C, 1 min. Amplification was carried out in sealed 200 μl thin-walled polypropylene reaction tubes using a PE Biosystems Model 9700 thermal cycler. Following PCR amplification, an aliquot of the reaction ˜20 μl was examined for the production of the appropriate DNA fragment by agarose gel electrophoresis (0.8% agarose in a Tris-acetate-EDTA (TAE) buffer). A DNA molecular size standard (1 Kb DNA ladder, Life Technologies) was electrophoresed in parallel with PCR samples. Visualization of DNA in the gel was accomplished by ethidium bromide staining and ultraviolet illumination.

### 6.11. Cloning of HP30 PCR Product onto Qe30 Expression Vector

The BamHI and SalI restriction sites engineered into the forward and reverse amplification primers, respectively, permitted directional cloning of the 1 Kbp PCR product into the commercially available E. coli expression plasmid pQE30 (Qiagen, ampicillin resistant) such that the HP30 protein could be expressed as a fusion protein containing a (His)6 affinity chromatography tag at the N-terminus. The 1 Kbp HP30 PCR product was purified from the amplification reaction using silica gel-based spin columns (Qiagen) according to the manufacturers instructions. To produce the required BamHI and SalI termini necessary for cloning, purified PCR product was sequentially digested to completion with BamHI and SalI restriction enzymes as recommended by the manufacturer (Life Technologies). Following the first restriction digestion, the PCR product was purified via spin column as above to remove salts and eluted in sterile water prior to the second enzyme digestion. The digested DNA fragment was again purified using silica gel-based spin columns prior to ligation with the pQE30 plasmid. To prepare the expression plasmid pQE30 for ligation, it was similarly digested to completion with both BamHI and SalI and then treated with calf intestinal phosphatase (CIP, ˜0.02 units/pmole of 5′ end, Life Technologies) as directed by the manufacturer to prevent self ligation. A 5-fold molar excess of the digested fragment to the prepared vector was used to program the ligation reaction. A standard ˜20 μl ligation reaction (˜16° C., ˜16 hours) as described by Maniatis et al. (1982, Molecular Cloning: A Laboratory Manual, Cold Spring Harbor Laboratory Press, Cold Spring Harbor, N.Y.) was performed using T4 DNA ligase (˜2.0 units/reaction, Life Technologies). An aliquot of the ligation (˜5 ul) was used to transform electro-competent M15 (pREP4) cells using standard methodologies. Following a 2-3 hour outgrowth period at 37° C. in ˜1.0 ml of LB broth, transformed cells were plated on LB agar plates containing kanamycin (40 μg/ml) and ampicillin (100 μg/ml). Both antibiotics were included in the selection media to ensure that all transformed cells carried both the pREP4 plasmid (KnR), which carries the laclq gene necessary for IPTG-inducible expression of proteins on pQE30, and the pQE-HP30 plasmid (ApR). Plates were incubated overnight at 37° C. for ˜16 hours. Individual KnR/ApR colonies were picked with sterile toothpicks and used to “patch” inoculate fresh LB KnR/ApR plates as well as a ˜1.0 ml LB KnR/ApR broth culture. Both the patch plates and the broth culture were incubated overnight at 37° C. in either a standard incubator (plates) or a shaking water bath.

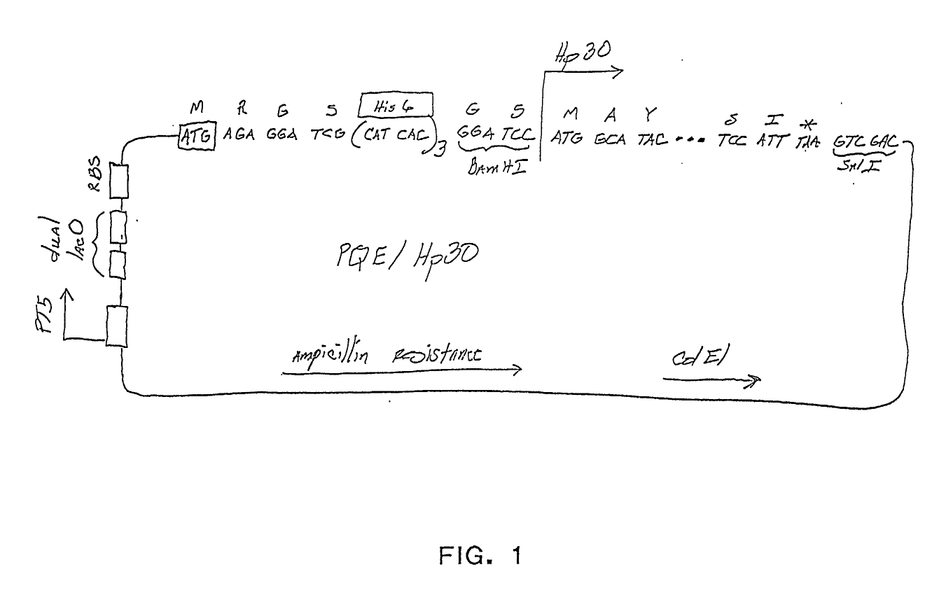

A whole cell-based PCR analysis was employed to verify that transformants contained the HP30 DNA insert. Here, the ˜1.0 ml overnight LB Kn/Ap broth culture was transferred to a 1.5 ml polypropylene tube and the cells collected by centrifugation in a Beckmann microcentrifuge (˜3 min., room temperature, ˜12K×g). The cell pellet was suspended in ˜200 μl of sterile water and a 10 ml aliquot used to program a ˜50 μl final volume PCR reaction containing both HP56-Bam-F forward and HP56-Sal-RC reverse amplification primers. Final concentrations of the PCR reaction components were essentially the same as those specified in example 6.1 except ˜5.0 units of ampliTaq polymerase was used. The initial 95° C. denaturation step was increased to 3 minutes to ensure thermal disruption of the bacterial cells and liberation of plasmid DNA. An ABI Model 9700 thermal cycler and a 32 cycle, three-step thermal amplification profile, i.e. 95° C., 45 sec; 60° C., 45 sec, 72° C., 1 min., were used to amplify the HP30 fragment the lysed transfornant samples. Following thermal amplification, a ˜20 μl aliquot of the reaction was analyzed by agarose gel electrophoresis (0.8% agarose in a Tris-acetate-EDTA (TAE) buffer). DNA fragments were visualized by UV illumination after gel electrophoresis and ethidium bromide staining. A DNA molecular size standard (1 Kb ladder, Life Technologies) was electrophoresed in parallel with the test samples and was used to estimate the size of the PCR products. Transformants that produced the expected ˜1. Kbp PCR product were identified as strains containing a HP30 expression construct. A schematic map of the HP30 expression plasmid is shown in FIG. 1. Expression plasmid containing strains were then analyzed for the inducible expression of the Helicobacter pylori HP30 recombinant protein.

### 6.12. Expression Analysis of HP30 PCR-positive Transformants

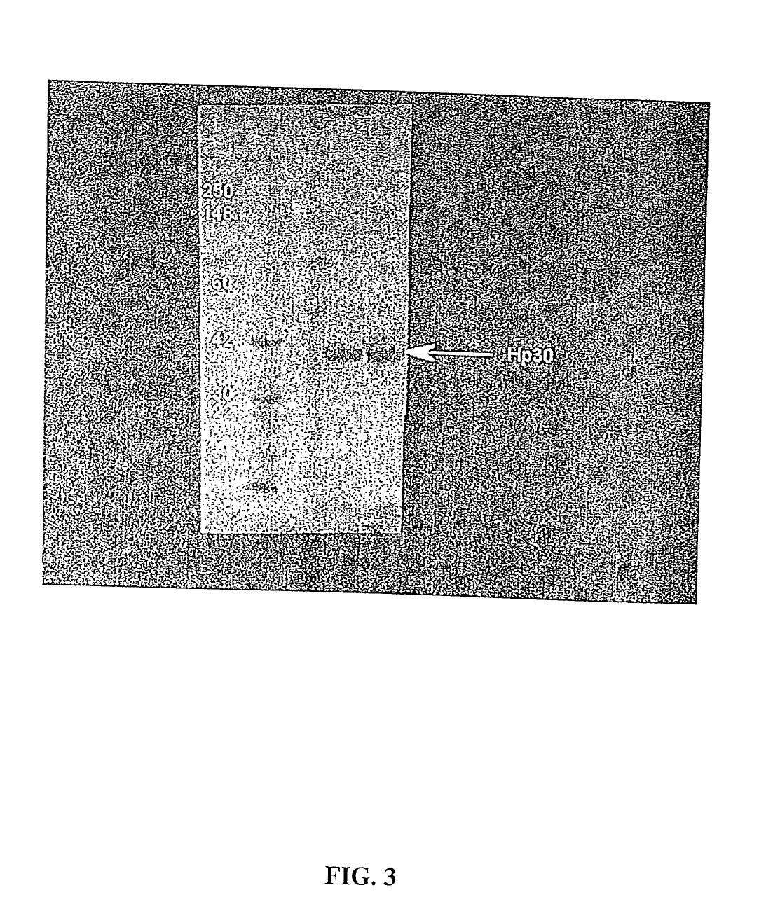

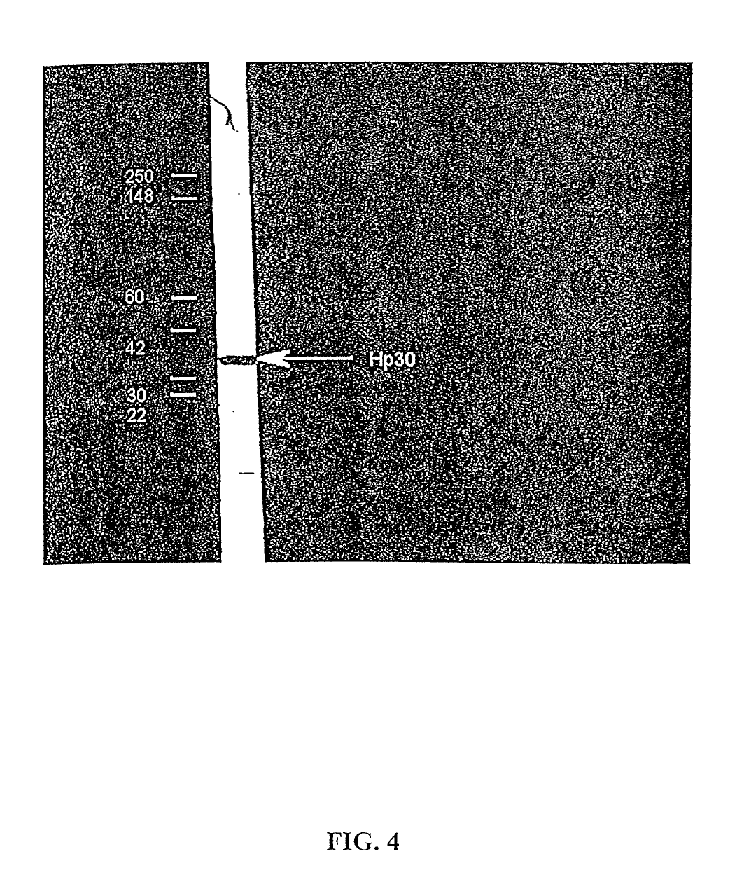

For each PCR-positive transformant identified above, ˜5.0 ml of LB broth containing kanamycin (40 μg/ml) and ampicillin (100 μg/ml) was inoculated with cells from the patch plate and grown overnight at 37° C. with shaking (˜250 rpm). An aliquot of the overnight seed culture (˜1.0 ml) was inoculated into a 125 ml Erlenmeyer flask containing ˜25 of LB Kn/Ap broth and grown at 37° C. with shaking (˜250 rpm) until the culture turbidity reached O.D. 600 of ˜0.5, i.e. mid-log phase (usually about 1.5-2.0 hours). At this time approximately half of the culture (˜12.5 ml) was transferred to a second 125 ml flask and expression of recombinant Helicobacter pylori HP30 recombinant protein induced by the addition of IPTG (1.0M stock prepared in sterile water, Sigma) to a final concentration of 1.0 mM. Incubation of both the IPTG-induced and non-induced cultures continued for an additional ˜4 hours at 37° C. with shaking. Samples (˜1.0 ml) of both induced and non-induced cultures were removed after the induction period and the cells collected by centrifugation in a microcentrifuge at room temperature for ˜3 minutes. Individual cell pellets were suspended in ˜50 μl of sterile water, then mixed with an equal volume of 2×Lamelli SDS-PAGE sample buffer containing 2-mercaptoethanol, and placed in boiling water bath for ˜3 min to denature protein. Equal volumes (˜15 μl) of both the crude IPTG-induced and the non-induced cell lysates were loaded onto duplicate 12% Tris/glycine polyacrylamide gel (1 mm thick Mini-gels, Novex). The induced and non-induced lysate samples were electrophoresed together with prestained molecular weight markers (SeeBlue, Novex) under conventional conditions using a standard SDS/Tris/glycine running buffer (BioRad). Following electrophoresis, one gel was stained with Coomassie brilliant blue R250 (BioRad) and then destained with methanol:acetic acid:water (30%:10%:60%) to visualize novel ˜30 kDa Helicobacter pylori recombinant protein (FIG. 4). The second gel was electroblotted onto a PVDF membrane (0.45 micron pore size, Novex) for ˜2 hrs at 4° C. using a BioRad Mini-Protean II blotting apparatus and Towbin's methanol (20%) transfer buffer. Blocking of the membrane and antibody incubations were performed using conventional methodologies. A monoclonal anti-RGS (His)6 antibody conjugated to HRP (QiaGen) was used at a 1/5,000 dilution to confirm the expression and identify of ˜30 kDa inducible protein(s) as a HP30 recombinant protein (FIG. 3). Visualization of the antibody reactive pattern was achieved on Hyperfilm using the Amersham ECL chemiluminescence system.

### 6.13. Production of Recombinant E. coli HP-30 Cell Mass

A recombinant strain of E. coli M15(pREP4) containing a recombinant plasmid encoding the gene encoding 30 kDa protein from H. pylori was used to produce cell mass for purification of recombinant protein. The expression strain (M15pRE4PQE/HP30) was cultivated on LB agar plates containing 50 μg/ml kanamycin and 100 μg/ml ampicillin to ensure both the pREP4 lacIq control plasmid and the pQE-HP30 ORF expression construct were both maintained. For cryopreservation at −70° C., the strain was propagated in LB broth containing the same concentration of antibiotics then mixed with an equal volume of LB broth containing 30% (w/v) glycerol.

The fermentation medium used for the production of recombinant protein consisted of 2 XYT broth (DIFCO) containing 50 μg/ml kanamycin and 100 mg/ml ampicillin. Antifoam was added to medium for the fermenter at 0.25 ml/L (Antifoam 204, Sigma). To induce expression of HP30 recombinant protein, IPTG (Isopropyl β,-D-Thiogalactopyranoside) was added to the fermenter (1 mM, final concentration).

A 500-ml Erlenreyer seed flask, containing 50 ml working volume, was inoculated with 0.3 ml of rapidly thawed frozen culture, or several colonies from a selective agar plate culture, and incubated for approximately 12 hours at 37±1° C. on a shaking platform at 150 rpm (Innova 2100, New Brunswick Scientific). This seed culture was then used to inoculate a 5-L working volume fermenter containing 2XYT broth and both Kn and Ap antibiotics. The fermenter (Bioflo 3000, New Brunswick Scientific) was operated at 37±1° C., 0.2-0.4 VVM air sparge, 250 rpm (2×yyy in Rushton impellers). pH was not controlled in either the flask seed culture or the fermenter. During fermentation, the pH ranged 6.5 to 7.3 in the fermenter. IPTG (1.0M stock, prepared in sterile water) was added to the fermenter when the culture reached mid-log of growth (˜0.7 O.D. 600 units). Cells were induced for 2-4 hours then harvested by centrifugation using either a 28RS Heraeus (Sepatech) or RC5C superspeed centrifuge (Sorvall Instruments). Cell paste was stored at −20° C. until processed.

### 6.14. Purification of the HP056 and HP-30 Recombinant Protein

Approximately 15 gm of frozen cell paste was resuspended by vortexing and trituration in ˜40 ml of ice cold 50 mM sodium phosphate buffer (pH8.0), 10 mM Tris-Hcl (pH8.0), 100 mM NaCl and disrupted by passage through a Niro-Soavi high pressure homogenizer according to manufacturers recommendations (˜5 ml/min flow rate, ˜450 bars). The cell lysate was then centrifuged for 5 min at ˜500×g (4° C.) in a Sorvall SS34 rotor to remove unbroken cells.

The cleared homogenate was then mixed with 3-5 ml of Ni-NTA Sepharose immobilized metal affinity chromatography (IMAC) resin (QiaGen), transferred to a 250 ml sterile Erlenmeyer flask, placed on a platform rotator, and recombinant HP56 or HP30 containing an N-terminal (His)6 affinity tag allowed to bind to the resin for 16 hrs at 4° C. Following batch binding, the homogenate-resin slurry was transferred to a conventional glass chromatography column (BioRad Econo Column) and the lysate allowed to drain out. Unbound, contaminating proteins were removed from the resin by slowly washing the column with 2-100 ml volumes of wash buffer (50 mM NaH₂PO₄, pH7.0; 100 mM NaCl).

Recombinant, affinity tagged HP56 or HP30 bound to the resin was eluted in 2 ml volumes using an imidazole-based elution buffer (20 mM Tris-Hcl, pH8.0; 100 mM NaCl; 100-200 mM imidazole). Aliquots of each elution fraction were analyzed by SDS-PAGE using 4-20% Tris-Glycine gradient gels (Novex) and a commercially prepared pre-stained molecular weight marker set (MultiMark, Novex, San Diego, Calif.). Following electrophoresis, the gel was stained with a Coomassie blue R250 solution (BioRad) and destained to visualize eluted proteins. Fractions where the recombinant HP56 or HP30 protein was>80% pure were pooled and dialyzed overnight (˜14-18 hours) in a commercial dialysis cassette (MWCO=10 kDa; Slidalyzer, Pierce Chem.) against a Tris-HCl (pH7.3) to remove residual imidazole and salt. Dialyzed eluent was then concentrated by ultrafiltration using a 30 kDa spin concentrator (Centricon-30; Amicon).

The protein concentration of the concentrated HP56 or HP30 was determined using the Micro BCA method (Pierce Chem.) and BSA as a standard. Purified HP56 or HP30 (˜1.0 mg/ml protein concentration) was evaluated for purity, identity, and residual endotoxin burden by SDS-PAGE, Western blot, and a colorimetric endotoxin assay (BioWhittaker), respectively. The gel-purified HP56 or HP30 material displayed a purity of >80% as a single band of the expected molecular size (˜50 kDa or 30 kDa, respectively) by gel analysis and reacted vigorously with anti-RGS-(His)6 antibody in Western blots. Residual endotoxin was calculated to be<0.05 EU/μg.

### 6.15. Properties of Hp30 and Hp56

HP30 polypeptide exists as a protein of approximately 30 kDa in its native state as determined via Western blots of extracts of H. pylori. HP56 polypeptide exists as a protein of approximately 56 kDa in its native state as determined via Western blots of extracts of H. pylori.

### 6.16. Anti-Hp30 or Anti-Hp56 Antiserum

Antisera to HP30 or HP56 was prepared by injecting the HP30 or HP56 polypeptide into an animal, such as a rabbit, mouse or guinea pig, with or without an adjuvant by any methods generally known to those skilled in the art. For instance, HP30 was injected into a rabbit with Freund's complete adjuvant followed by injection of HP30 with Freund's incomplete adjuvant. Normally, a serni-purified or purified form of the protein is injected. For instance, the HP30 polypeptide is resolved from other proteins using a denaturing sodium dodecylsulfate polyacrylamide gel according to standard techniques well known to those skilled in the art, as previously described (Laemmli, 1970, Nature 227:680-685), and cutting the HP30-containing band out of the gel. The excised band containing HP30 is macerated and injected into an animal to generate antiserum to the polypeptide. Alternatively, the rHP30 or rHP56 was purified as described supra and injected into animals. The antisera were examined using well known and generally accepted methods of ELISA to determine titer, by Western blots to determine binding to proteins, for immunofluorescent staining and for complement-mediated cytotoxic activity against Helicobacter.

### 6.17. Elisa

Anti-HP30 or anti-HP56 antibody titers were measured by ELISA using purified HP30 or HP56 protein (˜1 μg /well), Alternatively, H. pylori (whole cell preparation or crude cell lysate) were used as capture ligands by any methods known by those skilled in the art. Serial dilutions of antisera were made in PBS and tested by ELISA in duplicate. HRP-conjugated antibody diluted is used as the second reporter antibody in these assays. Titers were expressed as the greatest dilution showing positive ELISA reaction, ie an O.D.450 value>2SD above the mean negative control value (e.g. prebled rabbit sera).

### 6.18. Western Blots

H. pylori were grown as describe in section 6.1 and H. pylori lysates were prepared. Alternatively, lysates of E.coli harboring plasmids encoding HP56 or HP30 were prepared. The solubilized cells were resolved on 4-12% polyacrylarnide gels as per Laemmli and the separated proteins were electrophoretically transferred to PVDF membranes at 100 V for 1.5 hours as previously described (Thebaine et al., 1979, Proc. Natl. Acad. Sci. USA 76:4350-4354). The PVDF membranes were then pretreated with 25 ml of Dulbecco's phosphate buffered saline containing 0.5% sodium casein, 0.5% bovine serum albumin and 1% goat serum. All subsequent incubations were carried out using this pretreatment buffer.

PVDF membranes were incubated with 25 ml of a dilution of preimmune serum or serum from an animal immunized with HP30 or HP56 polypeptide (as described above) for 1 hour at room temperature. PVDF membranes were then washed twice with wash buffer (20 mM Tris buffer [pH 7.5.] containing 150 mM sodium chloride and 0.05% Tween-20). PVDF membranes were incubated with 25 ml peroxidase-labeled goat anti-rabbit (or anti-mouse for murine antibodies) immunoglobulin (eg. anti-IgG or anti-IgA) (Jackson ImmunoResearch Laboratories, West Grove, Pa.) for 30 minutes at room temperature. PVDF membranes were then washed 4 times with wash buffer, and were developed with 3,3′ diaminobenzidine tetra-hydrochloride and urea peroxide as supplied by Sigma Chemical Co. (St. Louis, Mo. catalog number D-4418) for 4 minutes each.

Hyperimmune antisera or murine antibody (including but not limited to serum from immunized mice or monoclonal antibodies) were used to probe Western blots of crude H. pylori extracts as well to identify proteins reactive with antisera generated against HP30 or HP56 protein.

### 6.19. Urease Assay

Animal tissue was placed in 0.5 ml of urease test solution (0.0468% NaH₂PO₄₂H₂O, 0.0007% Phenol red, 2.4%Urea) for approximately 4 hours incubation (H. felis) or approximately 24 hours (H. pylori). The presence of a pink color indicates the presence of urease in the test sample.

### 6.20. H. pylory Colonization Assay

After challenge with H. pylori or H. felis, the stomach of mice was removed and rinsed in PBS to remove food particles. The stomach was split longitudinally with a razor blade, weighed and homogenized. Serial dilutions of the stomach were made and plated on selective media. After 7 days incubation at 37° C., the plates were removed and colonies were counted.

### 6.21. Vaccine Efficacy Mouse Model of H. pylory Infection

Helicobacter-free mice were employed to evaluate the efficacy of HP30 and HP56 to protect animals against H. pylori infection. For prophylactic studies, the test group of mice was vaccinated orally by first administering 0.5 ml 5% sodium bicarbonate followed 10 minutes later with 0.25 ml of vaccine with or without adjuvant in PBS. Oral vaccinations were administered three times at day 0, day 14 and day 28. Intranasal vaccinations were administered by administering HP30, HP56 or Helicobacter whole cell (HWC) with or without adjuvant in PBS. Subcutaneous injection of vaccines was administered by injecting each mouse at two subcutaneous sites (eg back of the neck and abdomen) on days 0, 21 and 35. Two weeks after the last vaccination (therapeutic), mice were challenged by intragastric inoculation of one dose of 10⁷ H. felis or 3 doses of 18 H. pylori given within a 5 day period. For therapeutic studies, mice were first colonized (Day 0) with H. felis or H. pylori as described above and then orally vaccinated on Days 21, 35 and 49 after challenge, intranasally vaccinated with a single dose on Day 21 or subcutaneously vaccinated on Days 21, 42 and 56. Two weeks after challenge (prophylactic) or after the last vaccination (therapeutic), mice were euthanized with CO₂ and the longitudinal section of the entire stomach (H. pylori) or half of the antrum (H. felis) removed. For determination of urease activity, the stomach was assayed as described in Section 6.19. For quantitation of H. pylori, the stomach was assayed as described in Section 6.20.

The ability of rHP30 and rHP56 to act as therapeutic agents to decrease or eliminate H. pylory colonization in mice previously infected with H. pylori is shown in Table 3. Historically mice colonized with live H. pylori have approximately 10⁶ to 10⁷ CFU/ml. Mice were colonized with live H. pylori as described supra on Day 0 and subcutaneously vaccinated on Days 21, 42 and 56 with either rHp30, rHP56 or both rHP30 and rHP56 with adjuvant (Alum, CFA or Alum +AB5). Mice vaccinated with either rHP30 or rHP56 or both rHP30 and rHP56 using alum as an adjuvant had reduced levels of Helicobacter. When mice were immunized with rHP30 and rHP56 using alum and AB5 as an adjuvant Helicobacter infection was completely eliminated. Helicobacter infection was also completely eliminated in mice vaccinated with rHP30 or rHP56 in CFA as an adjuvant.

TABLE 3
Therapeutic treatment of mice colonized with Helicobacter pylori by
subcutaneous vaccination with rHP30 or rHP56

| | Urease Test | |
|---|---|---|
| Vaccine | Positive/Total | Stomach Culture CFU/ml |
| rHP30 + Alum | 1/5 | 1 × 10⁴ |
| rHP56 + Alum | 1/5 | 4 × 10⁴ |
| rHP30 + rHP56 | 2/5 | 1 × 10⁵ |
| rHP30 + rHP56 + Alum | 0/5 | 1 × 10³ |
| rHP30 + rHP56 + alum + | 0/5 | 0 |
| AB5 | | |
| rHP30 + CFA | 0/5 | 0 |
| rHP56 + CFA | 0/5 | 0 |

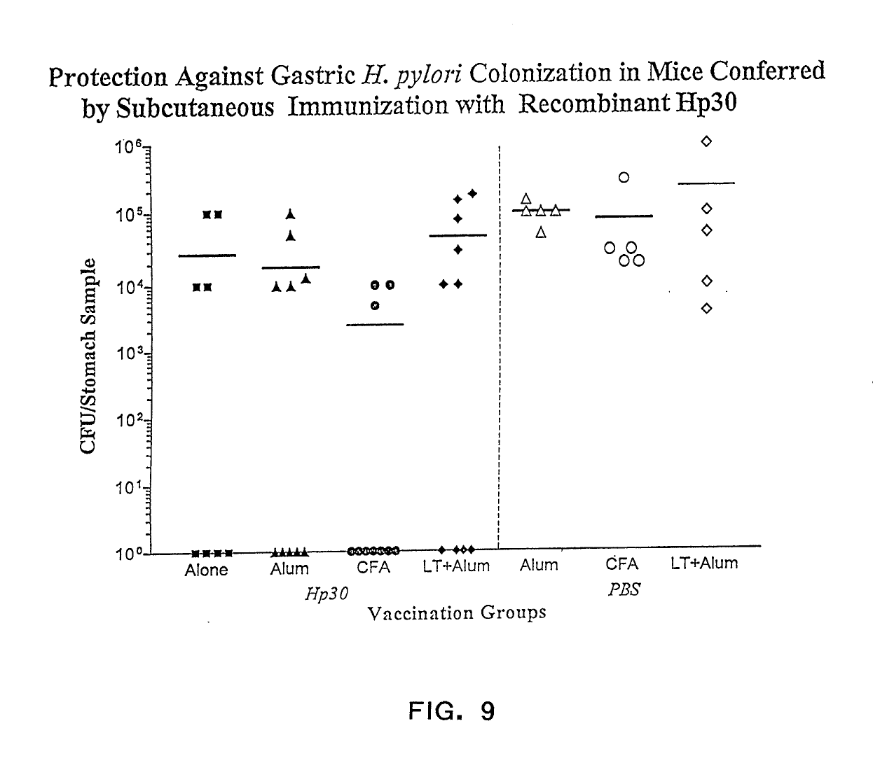

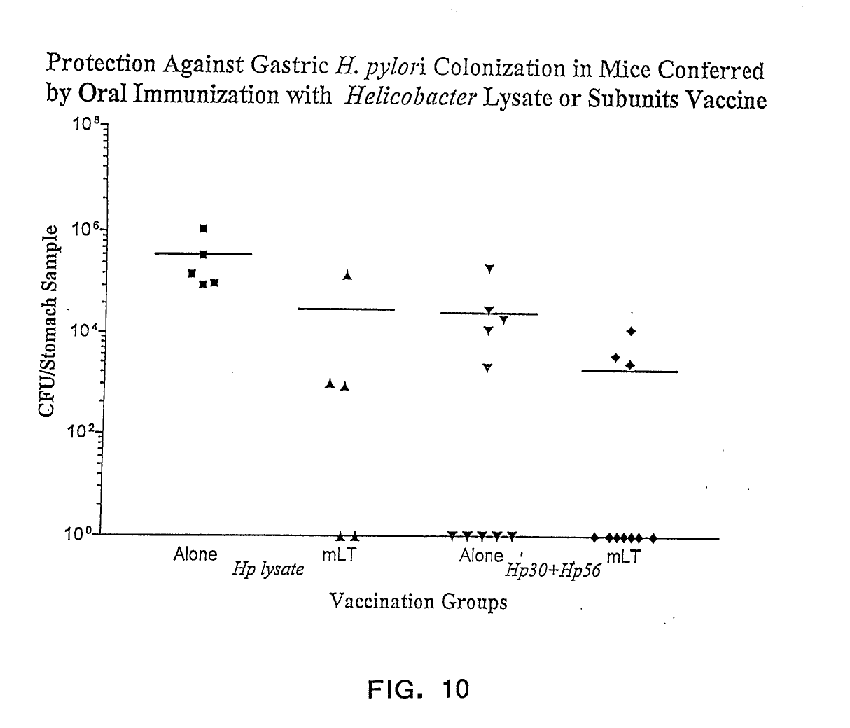

The ability of rHP30 or rHP56 to protect mice from subsequent infection with H. pylori is shown in Table 4 and FIGS. 9 and 10. As shown in Table 4, mice vaccinated by nasal vaccination with rHP30 or rHP56 using AB5 as an adjuvant were not protected against colonization with Helicobacter. However, mice vaccinated intranasally with both rHP30 and rHP56 and the adjuvant AB5 were protected against colonization when challenged with Helicobacter.

The data in FIG. 9 clearly demonstrate that≧50% of animals vaccinated subcutaneously with recombinant HP30 are protected against subsequent H. pylori gastric colonization or are colonized at lower levels than control mice. These results also demonstrate that the protective efficacy of the HP30 antigen can be achieved by subcutaneous immunization with or without the co-administration of parenteral adjuvants.

The data in FIG. 10 clearly demonstrate that≧50% of animals vaccinated orally with recombinant HP30 and HP56 are protected against subsequent H. pylori gastric colonization and that the remaining animals are colonized at lower levels than mice immunized with crude H. pylori cell lysate. These results also demonstrate that the protective efficacy of the HP30 and HP56 antigens can be achieved with or without the co-administration of an adjuvant.

TABLE 4
Protection against Helicobacter colonization by Intranasal vaccination
with rHP30 or rHP56

| Vaccine | % Protection* |
|---|---|
| rHP30 + AB5 | 0 |
| rHP56 + AB5 | 0 |
| rHP30 + rHP56 + AB5 | 100 |

*Protection determined by the number of animals negative in urease test 21 days after challenge with H felis.

To determine rHP30 or rHP56 anti-Helicobacter humoral responses, blood samples are collected periodically during the immunization and challenge phases by retroorbital bleeding and serum prepared by centrifugation. Quantitation of antibody (Ab) responses by ELISA are performed as described in Section 6.17. Microwell ELISA plates (Maxisorb, NUNC) for determining antibody levels are coated overnight at 4° C. with ˜0.5-10 (g of purified rHP30 or rHP56 or H. pylori whole cell (˜6×108 cells per well) in 10 mM carbonate/bicarbonate buffer (pH 9.6), washed with PBS containing 0.1% Tween-20 (washing buffer) and blocked for ˜1hr at 37° C. with a PBS solution containing 3% BSA. For the determination of antigen-specific serum IgG levels, test sera are serially diluted in washing buffer containing 0.5% BSA and aliquots (100 μl incubated in the antigen-coated wells for ˜2hr at 37° C. The plates are then washed and incubated for ˜1 hr at 37° C. with a horseradish peroxidase (HRP)-conjugated goat anti-mouse IgG second antibody (Sigma). An HRP-conjugated goat anti-mouse IgA secondary antibody is used to detect the presence of HP30 or HP56 specific IgA. After incubation with the appropriate secondary antibody, the plates are washed and incubated for ˜20-30 minutes at room temperature with TMB substrate (Sigma). Reactions are stopped by the addition of 2M H₂SO₄ and the absorbance determined at 450 nm on a microplate reader. Titers are determined as the reciprocal of the sample dilution corresponding to an optical density of 1.0 at 450 nm.

Anti-Helicobacter IgG antibody responses in mice subcutaneously vaccinated with rHP30, rHP56 or rHP30 and rHP56 using Alum, Alum plus AB5, or CFA as adjuvant are shown in Table 5. Mice immunized with rHP30 and rHP56 using Alum as an adjuvant had the same high titer of anti-Helicobacter IgG antibody as mice immunized with HP30 or HP56 using CFA as an adjuvant. Mice immunized with rHP30 and rHP56 using Alum and AB5 as adjuvants had a lower IgG antibody titer.

TABLE 5
Antibody responses to H. pylori whole cell lysate induced by
subcutaneous vaccination with recombinant HP30 or HP56

| VACCINE | IgG response* |
|---|---|
| rHP30 | 9,000 |
| rHP30 + Alum | 40,000 |
| rHP56 + Alum | 14,000 |
| rHP30 + rHP56 | 20,000 |
| rHP30 + rHP56 + Alum | 390,000 |
| rHP30 + rHP56 + Alum + AB5 | 65,000 |
| rHP30 + AB5 | 46 |
| rHP56 + AB5 | 30 |
| rHP30 + rHP56 + AB5 | 33,938 |
| rHp30 + CFA | 390,000 |
| rHP56 + CFA | 390,000 |

*Antibody response expressed as titer in ELISA using H. pylori whole cell as capture antigen

### Determination of Specific Cellular Responses to HP30 or HP56

Groups of mice are immunized with a vaccine comprising rHP30 and/or rHP56 and optionally an adjuvant. For instance, mice are immunized with HP30 and adjuvant. Seven days after last immunization, animals from each group are sacrificed by CO₂ asphyxiation, spleens removed and single cell suspensions prepared using conventional methodologies. Spleen cells from immunized animals are analyzed separately or spleens from 2 animals are pooled. For both the positive control group (sham immunized and sham infected) and the negative control group (sham immunized, infected) spleen cells are pooled and tested for restimulation.

For the measurement of spleen cell proliferation, spleens are ground (5 to 10 rounds) in 5 ml of RPMI 1640 Glutamax I supplemented with 10% fetal calf serum, 25 mM HEPES, 50 U/ml penicillin, 50 μg /ml streptomycin, 1 mm sodium pyruvate, nonessential amino acids, and 50 M 2- mercaptoethanol (Gibco-BRL). Live cells are counted by Trypan Blue staining and diluted in the same media to reach a density of 1.0-2.0×10⁶ cells/ml (Falcon 2063 polypropylene tubes). Triplicate cultures are set-up in round bottom 96-well culture plates (Nunclon, Nunc) using ˜5×10⁵ responder cells per well in 200 μl of media. Cells are stimulated with either 1.0 μg /ml of rHP30 or rHP56 (antigen-specific proliferation) or with 4 μg/ml concanavalin A (Boerhinger Mannheim) as a positive stimulation control; unrestimulated cell cultures are used as a negative control of cellular activation. After 72-96 hours of incubation at 37° C. in 5% CO₂ cells are pulsed labeled for 18 hrs with 1.0 Ci ³H-thymidine (Amersham) per well. Pulsed cells are harvested onto glass-fiber sheets using a Tomtec Cell Harvester (Mk III) and counted for beta-emission in a 3-channel Wallac 1450 Trilux Liquid Scintillation Counter. The stimulation index (SI) for a sample (individual or pooled) is defined as the mean of the antigen or ConA-stimulated T-cell uptake of ³H-thymidine for triplicate wells divided by the mean of the unstimulated uptake for triplicate wells. Sls for both antigen-specific (rHP30 or rHP56 -specific) and ConA-specific proliferation are determined.

For measurement of cytokine levels, spleen and lymph node cells were harvested 10 days after the last vaccination. The cells were stimulated with the appropriate antigens and supernatants were collected at 24, 48 and 72 hours incubation. Cytokine levels were measured with a sandwich ELISA kit (Endogen, Woburn, Mass.). Units of cytokine production were determined by comparing the absorbance at 405 nm from stimulated cells to standard curve.

### 6.22. Evaluation of Adjuvant Activity

The Helicobacter felis antrum colonization model was employed to evaluate the adjuvant effects of recombinant HP56. Four groups of female Balb/C mice (˜6 weeks of age, Jackson Labs) were employed for this evaluation. One group of 5 animals received two intranasal doses of a vaccine composed of a formalin-inactivated Helicobacter pylori whole cell (HWC) antigen (˜1.0×10⁹ HWC particles) and ˜10 ug of the recombinant, purified HP56 (˜30 ml total volume, in sterile PBS). Two groups of 5 female mice per group were immunized similarly; one group received a preparation containing only the HP56 prototype adjuvant (Doug, no whole cell antigen) while the other group received a vaccine consisting of the HWC antigen (˜1.0×10⁹ particles) together with ˜5 μg of a modified form of the E.coli heat-labile toxin (mLT) as a control mucosal adjuvant. The fourth group of 5 animals served as a null adjuvant control and were immunized with a vaccine composed of the inactivated HWC antigen and ˜10 μg of a recombinant protein having no adjuvant activity. Immunizations were given 14 days apart. Prior to immunization, mice were sedated using an anesthesia cocktail consisting of 16% Ketaject and 16% Xylaject in 68% pyrogen-free PBS (100 ml i.p./animal). Sedated animals were placed on their backs and using a standard laboratory pipette administered the vaccine formulation; ˜10 μl of the vaccine solution per nostril.

Approximately 10 days after the second immunization, blood was collected by retroorbital bleeding and sera prepared by centrifugation. Individual serum IgG and IgA titers directed to either the HWC test antigen or to the HP56 prototype adjuvant were determined by ELISA. Microwell ELISA plates (Maxisorb, NUNC) for determining antibody (Ab) levels were coated overnight at 4° C. with ˜0.5-1.0 μg of either the inactivated HWC antigen or recombinant HP56 per well in 10 mM carbonate/bicarbonate buffer (pH9.6). Once coated with capture antigen, microtiter plates were washed with PBS containing 0.1% Tween-20 (washing buffer) and blocked for ˜1 hr at 37° C. with a PBS solution containing 3% BSA. For the determination of serum IgG and IgA levels, test sera were serially diluted in washing buffer containing 0.5% BSA and aliquots (100 μl) incubated in the antigen-coated wells for ˜2 hr at 37° C. The plates were then washed and incubated for ˜1 hr at 37° C. with a horseradish peroxidase (HRP)-conjugated goat anti-mouse IgG second antibody (Sigma). A HRP-conjugated goat anti-mouse IgA secondary antibody was used to detect the presence of HWC specific IgA in vaginal secretions. After incubation with the appropriate secondary antibody, the plates were washed and incubated for ˜20-30 min at room temperature with TMB substrate (Sigma). Reactions were stopped by the addition of 2M H₂SO₄ and the absorbance determined at 450 nm on a Molecular Devices SpectroMax microplate reader. Titers were determined as the reciprocal of the sample dilution corresponding to an optical density of 1.0 at 450 nm. As noted in Table 6, below, intranasal administration of the recombinant HP56 stimulated the production of HWC-specific serum IgA and IgG levels approximately 10-fold and 35-fold, respectively.

TABLE 6
Adjuvant Activity of Recombinant HP56

| | HWC IgA | HWC IgG | HP56 IgA | HP56 IgG |
|---|---|---|---|---|
| Sample | Titer | Titer | Titer | Titer |
| rHWC + | 3770 | 65610 | 15 ± 11 | 28782 ± 21870 |
| HP56 | | | | |
| PBS + | 16 ± 36 | 10 ± 0 | 72 ± 34 | 31247 ± 50208 |
| HP56 | | | | |
| HWC + | 7290 ± 0 | 81732 ± 58683 | 10 ± 0 | 10 ± 0 |
| AB5 | | | | |
| HWC + | 43 ± 69 | 1757 ± 2697 | 10 ± 0 | 4209 ± 2806 |
| mSL Tiiv | | | | |

### 6.23. Generation of a Radiolabelled Screening Probe

The sequence information shown above is used to design a pair of ondegenerate oligonucleotide primers. PCR amplification of DNA fragments is performed under the same conditions as described above with the exception that the annealing temperature is lowered to 50° C. The DNA fragment is isolated from an agarose gel as before and radiolabeled using [³²P]-gamma-ATP and T4 polynucleotide kinase according to standard methods. Unincorporated radiolabel is separated from the probe on a G25 Sepharose spin column. Before use, the probe is denatured for 2 min. at 95° C. described above with the exception that the annealing temperature is lowered to 50° C. and subsequently chilled on ice (4° C.).

### 6.24. Hybiridization of Plaque-lift Filters and Southern Blots with Radiolabelled Probe

Phage plaques from library platings are immobilized on nylon filters using standard transfer protocols well known to those skilled in the art. Digested bacterial genomic DNA, phage or plasmid DNA is electrophoresed on 0.8% TAE-agarose gels and transferred onto nylon filters using a pressure blotter (Stratagene) according to the manufacturer's recommendations. Hybridizations with selected probes are performed at 37° described above with the exception that the annealing temperature is lowered to 50° C. Hybridizations with other probes are generally carried out at 60° described above with the exception that the annealing temperature is lowered to 50° C. Washes of increasing stringency are done at the respective hybridization temperatures until nonspecific background is minimized.

### 6.25. Construction of a H. Pylori Fenomic DNA Library

A genomic library is constructed in the λZAPII replacement vector obtained from Stratgene. The vector arms is digested with EcoR1. Digests of H.pylori DNA by EcoR1 is performed to yield fragment sizes between 1 kb and 5 kb. Ligations of vector arms and insert DNA is carried out according to standard protocols. Ligation reactions are packaged in vitro using the Stratagene GigaPack Gold III extract. The packaged phage are plated on E. coli X1 Blue MRA (P2) (Stratagene). An initial library titer is determined and expressed as number of pfu.

The library is screened using 4×10⁴ pfu that are plated at a density of 8×10³ pfu/130 mm plate. Several putative positive phage plaques are located and the strongest hybridizing phage are eluted from cored agarose plugs, titered and replated for secondary screening. The selected phages are replated at low density (approximately 100 pfu/plate) and plaques are analyzed by PCR using primer pairs. Inserts carrying plasmids (phagemids) are rescued from the selected phage by co-infecting E. coli cells with an appropriate helper virus.

### 6.26. Determination of Insert Size and Mapping of DNA Fragments

In order to estimate the size of inserts, phagemid DNA is digested with NotI and the digests are analyzed on a 0.5% TAE-agarose gel side by side with suitable DNA markers. In order to map restriction fragments that would hybridize to the probe, DNA from phagemid isolates is digested with a number of common restriction enzymes either alone or in combination with NotI. The rationale of this approach is to discriminate between fragments that span the insert/phagemid vector junction and those that map on the NotI insert. The series of single and double digests are run side-by-side for each phage isolate and analyzed by Southern analysis with radiolabeled probe.

### 6.27. Sequencing of the Hp30 or Hp56 Gene

Sequencing of the nucleic acid encoding rHP30 or rHP56 is performed using the Dye Terminator Cycle Sequencing Kit from Perkin-Elmer according to the manufacturer's specifications. The sequencing reactions are read using an ABI Prism 310 Genetic Analyzer. The sequences are aligned using the AutoAssembler software (Perkin-Elmer) provided with the ABI Prism 310 sequencer.

The present invention is not to be limited in scope by the microorganism deposited or the specific embodiments described herein. It will be understood that variations which are functionally equivalent are within the scope of this invention. Indeed, various modifications of the invention, in addition to those shown and described herein, will become apparent to those skilled in the art from the foregoing description and accompanying drawings. Such modifications are intended to fall within the scope of the appended claims.

Various publications are cited herein, the disclosures of which are incorporated by reference in their entireties.

What is claimed is:

1. An isolated HP30 or HP56 polypeptide of Helicobacter spp, wherein the HP30 has a molecular weight of 30 kDa and the HP56 kDa has a molecular weight of 56 kDa as determined in SDS polyacrylamide gel electrophoresis.

2. The HP30 or HP56 polypeptide of claim 1, wherein the Helicobacter spp. is selected from the group consisting of Helicobacter pylori and Helicobacter felis.

3. The HP30 or HP56 polypeptide of claim 2, wherein the Helicobacter spp is Helicobacter pylori.

4. The HP56 or HP30 polypeptide of claim 1, comprising sequence SEQ ID NO: 2 or 4, a fragment thereof, a sequence encoded by a nucleic acid molecule comprising the sequence SEQ ID NO: 1 or 3, a fragment thereof, or a sequence encoded by a nucleic acid molecule which hybridizes to a nucleic acid molecule comprising the sequence of SEQ ID NO: 1 or 3 under high stringency conditions comprising (a) prehybridization of filters with DNA at 50° C. in buffer comprised of 6×SSC, 50 mM Tris-HCl (pH 7.5), 1 mM EDTA, 0.02% PVP, 0.02% Ficoll, 0.02% BSA and 500 mg/ml denatured salmon sperm DNA; (b) hybridization at 65° C. in prehybridization mixture containing 100 mg/ml denatured salmon sperm DNA and (c) washing of filters at 37° C. in solution containing 2×SSC, 0.01% PVP, 0.01% Ficoll 0.01% BSA, wherein said fragment is at least 6 amino acids in length.

5. The HP56 or HP30 polypeptide of claim 1 or an at least 6 amino peptide fragment thereof, which specifically binds an antibody that specifically binds to a protein having the sequence selected from the group consisting of SEQ ID NOs: 2 and 4.

6. A peptide fragment of the HP30 or HP56 polypeptide of claim 1, wherein said peptide fragment is at least 6 amino acids in length.

7. The peptide fragment of claim 6 wherein said fragment comprises the sequence of SEQ ID NO: 5-20.

8. An isolated fusion polypeptide comprising at least two peptides, each of said peptides selected from the group of peptides having an amino acid sequence selected from sequences consisting of the SEQ ID NOS:5, 6, 7, 8, 9, 10, 11, 12, 13, 14, 15, 16, 17, 18, 19 and 20 with the proviso that the peptides are arranged in a configuration that is different from naturally occurring configuration.

9. The isolated fusion polypeptide of claim 8 wherein the fusion polypeptide comprises SEQ ID NO: 5, 6, 7, 8, 9, 10 and 11 with the proviso that the peptides of said fusion polypeptide are arranged in a configuration that is different from naturally occurring configuration.

10. The isolated fusion polypeptide of claim 8 wherein the fusion polypeptide comprises SEQ ID NO: 16, 17, 18, 19, and 20, with the proviso that the peptides of said fusion polypeptide are arranged in a configuration that is different from naturally occurring configuration.

11. An antibody or an antigen-binding fragment thereof that specifically binds the HP56 or HP30 polypeptide of claim 1.

12. An antibody or an antigen-binding fragment thereof that specifically binds the peptide fragment of claim 6.

13. An antibody or an antigen-binding fragment thereof that specifically binds a peptide fragment having an amino acid sequence selected from the group consisting of SEQ ID NO:5, 6, 7, 8, 9, 10, 11, 12, 13, 14, 15, 16, 17, 18, 19, and 20.

14. The antibody of claim 11, 12 or 13 which is a cytotoxic, cytostatic, or neutralizing antibody.

15. A vaccine composition comprising the HP30 or HP56 polypeptide of claim 1 or comprising the HP30 polypeptide and HP56 polypeptide of claim 1 and a pharmaceutically acceptable carrier or diluent.

16. The vaccine of claim 15 further comprising one or more adjuvants or immunostimulatory compounds.

17. The vaccine of claim 16 wherein the adjuvants or immunostimulatory compounds are selected from the group consisting of alum, mLT, QS21, MF59, CpG DNA, PML, calcium phosphate and PLG.

18. The vaccine of claim 16 comprising one adjuvant or immunostimulatory compound.

19. The vaccine of claim 16 comprising two different adjuvants or immunostimulatory compounds.

20. A vaccine comprising the peptide fragment of claim 6 and a pharmaceutically acceptable carrier or diluent.

21. A vaccine of claim 20 further comprising one or more adjuvants or immunostimulating compounds.

22. The vaccine of claim 21 wherein the one or more adjuvants or immunostimulatory compounds are selected from the group consisting of alum, mLT, QS21, MF59, CpG DNA, PML, calcium phosphate and PLG.

23. The vaccine of claim 21 comprising one adjuvant or immunostimulatory compound.

24. The vaccine of claim 21 comprising two different adjuvants or immunostimulatory compounds.

25. A vaccine comprising the isolated fusion polypeptide of claim 8 and a pharmaceutically acceptable carrier or diluent.

26. The vaccine of claim 25 further comprising one or more adjuvants or immunostimulatory compounds.

27. The vaccine of claim 26 wherein the one or more adjuvants or immunostimulatory compounds are selected from the group consisting of alum, mLT, QS21, MF59, CpG DNA, PML, calcium phosphate and PLG.

28. The vaccine of claim 26 comprising one adjuvant or immunostimulatory compound.

29. The vaccine of claim 26 comprising two different adjuvants or immunostimulatory compounds.

30. A vaccine comprising the antibody of claim 11 and a pharmaceutically acceptable carrier or diluent.

31. The vaccine any one of claims 15, 20 or 25 additionally comprising one or more immunogens selected from the group consisting of lipids, lipoproteins, phospholipids, lipooligosaccharides, proteins, attenuated organisms and inactivated whole cells.

32. The vaccine of claim 15, 20 or 25 additionally comprising one or more immunogens selected from the group consisting of H. pylori cytotoxin, H. pylori hsp60, H. pylori CagA, H. pylori urease, H. pylori catalase, H. pylori nickel biding protein, H. pylori tagA, H. pylori enolase, entire attenuated or killed organisms or subunits therefrom of Campylobacter spp., Shigella spp., Enteropathogenic E. coli spp, Vibrio cholera or rotavirus.

33. An isolated nucleic acid molecule comprising a nucleotide sequence encoding an isolated HP30 or HP56 polypeptide or an at least 6 amino acid fragment thereof, of Helicobacter spp, wherein the HP30 has a molecular weight 30 kDa and HP56 has a molecular weight of 56 kDa as determined in SDS polyacrylamide gel electrophoresis or fragment thereof.

34. An isolated nucleic acid molecule having the sequence of SEQ ID NO: 1 or 3, an at least 18 nucleotide fragment thereof, or the complement thereof.

35. A pharmaceutical composition comprising the isolated nucleic acid molecule of claim 33.

36. A vaccine comprising the isolated nucleic acid molecule of claim 33.

37. A vaccine comprising an isolated nucleic acid encoding the HP30 or HP56 polypeptide of claim 1 or an at least 18 nucleotide fragment thereof, and further comprising one or more adjuvants or immunostimulatory compounds which may be the same or different.

38. The vaccine of claim 37 wherein the one or more adjuvants or immunostimulatory compounds are selected from the group consisting of alum, MLT, QS21, MF59, CpG DNA, PML, calcium phosphate and PLG.

39. The vaccine of claim 37 comprising one adjuvant or immunostimulatory compound.

40. The vaccine of claim 37 comprising two different adjuvants or immunostimulatory compounds.

41. A vaccine comprising one or more of an isolated HP30 or HP56 polypeptide of Helicobacter spp. wherein the HP30 has a molecular weight of 30 kDa and HP56 kDa has a molecular weight of 56 kDa as determined in SDS of polyacrylamide gel electrophoresis; or an isolated nucleic acid comprising a nucleotide sequence encoding an HP30 or HP56 polypeptide Helicobacter spp. wherein the HP30 has a molecular weight of 30 kDa and HP56 kDa has a molecular weight of 56 kDa as determined in SDS of polyacrylamide gel electrophoresis said vaccine further comprising one or more adjuvants or immunostimulatory compounds selected from the group consisting of alum, mLT, QS21, MF59, CpG, DNA, PML, calcium phosphate and PLG.

42. A method of producing an immune response in an animal comprising administering to the animal an immunogenic amount of the HP30 or HP56 polypeptide of claim 1.

43. A method of producing an immune response in an animal comprising administering to the animal an immunogenic amount of the peptide fragment of claim 6.

44. A method of producing an immune response in an animal comprising administering to the animal an immunogenic amount of the isolated fusion polypeptide of claim 8.

45. A method of producing an immune response in an animal comprising administering to the animal an immunogenic amount of the nucleic acid molecule of claims 33 or 34.

46. A method of producing an immune response in an animal comprising administering to the animal an immunogenic amount of the vaccine of claim 41.

47. A method of producing an immune response in an animal comprising administering to the animal an immunogenic amount of one or more vaccines of claims 15, 20, 25, 30, 36, 37 or 41, wherein said vaccine are administered simultaneously or sequentially.

48. Plasmid M15 (PRE4)PQE/HP30 obtainable from E. coli, as deposited with the ATCC and assigned accession number PTA-2670.

49. Plasmid M15(PRE4)PQE/HP56 obtainable from E. coli, as deposited with the ATCC and assigned accession number PTA-2669.

50. A recombinant expression vector adapted for transformation of a host comprising the nucleic acid molecule of claim 33 or 34.

51. The recombinant expression vector of claim 50 further comprising an expression means operatively coupled to the nucleic acid molecule for expression by the host of HP30 or HP56 protein or an at least 6 amino acid fragment thereof.

52. The expression vector of claim 51, wherein the expression means includes a nucleic acid portion encoding a sequence for purification of the HP30 or HP56 protein.

53. The expression vector of claim 51 wherein the expression means further includes a nucleic acid portion that directs secretion from the host of the HP30 or HP56 polypeptide.

54. A transformed host cell containing an expression vector of claim 50.

55. The transformed host cell containing the plasmid of claim 48 or 49.

56. A host cell containing the nucleic acid molecule of claim 33 or 34 operatively linked to a heterologous promoter.

57. An isolated recombinant HP30 or HP56 polypeptide of Helicobacter spp. produced by a method comprising culturing the transformed host cell of claim 54 under conditions suitable for expression of said HP30 or HP56 polypeptide and recovering said HP30 or HP56 polypeptide.

58. An isolated recombinant HP30 or HP56 polypeptide produced by a method comprising culturing the transformed host cell of claim 55 under conditions suitable for expression said HP30 or HP56 polypeptide and recovering said HP30 or HP56 polypeptide.

59. An isolated recombinant HP30 or HP56 polypeptide produced by a method comprising culturing the transformed host cell of claim 56 under conditions suitable for expression of said HP30 or HP56 polypeptide and recovering said HP30 or HP56 polypeptide.

60. A method of preventing, treating or ameliorating a disorder or disease associated with infection of an animal with Helicobacter by administering an effective amount of the polypeptide of claim 1.

61. A method of preventing, treating or ameliorating a disorder or disease associated with infection of an animal with Helicobacter by administering an effective amount of the polypeptide fragment of claim 6.

62. A method of preventing, treating or ameliorating a disorder or disease associated with infection of an animal with Helicobacter by administering an effective amount of the isolated fusion polypeptide of claim 8.

63. A method of preventing, treating or ameliorating a disorder or disease associated with infection of an animal with Helicobacter by administering an effective amount of the vaccine of claim 30.

64. A method of preventing, treating or ameliorating a disorder or disease associated with infection of an animal with Helicobacter by administering an effective amount of the vaccine of claim 31.

65. A method of preventing, treating or ameliorating a disorder or disease associated with infection of an animal with Helicobacter by administering an effective amount of the vaccine of claim 32.

66. A method of preventing, treating or ameliorating a disorder or disease associated with infection of an animal with Helicobacter by administering an effective amount of the vaccine of claim 37.

67. A method of preventing, treating or ameliorating a disorder or disease associated with infection of an animal with Helicobacter by administering an effective amount of the vaccine of claim 41.

68. A method of preventing, treating or ameliorating a disorder or disease associated with infection of an animal with Helicobacter by administering an effective amount of the vaccine of claim 15.

69. A method of preventing, treating or ameliorating a disorder or disease associated with infection of an animal with Helicobacter by administering to a subject in need of such prevention, treatment or amelioration, an effective amount of one or more vaccines of claims 15, 20, 25, 30, 31, 36 or 41, each optionally comprising one or more immunogens selected from the group consisting of a lipid, lipoprotein, phospholipid, lipoligosaccharide, protein, attenuated organism and inactivated whole cell, wherein said vaccines are administered simultaneously or sequentially.

70. The method of claim 69 which further comprises administering one or more antibiotics which has Helicobacter bactericidal activity wherein said antibiotic is administered prior to, simultaneously, or sequentially to the administration of said one or more vaccines.

71. The method of claim 70 wherein in said one or more antibiotics is selected from the group consisting of meprazole, clarithromycin, omeprazole, metronidazole, tetracycline, Lansoprazole and amoxicillin.

72. An antagonist which inhibits the activity or expression of the polypeptide of claim 1.

73. An antagonist which inhibits the expression of the nucleic acid of claim 33.

74. A method for identifying compounds which interact with the polypeptide of claim 1, said method comprising contacting a composition comprising said polypeptide with the compound to be screened under conditions to permit interaction between the compound and the polypeptide and detecting the interaction of the compound with the polypeptide.

75. A method for identifying compounds which interact with an activity of the nucleic acid molecule of claim 33, said method comprising contacting a composition comprising the nucleic acid with the compound to be screened under conditions to permit interaction between the compound and the nucleic acid and detecting the interaction of the compound with the nucleic acid.

76. A method of preventing, treating or ameliorating a disorder or disease associated with infection of an animal with Helicobacter by administering to a subject in need of such prevention, treatment or amelioration, an effective amount of one or more vaccines of claim 32, each optionally comprising one or more immunogens selected from the group consisting of a lipid, lipoprotein, phospholipid, lipoligosaccharide, protein, attenuated organism and inactivated whole cell, wherein said vaccines are administered simultaneously or sequentially.

77. The method of claim 76 which further comprises administering one or more antibiotics which has Helicobacter bactericidal activity wherein said antibiotic is administered prior to, simultaneously, or sequentially to the administration of said vaccine.

78. The method of claim 77 wherein in said one or more antibiotics is selected from the group consisting of meprazole, clarithromycin, omeprazole, metronidazole, tetracycline, Lansoprazole and amoxicillin.


SEQUENCE LISTING (44 sequences)

SEQ ID NO: 1 - DNA, 1476 bp, Helicobacter sp.
atggaattgaatcaaccaccactccctacagaaattgatggtgacgcttatcataagccgagttttaatgatttgggcttaaaagaatcggttttaaaatccgtttatgaagccggcttcacttccccaagccccattcaagaaaaggccattccggctgttttgcaaggccgagatgtcatcgcacaagcccaaacaggcacaggaaaaaccgccgctttcgctctgcccattatcaacaaccttaaaaacaaccacaccatagaagccctagtgatcacgcccaccagagaattagccatgcaaattagcgatgagattttcaaattgggcaaacacaccaggactaaaaccgtgtgcgtgtatggaggccagagcgttaaaaagcaatgcgaattcattaagaaaaatccccaagtgatgatcgctacaccaggaaggctgctcgatcacttaaaaaacgaacgcatccataaatttgtgcctaaagtggtcgttttagatgaaagcgatgaaatgctggatatggggtttttagacgatattgaagagatttttgactacctccctagcgaagcgcagattttgcttttttcagccacgatgccagagccgattaaaagactagcggataagattttagaaaaccctattaaaatccatatcgctccttctaatatcactaacaccgacatcacccaacgcttttatgtgatcaatgagcatgagagggccgaagcgatcatgcgccttttagacacccaagcacccaaaaagagcattgttttcacgcgcactaaaaaagaagccgatgaattgcaccaattccttgcttctaaaaattacaaaagcaccgccttgcatggggatatggatcaaagggatcggcgctcttctatcatggcgtttaaaaaaaatgacgctgatgtgttggtggctacagatgtggcgagtcgtgggctagatattagcggtgtaagccatgtgtttaattaccacttgcccctaaacactgagagctatatccatcgcatcgggagaaccgggcgagcgggcaaaaaaggcatggcgatcactttagtaacccctttagaatacaaagagcttttacgcatgcaaaaagaaattgattcagagattgaactttttgaaatccccaccattaacgaaaatcagatcatcaaaaccttgcatgacgctaaagtgtctgaagggatcatcagcctttatgaacagcttaccgaaatttttgagccgtctcaattggttttaaaacttttgagtttgcagtttgaaaccagcaaaattggcttaaaccagcaagaaattgacgcgattcaaaaccctaaagaaaaaacgccaaaaccctctaacaaaaaaacgccccaacatgagcgagcgcgttctttcaaaaagggtcagcacagagacagacaccctaaaacaaaccattattctaaaaaacccaaacgccgt

SEQ ID NO: 2 - protein, 492 aa, Helicobacter sp.
MELNQPPLPTEIDGDAYHKPSFNDLGLKESVLKSVYEAGFTSPSPIQEKAIPAVLQGRDVIAQAQTGTGKTAAFALPIINNLKNNHTIEALVITPTRELAMQISDEIFKLGKHTRTKTVCVYGGQSVKKQCEFIKKNPQVMIATPGRLLDHLKNERIHKFVPKVVVLDESDEMLDMGFLDDIEEIFDYLPSEAQILLFSATMPEPIKRLADKILENPIKIHIAPSNITNTDITQRFYVINEHERAEAIMRLLDTQAPKKSIVFTRTKKEADELHQFLASKNYKSTALHGDMDQRDRRSSIMAFKKNDADVLVATDVASRGLDISGVSHVFNYHLPLNTESYIHRIGRTGRAGKKGMAITLVTPLEYKELLRMQKEIDSEIELFEIPTINENQIIKTLHDAKVSEGIISLYEQLTEIFEPSQLVLKLLSLQFETSKIGLNQQEIDAIQNPKEKTPKPSNKKTPQHERARSFKKGQHRDRHPKTNHYSKKPKRR

SEQ ID NO: 3 - DNA, 759 bp, Helicobacter sp.
atggcatacaaatatgatagagacttggaatttttaaagcaattggaatctagtgatttattggatttgtttgaggtgcttgtttttggtaaagacggcgaaaaaagacacaatgaaaaactgaccagctccatagaatacaaaaggcatggcgatgattacgctaaatacgcagaaagaatcgctgaagagttgcaatactatgggagcaatagttttgcgagtttcattaaaggcgaaggagtcttatacaaagagattttatgcgatgtgtgcgataaattaaaggtcaattacaacaagaaaactgaaacgactttaattgaacaaaacatgctttctaaaatcttagaaagaagtttggaagaaatggatgatgaagaagtgaaagaaatgtgcgatgaattatccataaaaaacacggacaatttaaacagacaagccttaagcgcggcgactttaacgctgtttaaaatggggggttttaaatcttatcaattagctgtcattgttgcgaatgcggtcgcaaaaaccattctagggcgtggtttatcgcttgcgggcaatcaggtgcttacaagaactctgagctttttaacaggtcctgttggctggatcattacaggcgtatggacagcgattgatattgcagggccggcttatagggtaaccataccggcatgcattgtggttgccactttacgcctaaaaacacagcaagccaatggagataagaagtcgttgcaaatagaatccatt

SEQ ID NO: 4 - protein, 253 aa, Helicobacter sp.
MAYKYDRDLEFLKQLESSDLLDLFEVLVFGKDGEKRHNEKLTSSIEYKRHGDDYAKYAERIAEELQYYGSNSFASFIKGEGVLYKEILCDVCDKLKVNYNKKTETTLIEQNMLSKILERSLEEMDDEEVKEMCDELSIKNTDNLNRQALSAATLTLFKMGGFKSYQLAVIVANAVAKTILGRGLSLAGNQVLTRTLSFLTGPVGWIITGVWTAIDIAGPAYRVTIPACIVVATLRLKTQQANGDKKSLQIESI

SEQ ID NO: 5 - protein, 54 aa, Helicobacter sp.
TEIDGDAYHKPSFNDLGLKESVLKSVYEAGFTSPSPIQEKAIPAVLQGRDVIAQ

SEQ ID NO: 6 - protein, 31 aa, Helicobacter sp.
KTAAFALPIINNLKNNHTIEALVITPTRELA

SEQ ID NO: 7 - protein, 26 aa, Helicobacter sp.
AMQISDEIFKLGKHTRTKTVCVYGGQ

SEQ ID NO: 8 - protein, 41 aa, Helicobacter sp.
VMIATPGRLLDHLKNERIHKFVPKVVVLDESDEMLDMGFLD

SEQ ID NO: 9 - protein, 31 aa, Helicobacter sp.
IFDYLPSEAQILLFSATMPEPIKRLADKILE

SEQ ID NO: 10 - protein, 23 aa, Helicobacter sp.
NEHERAEAIMRLLDTQAPKKSIV

SEQ ID NO: 11 - protein, 36 aa, Helicobacter sp.
ADELHQFLASKNYKSTALHGDMDQRDRRSSIMAFKK

SEQ ID NO: 12 - protein, 41 aa, Helicobacter sp.
GLDISGVSHVFNYHLPLNTESYIHRIGRTGRAGKKGMAITL

SEQ ID NO: 13 - protein, 31 aa, Helicobacter sp.
RAGKKGMAITLVTPLEYKELLRMQKEIDSEI

SEQ ID NO: 14 - protein, 36 aa, Helicobacter sp.
IPTINENQIIKTLHDAKVSEGIISLYEQLTEIFEPS

SEQ ID NO: 15 - protein, 21 aa, Helicobacter sp.
SQLVLKLLSLQFETSKIGLNQ

SEQ ID NO: 16 - protein, 30 aa, Helicobacter sp.
MAYKYDRDLEFLKQLESSDLLDLFEVLVFG

SEQ ID NO: 17 - protein, 38 aa, Helicobacter sp.
DYAKYAERIAEELQYYGSNSFASFIKGEGVLYKEILCD

SEQ ID NO: 18 - protein, 30 aa, Helicobacter sp.
LEEMDDEEVKEMCDELSIKNTDNLNRQALS

SEQ ID NO: 19 - protein, 41 aa, Helicobacter sp.
NRQALSAATLTLFKMGGFKSYQLAVIVANAVAKTILGRGLS

SEQ ID NO: 20 - protein, 49 aa, Helicobacter sp.
VGWIITGVWTAIDIAGPAYRVTIPACIVVATLRLKTQQANGDKKSLQIE

SEQ ID NO: 21 - DNA, 162 bp, Helicobacter sp.
acagaaattgatggtgacgcttatcataagccgagttttaatgatttgggcttaaaagaatcggttttaaaatccgtttatgaagccggcttcacttccccaagccccattcaagaaaaggccattccggctgttttgcaaggccgagatgtcatcgcacaa

SEQ ID NO: 22 - DNA, 93 bp, Helicobacter sp.
aaaaccgccgctttcgctctgcccattatcaacaaccttaaaaacaaccacaccatagaagccctagtgatcacgcccaccagagaattagcc

SEQ ID NO: 23 - DNA, 78 bp, Helicobacter sp.
gccatgcaaattagcgatgagattttcaaattgggcaaacacaccaggactaaaaccgtgtgcgtgtatggaggccag

SEQ ID NO: 24 - DNA, 123 bp, Helicobacter sp.
gtgatgatcgctacaccaggaaggctgctcgatcacttaaaaaacgaacgcatccataaatttgtgcctaaagtggtcgttttagatgaaagcgatgaaatgctggatatggggtttttagac

SEQ ID NO: 25 - DNA, 93 bp, Helicobacter sp.
atttttgactacctccctagcgaagcgcagattttgcttttttcagccacgatgccagagccgattaaaagactagcggataagattttagaa

SEQ ID NO: 26 - DNA, 69 bp, Helicobacter sp.
aatgagcatgagagggccgaagcgatcatgcgccttttagacacccaagcacccaaaaagagcattgtt

SEQ ID NO: 27 - DNA, 108 bp, Helicobacter sp.
gccgatgaattgcaccaattccttgcttctaaaaattacaaaagcaccgccttgcatggggatatggatcaaagggatcggcgctcttctatcatggcgtttaaaaaa

SEQ ID NO: 28 - DNA, 123 bp, Helicobacter sp.
gggctagatattagcggtgtaagccatgtgtttaattaccacttgcccctaaacactgagagctatatccatcgcatcgggagaaccgggcgagcgggcaaaaaaggcatggcgatcacttta

SEQ ID NO: 29 - DNA, 93 bp, Helicobacter sp.
cgagcgggcaaaaaaggcatggcgatcactttagtaacccctttagaatacaaagagcttttacgcatgcaaaaagaaattgattcagagatt

SEQ ID NO: 30 - DNA, 108 bp, Helicobacter sp.
atccccaccattaacgaaaatcagatcatcaaaaccttgcatgacgctaaagtgtctgaagggatcatcagcctttatgaacagcttaccgaaatttttgagccgtct

SEQ ID NO: 31 - DNA, 63 bp, Helicobacter sp.
tctcaattggttttaaaacttttgagtttgcagtttgaaaccagcaaaattggcttaaaccag

SEQ ID NO: 32 - DNA, 90 bp, Helicobacter sp.
atggcatacaaatatgatagagacttggaatttttaaagcaattggaatctagtgatttattggatttgtttgaggtgcttgtttttggt

SEQ ID NO: 33 - DNA, 114 bp, Helicobacter sp.
gattacgctaaatacgcagaaagaatcgctgaagagttgcaatactatgggagcaatagttttgcgagtttcattaaaggcgaaggagtcttatacaaagagattttatgcgat

SEQ ID NO: 34 - DNA, 90 bp, Helicobacter sp.
ttggaagaaatggatgatgaagaagtgaaagaaatgtgcgatgaattatccataaaaaacacggacaatttaaacagacaagccttaagc

SEQ ID NO: 35 - DNA, 108 bp, Helicobacter sp.
aacagacaagccttaagcgcggcgactttaacgctgtttaaaatggggggttttaaatcttatcaattagctgtcattgttgcgaatgcggtcgcaaaaaccattcta

SEQ ID NO: 36 - DNA, 153 bp, Helicobacter sp.
ggtcctgttggctggatcattacaggcgtatggacagcgattgatattgcagggccggcttatagggtaaccataccggcatgcattgtggttgccactttacgcctaaaaacacagcaagccaatggagataagaagtcgttgcaaatagaa

SEQ ID NO: 37 - DNA, 33 bp, Helicobacter sp.
cagaggggatccatggaattgaatcaaccacca

SEQ ID NO: 38 - DNA, 36 bp, Helicobacter sp.
cagagggtcgacttaacggcgtttgggttttttaga

SEQ ID NO: 39 - DNA, 30 bp, Helicobacter sp.
gcgggatccatggcatacaaatatgataga

SEQ ID NO: 40 - DNA, 32 bp, Helicobacter sp.
gcggtcgacttaaatggattctatttgcaacg

SEQ ID NO: 41 - DNA, 1512 bp, Helicobacter sp.
atgagaggatcgcatcaccatcaccatcacggatccatggaattgaatcaaccaccactccctacagaaattgatggtgacgcttatcataagccgagttttaatgatttgggcttaaaagaatcggttttaaaatccgtttatgaagccggcttcacttccccaagccccattcaagaaaaggccattccggctgttttgcaaggccgagatgtcatcgcacaagcccaaacaggcacaggaaaaaccgccgctttcgctctgcccattatcaacaaccttaaaaacaaccacaccatagaagccctagtgatcacgcccaccagagaattagccatgcaaattagcgatgagattttcaaattgggcaaacacaccaggactaaaaccgtgtgcgtgtatggaggccagagcgttaaaaagcaatgcgaattcattaagaaaaatccccaagtgatgatcgctacaccaggaaggctgctcgatcacttaaaaaacgaacgcatccataaatttgtgcctaaagtggtcgttttagatgaaagcgatgaaatgctggatatggggtttttagacgatattgaagagatttttgactacctccctagcgaagcgcagattttgcttttttcagccacgatgccagagccgattaaaagactagcggataagattttagaaaaccctattaaaatccatatcgctccttctaatatcactaacaccgacatcacccaacgcttttatgtgatcaatgagcatgagagggccgaagcgatcatgcgccttttagacacccaagcacccaaaaagagcattgttttcacgcgcactaaaaaagaagccgatgaattgcaccaattccttgcttctaaaaattacaaaagcaccgccttgcatggggatatggatcaaagggatcggcgctcttctatcatggcgtttaaaaaaaatgacgctgatgtgttggtggctacagatgtggcgagtcgtgggctagatattagcggtgtaagccatgtgtttaattaccacttgcccctaaacactgagagctatatccatcgcatcgggagaaccgggcgagcgggcaaaaaaggcatggcgatcactttagtaacccctttagaatacaaagagcttttacgcatgcaaaaagaaattgattcagagattgaactttttgaaatccccaccattaacgaaaatcagatcatcaaaaccttgcatgacgctaaagtgtctgaagggatcatcagcctttatgaacagcttaccgaaatttttgagccgtctcaattggttttaaaacttttgagtttgcagtttgaaaccagcaaaattggcttaaaccagcaagaaattgacgcgattcaaaaccctaaagaaaaaacgccaaaaccctctaacaaaaaaacgccccaacatgagcgagcgcgttctttcaaaaagggtcagcacagagacagacaccctaaaacaaaccattattctaaaaaacccaaacgccgt

SEQ ID NO: 42 - protein, 504 aa, Helicobacter sp.
MRGSHHHHHHGSMELNQPPLPTEIDGDAYHKPSFNDLGLKESVLKSVYEAGFTSPSPIQEKAIPAVLQGRDVIAQAQTGTGKTAAFALPIINNLKNNHTIEALVITPTRELAMQISDEIFKLGKHTRTKTVCVYGGQSVKKQCEFIKKNPQVMIATPGRLLDHLKNERIHKFVPKVVVLDESDEMLDMGFLDDIEEIFDYLPSEAQILLFSATMPEPIKRLADKILENPIKIHIAPSNITNTDITQRFYVINEHERAEAIMRLLDTQAPKKSIVFTRTKKEADELHQFLASKNYKSTALHGDMDQRDRRSSIMAFKKNDADVLVATDVASRGLDISGVSHVFNYHLPLNTESYIHRIGRTGRAGKKGMAITLVTPLEYKELLRMQKEIDSEIELFEIPTINENQIIKTLHDAKVSEGIISLYEQLTEIFEPSQLVLKLLSLQFETSKIGLNQQEIDAIQNPKEKTPKPSNKKTPQHERARSFKKGQHRDRHPKTNHYSKKPKRR

SEQ ID NO: 43 - DNA, 795 bp, Helicobacter sp.
atgagaggatcgcatcaccatcaccatcacggattcatggcatacaaatatgatagagacttggaatttttaaagcaattggaatctagtgatttattggatttgtttgaggtgcttgtttttggtaaagacggcgaaaaaagacacaatgaaaaactgaccagctccatagaatacaaaaggcatggcgatgattacgctaaatacgcagaaagaatcgctgaagagttgcaatactatgggagcaatagttttgcgagtttcattaaaggcgaaggagtcttatacaaagagattttatgcgatgtgtgcgataaattaaaggtcaattacaacaagaaaactgaaacgactttaattgaacaaaacatgctttctaaaatcttagaaagaagtttggaagaaatggatgatgaagaagtgaaagaaatgtgcgatgaattatccataaaaaacacggacaatttaaacagacaagccttaagcgcggcgactttaacgctgtttaaaatggggggttttaaatcttatcaattagctgtcattgttgcgaatgcggtcgcaaaaaccattctagggcgtggtttatcgcttgcgggcaatcaggtgcttacaagaactctgagctttttaacaggtcctgttggctggatcattacaggcgtatggacagcgattgatattgcagggccggcttatagggtaaccataccggcatgcattgtggttgccactttacgcctaaaaacacagcaagccaatggagataagaagtcgttgcaaatagaatccatt

SEQ ID NO: 44 - protein, 265 aa, Helicobacter sp.
MRGSHHHHHHGSMAYKYDRDLEFLKQLESSDLLDLFEVLVFGKDGEKRHNEKLTSSIEYKRHGDDYAKYAERIAEELQYYGSNSFASFIKGEGVLYKEILCDVCDKLKVNYNKKTETTLIEQNMLSKILERSLEEMDDEEVKEMCDELSIKNTDNLNRQALSAATLTLFKMGGFKSYQLAVIVANAVAKTILGRGLSLAGNQVLTRTLSFLTGPVGWIITGVWTAIDIAGPAYRVTIPACIVVATLRLKTQQANGDKKSLQIESI